(19) United States
(12) Patent Application Publication
(10) Pub. No.: US 2025/0303168 A1
(43) Pub. Date: Oct. 2, 2025

(54) SYSTEMS AND METHODS FOR SLEEP DISORDERED BREATHING THERAPY TITRATION

(71) Applicant: ZOLL MEDICAL CORPORATION, Chelmsford, MA (US)

(72) Inventors: Gary A. Freeman, Waltham, MA (US); Todd P. Goblish, Maple Grove, MN (US); Kristofer J. James, Eagan, MN (US); Antonios Panteleon, Nea Penteli (GR); Randy W. Westlund, St. Paul, MN (US)

(21) Appl. No.: 18/989,189

(22) Filed: Dec. 20, 2024

Related U.S. Application Data
(60) Provisional application No. 63/571,877, filed on Mar. 29, 2024.

(51) Int. Cl. A61N 1/36; A61N 1/05; A61N 1/372

(52) U.S. Cl. CPC A61N 1/3611; A61N 1/0551; A61N 1/36139; A61N 1/3614; A61N 1/37217

(57) ABSTRACT

Examples of the disclosure include a system for providing sleep-disordered-breathing treatment for a patient, the system including one or more stimulation leads configured to be implantable in the patient, receive an electrical stimulation energy, and deliver electrical stimulation to a target nerve, and at least one implantable electrical pulse generator including wireless-communication circuitry, at least one power source configured to generate the energy, stimulation circuitry configured to provide the energy to the leads, at least one sensor configured to generate a signal indicative of a degree of patient arousal, memory, and at least one processor configured to receive the signal, process signal to detect a change in the degree of patient arousal, and in response to the detected change in the degree of patient arousal, cause the stimulation circuitry to titrate the energy, including one or more adjustments between a minimum effective energy and a target maximum treatment energy.

### CROSS-REFERENCE TO RELATED APPLICATIONS

This application claims priority under 35 U.S.C. § 119(e) to U.S. Provisional Application Ser. No. 63/571,877, titled “SYSTEMS AND METHODS FOR SLEEP DISORDERED BREATHING THERAPY TITRATION,” filed on Mar. 29, 2024, which is hereby incorporated by reference in its entirety.

### BACKGROUND

At least one example in accordance with the present disclosure relates generally to breathing disorders.

Respiration in humans is subject to both voluntary and involuntary control, and several disease processes can have an impact on respiration. The autonomic nervous system regulates involuntary physiological processes, including respiration, providing sensory input, and providing motor output to the muscles, such as the diaphragm and accessory muscles. The central nervous system commands the diaphragm and other muscles in the chest and neck via the phrenic nerve to physically contract and relax, thus stimulating breathing. The central nervous system thus acts as a respiratory pacemaker by setting the breathing rate. For a sleeping person with respiration considered to be healthy and normal, the next breath may be initiated after (for example, substantially immediately after) the previous breath is exhaled. Breathing may be characterized by various parameters including, but not limited to, tidal volume, respiration rate, regularity, rhythm, and so forth.

Breathing that exhibits various characteristics that deviate from normal respiration and that may be indicative of unhealthy respiration may be described as disordered breathing. Sleep apnea is a type of sleep disordered breathing that presents as a breathing-related sleep disorder. Sleep apnea exists in several forms, including central sleep apnea (CSA), obstructive sleep apnea (OSA), and other types of breathing disorders. CSA includes apnea events characterized by an ineffective, or decreased, respiratory effort. The decreased respiratory effort or drive may include an absent respiratory effort or drive. OSA includes apnea events characterized by an obstruction of airflow. An obstruction of airflow may result from a partial or complete obstruction of the airway resulting in a reduction in airflow relative to airflow without an obstruction.

### SUMMARY

Examples of the methods and systems discussed herein are not limited in application to the details of construction and the arrangement of components set forth in the following description or illustrated in the accompanying drawings. The methods and systems may be capable of implementation in other embodiments and of being practiced or of being carried out in various ways. Examples of specific implementations are provided herein for illustrative purposes and are not intended to be limiting. Acts, components, elements, and features discussed in connection with any one or more examples may be configured to operate and/or be implemented in a similar role in any other examples.

The phraseology and terminology used herein is for the purpose of description. References to examples, embodiments, components, elements, or acts of the systems and methods herein referred to in the singular may also embrace embodiments including a plurality. Similarly, references in plural to embodiments, components, elements, or acts may be implemented as a singularity. References in the singular or plural form may therefore not be intended to limit the presently disclosed systems or methods, their components, acts, or elements. The use herein of “including,” “comprising,” “having,” “containing,” “involving,” and variations so forth, may encompass the items listed thereafter and equivalents thereof as well as additional items.

References to “or” may be construed as inclusive so that any terms described using “or” may indicate any of a single, more than one, and all of the described terms. For example, the phrase “at least one of A or B” may refer A and/or B—that is, A only, B only, or A and B together. In addition, in the event of inconsistent usages of terms between this document and documents incorporated herein by reference, the term usage in the incorporated documents is supplementary to this document. For irreconcilable differences, the term usage in this document controls.

According to at least one aspect of the present disclosure includes a system for providing sleep disordered breathing treatment for a patient, the system including: one or more stimulation leads, each lead being configured to be implantable in the patient, receive an electrical stimulation energy, and deliver electrical stimulation to a target nerve based on the received electrical stimulation energy; and at least one implantable electrical pulse generator configured to be coupled to the one or more stimulation leads and including: wireless communication circuitry, at least one power source configured to generate the electrical stimulation energy, stimulation circuitry coupled to the at least one power source and configured to provide the electrical stimulation energy to the one or more stimulation leads, at least one sensor configured to generate at least one signal indicative of a degree of arousal of the patient, memory, and at least one processor coupled to the wireless communication circuitry, the at least one power source, the stimulation circuitry, the at least one sensor, and the memory, the at least one processor being configured to: receive the at least one signal indicative of the degree of arousal of the patient, process the at least one signal to detect a change in the degree of arousal, and in response to the detected change in the degree of arousal, cause the stimulation circuitry to titrate the electrical stimulation energy, wherein the titration includes one or more adjustments of the electrical stimulation energy between a minimum effective energy and a target maximum treatment energy.

In at least one example, the at least one sensor includes at least one accelerometer. In at least one example, the at least one sensor includes at least one of a transthoracic impedance sensor, an acoustic sensor, an airflow sensor, a heart rate sensor, a blood oxygenation sensor, a muscular electrical activity sensor, or a peripheral arterial tone sensor. In at least one example, the at least one signal indicative of the change in the degree of arousal includes an acceleration signal indicative of patient movement information. In at least one example, the patient movement information includes rolling information. In at least one example, the rolling information includes a number of rolls, a frequency of rolls, a magnitude of rolls, cumulative number of rolls in a respective time period, or combinations thereof.

In at least one example, a first degree of arousal corresponds to the number of rolls, the frequency of rolls, the magnitude of rolls, the cumulative number of rolls in a first respective time period, or combinations thereof subceeding a respective threshold, and a second degree of arousal corresponds to the number of rolls, the frequency of rolls, the magnitude of rolls, the cumulative number of rolls in a second respective time period, or combinations thereof exceeding the respective threshold. In at least one example, the magnitude of rolls is indicative of movement of the patient relative to positional quadrants. In at least one example, the positional quadrants include a prone position, a supine position, a right-side position, and a left-side position. In at least one example, a first degree of arousal corresponds to patient motion within a single positional quadrant; a second degree of arousal corresponds to patient motion between the positional quadrants; and a third degree of arousal corresponds to patient motion above a threshold amount irrespective of positional quadrant.

In at least one example, the at least one processor is configured to detect a deviation of the rolling information from a baseline movement profile for the patient and titrate the electrical stimulation energy based on the deviation of the rolling information. In at least one example, the detection of the deviation of the rolling information triggers the titration of the electrical stimulation energy. In at least one example, the baseline movement profile is determined during a calibration phase before the electrical stimulation energy is provided to the patient. In at least one example, the deviation from the baseline movement profile includes one or more of: a) a present number of rolls over a present rolling window of time exceeding or subceeding a baseline number of rolls indicated by the baseline movement profile over a baseline rolling window of time by at least a threshold number of rolls, wherein the present rolling window of time is equal to the baseline rolling window of time; b) a present frequency of rolls exceeding or subceeding a baseline frequency of rolls indicated by the baseline movement profile by at least a threshold frequency of rolls; c) a present magnitude of one or more rolls exceeding or subceeding a baseline magnitude of one or more rolls indicated by the baseline movement profile by at least a threshold magnitude, wherein the present magnitude of the one or more rolls and the baseline magnitude of one or more rolls are each measured by a number of angular degrees rolled in a respective roll; or d) a present cumulative number of rolls over a present reference period of time exceeding or subceeding a baseline cumulative number of rolls indicated by the baseline movement profile over a baseline reference period of time by at least a threshold cumulative number of rolls.

In at least one example, the present reference period of time and the baseline reference period of time each include a sleeping time period between the patient's falling asleep and waking up. In at least one example, the present reference period of time and the baseline reference period of time each include a week. In at least one example, the at least one processor is configured to adjust over time one or more of the threshold number of rolls, the threshold frequency of rolls, the threshold magnitude, or the threshold cumulative number of rolls. In at least one example, the patient movement information includes translation information. In at least one example, the at least one processor is configured to provide the electrical stimulation energy as a plurality of pulse train envelopes, each pulse train envelope including a pulse train including a plurality of individual pulses, and wherein the one or more adjustments of the electrical stimulation energy include one of more adjustments of at least one of an individual pulse amplitude, a number of individual pulses in each pulse train envelope, an individual pulse width, a maximum individual pulse amplitude, an individual pulse frequency, a stimulation current, a stimulation voltage, a stimulation polarity, or stimulation energy ramps.

In at least one example, the one or more adjustments include an increase or decrease in the stimulation current by a number of milliamps or by a percentage of a present stimulation current. In at least one example, the one or more adjustments include an increase or decrease in the stimulation current by tenths of milliamps. In at least one example, the one or more adjustments include an increase or decrease in the stimulation current by hundredths of milliamps. In at least one example, an amount of adjustment for the one or more adjustments depends on a type of electrode. In at least one example, the type of electrode is a transvenous lead electrode or a nerve cuff electrode. In at least one example, the one or more adjustments include a decrease in the electrical stimulation energy from a first energy to a second energy in response to an increase in the degree of arousal, the second energy being non-zero and being equal to or greater than the minimum effective energy.

In at least one example, the one or more adjustments include an increase in the electrical stimulation energy from a first energy to a second energy over a plurality of steps, the first energy being equal to or greater than the minimum effective energy and the second energy being equal to or less than the target maximum treatment energy, wherein each step of the plurality of steps includes a change in energy. In at least one example, the one or more adjustments include a change in the electrical stimulation energy from a first energy to a second energy over a plurality of steps, and wherein the one or more adjustments include at least one of (a) an adjustment of a quantity of steps included in the plurality of steps or (b) a magnitude of at least one step of the plurality of steps.

In at least one example, the one or more adjustments include (a) an incremental decrease in a magnitude of at least one step of the plurality of steps as the electrical stimulation energy approaches the target maximum treatment energy, and (b) an incremental increase in a quantity of steps included in the plurality of steps as the electrical stimulation energy approaches the target maximum treatment energy. In at least one example, the at least one processor is further configured to: process the at least one signal to detect an absence of change in the degree of arousal, and in response to the detected absence of change in the degree of arousal, cause the stimulation circuitry to increase the electrical stimulation energy between the minimum effective energy and the target maximum treatment energy.

In at least one example, the titration of the electrical stimulation energy includes an adjustment of at least one hold time duration at a particular electrical stimulation energy. In at least one example, the at least one processor is configured to cause the stimulation circuitry to titrate the electrical stimulation energy based at least in part on the target nerve. In at least one example, the minimum effective energy includes a plurality of minimum effective energies. In at least one example, each minimum effective energy of the plurality of minimum effective energies is associated with a respective one of a prone position of the patient, a supine position of the patient, a right-side position of the patient, and a left-side position of the patient.

In at least one example, the target nerve includes an upper-airway nerve. In at least one example, the upper-airway nerve includes a hypoglossal nerve. In at least one example, the minimum effective energy includes a minimum energy that, when applied to the upper-airway nerve, increases airway patency for the patient. In at least one example, the target nerve includes at least one phrenic nerve of the patient. In at least one example, the minimum effective energy includes a minimum energy that, when applied to the at least one phrenic nerve, modulates the diaphragm of the patient. In at least one example, the at least one sensor includes at least one sensor configured to generate at least one feedback signal indicative of respiration of the patient.

In at least one example, the at least one sensor includes at least one of a transthoracic impedance sensor, a motion sensor, a pressure sensor, an acoustic sensor, an airflow sensor, a heart rate sensor, a blood oxygenation sensor, a muscular electrical activity sensor, or a peripheral arterial tone sensor. In at least one example, the at least one processor is coupled to the at least one sensor, and wherein the titration includes at least one adjustment of the electrical stimulation energy based on the at least one feedback signal. In at least one example, the at least one processor is further configured to determine an entrainment index based on the at least one feedback signal. In at least one example, the at least one processor is further configured to compare the entrainment index to a threshold value.

In at least one example, the one or more adjustments include a decrease in the electrical stimulation energy responsive to the entrainment index exceeding the threshold value. In at least one example, the one or more adjustments include an increase in the electrical stimulation energy responsive to the entrainment index subceeding the threshold value. In at least one example, the titration includes maintaining a magnitude of the electrical stimulation energy responsive to the entrainment index being within a range of values around the threshold value. In at least one example, the target maximum treatment energy is an energy at which the entrainment index is within the range of values around the threshold value.

In at least one example, the system includes at least two stimulation leads, wherein the target nerve for at least one first stimulation lead is an upper-airway nerve and the target nerve for at least one second stimulation lead is a phrenic nerve. In at least one example, the at least one implantable electrical pulse generator is configured to support dual channel operations, wherein a first channel is configured to control stimulation of at least one upper-airway nerve and a second channel is configured to control stimulation of at least one phrenic nerve. In at least one example, the titration of the electrical stimulation energy includes an adjustment of at least one of the minimum effective energy or the target maximum treatment energy over time.

In at least one example, the at least one processor is configured to cause the stimulation circuitry to titrate the electrical stimulation energy based on a predicted patient response to the change in the degree of arousal. In at least one example, the at least one processor is configured to detect a change in patient position and cause the stimulation circuitry to titrate the electrical stimulation energy based on a predicted patient response to the change in patient position. In at least one example, the at least one implantable electrical pulse generator includes a transceiver configured to communicate according to a Bluetooth® protocol. In at least one example, the at least one sensor includes at least one transthoracic impedance sensor.

Examples of the disclosure include a method of providing sleep disordered breathing treatment for a patient, the method including: receiving, by at least one lead, an electrical stimulation energy; delivering, by the at least one lead based on the electrical stimulation energy, electrical stimulation to a target nerve of the patient; receiving at least one signal indicative of a change in a degree of arousal of the patient from at least one sensor; processing the least one signal to detect a change in the degree of arousal; and titrating the electrical stimulation energy based on the change in the degree of arousal, wherein titrating the electrical stimulation energy includes one or more adjustments of the electrical stimulation energy between a minimum effective energy and a target maximum treatment energy.

In at least one example, the at least one sensor includes at least one accelerometer. In at least one example, the at least one sensor includes at least one of a transthoracic impedance sensor, an acoustic sensor, an airflow sensor, a heart rate sensor, a blood oxygenation sensor, a muscular electrical activity sensor, or a peripheral arterial tone sensor. In at least one example, the at least one signal indicative of the change in the degree of arousal includes an acceleration signal indicative of patient movement information. In at least one example, the patient movement information includes rolling information.

In at least one example, the rolling information includes a number of rolls, a frequency of rolls, a magnitude of rolls, a cumulative number of rolls in a respective time period, or combinations thereof. In at least one example, a first degree of arousal corresponds to the number of rolls, the frequency of rolls, the magnitude of rolls, the cumulative number of rolls in a first respective time period, or combinations thereof subceeding a respective threshold, and a second degree of arousal corresponds to the number of rolls, the frequency of rolls, the magnitude of rolls, the cumulative number of rolls in a second respective time period, or combinations thereof exceeding the respective threshold.

In at least one example, the magnitude of rolls is indicative of movement of the patient relative to positional quadrants. In at least one example, the positional quadrants include a prone position, a supine position, a right-side position, and a left-side position. In at least one example, a first degree of arousal corresponds to patient motion within a first positional quadrant; a second degree of arousal corresponds to patient motion between the positional quadrants; and a third degree of arousal corresponds to patient motion above a threshold amount irrespective of positional quadrant. In at least one example, the method includes detecting a deviation of the rolling information from a baseline movement profile for the patient and titrating the electrical stimulation energy based on the deviation of the rolling information.

In at least one example, the detection of the deviation of the rolling information triggers the titration of the electrical stimulation energy. In at least one example, the baseline movement profile is determined during a calibration phase before the electrical stimulation energy is provided to the patient. In at least one example, the deviation from the baseline movement profile includes one or more of: a) a present number of rolls over a present rolling window of time exceeding or subceeding a baseline number of rolls indicated by the baseline movement profile over a baseline rolling window of time by at least a threshold number of rolls, wherein the present rolling window of time is equal to the baseline rolling window of time; b) a present frequency of rolls exceeding or subceeding a baseline frequency of rolls indicated by the baseline movement profile by at least a threshold frequency of rolls; c) a present magnitude of one or more rolls exceeding or subceeding a baseline magnitude of one or more rolls indicated by the baseline movement profile by at least a threshold magnitude, wherein the present magnitude of the one or more rolls and the baseline magnitude of one or more rolls are each measured by a number of angular degrees rolled in a respective roll; or d) a present cumulative number of rolls over a present reference period of time exceeding or subceeding a baseline cumulative number of rolls indicated by the baseline movement profile over a baseline reference period of time by at least a threshold cumulative number of rolls.

In at least one example, the present reference period of time and the baseline reference period of time each include a sleeping time period between the patient's falling asleep and waking up. In at least one example, the present reference period of time and the baseline reference period of time each include a week. In at least one example, the method includes adjusting over time one or more of the threshold number of rolls, the threshold frequency of rolls, the threshold magnitude, or the threshold cumulative number of rolls. In at least one example, the patient movement information includes translation information.

In at least one example, the method includes providing the electrical stimulation energy as a plurality of pulse train envelopes, each pulse train envelope including a pulse train including a plurality of individual pulses, and wherein the one or more adjustments of the electrical stimulation energy include one of more adjustments of at least one of an individual pulse amplitude, a number of individual pulses in each pulse train envelope, an individual pulse width, a maximum individual pulse amplitude, an individual pulse frequency, a stimulation current, a stimulation voltage, a stimulation polarity, or stimulation energy ramps. In at least one example, the one or more adjustments include an increase or decrease in the stimulation current by a number of milliamps or by a percentage of a present stimulation current.

In at least one example, the one or more adjustments include an increase or decrease in the stimulation current by tenths of milliamps. In at least one example, the one or more adjustments include an increase or decrease in the stimulation current by hundredths of milliamps. In at least one example, an amount of adjustment for the one or more adjustments depends on a type of electrode. In at least one example, the type of electrode is a transvenous lead electrode or a nerve cuff electrode. In at least one example, the one or more adjustments include a decrease in the electrical stimulation energy from a first energy to a second energy in response to an increase in the degree of arousal, the second energy being non-zero and being equal to or greater than the minimum effective energy.

In at least one example, the one or more adjustments include an increase in the electrical stimulation energy from a first energy to a second energy over a plurality of steps, the first energy being equal to or greater than the minimum effective energy and the second energy being equal to or less than the target maximum treatment energy, wherein each step of the plurality of steps includes a change in energy. In at least one example, the one or more adjustments include at least one of (a) an adjustment of a quantity of steps included in the plurality of steps or (b) a magnitude of at least one step of the plurality of steps. In at least one example, the one or more adjustments include (a) an incremental decrease in a magnitude of at least one step of the plurality of steps as the electrical stimulation energy approaches the target maximum treatment energy, and (b) an incremental increase in a quantity of steps included in the plurality of steps as the electrical stimulation energy approaches the target maximum treatment energy.

In at least one example, the method includes processing the at least one signal to detect an absence of change in the degree of arousal, and in response to the detected absence of change in the degree of arousal, increasing the electrical stimulation energy between the minimum effective energy and the target maximum treatment energy. In at least one example, the titration of the electrical stimulation energy includes an adjustment of at least one hold time duration at a particular electrical stimulation energy. In at least one example, the method includes titrating the electrical stimulation energy based at least in part on the target nerve. In at least one example, the minimum effective energy includes a plurality of minimum effective energies.

In at least one example, each minimum effective energy of the plurality of minimum effective energies is associated with a respective one of a prone position of the patient, a supine position of the patient, a right-side position of the patient, and a left-side position of the patient.

In at least one example, the target nerve includes an upper-airway nerve. In at least one example, the upper-airway nerve includes a hypoglossal nerve. In at least one example, the minimum effective energy includes a minimum energy that, when applied to the upper-airway nerve, increases airway patency for the patient. In at least one example, the target nerve includes at least one phrenic nerve of the patient.

In at least one example, the minimum effective energy includes a minimum energy that, when applied to the at least one phrenic nerve, modulates the diaphragm of the patient. In at least one example, the method includes generating, by at least one sensor, at least one feedback signal indicative of respiration of the patient. In at least one example, the at least one sensor includes at least one of a transthoracic impedance sensor, a motion sensor, a pressure sensor, an acoustic sensor, an airflow sensor, a heart rate sensor, a blood oxygenation sensor, a muscular electrical activity sensor, or a peripheral arterial tone sensor. In at least one example, the titration includes at least one adjustment of the electrical stimulation energy based on the at least one feedback signal.

In at least one example, the method includes determining an entrainment index based on the at least one feedback signal. In at least one example, the method includes comparing the entrainment index to a threshold value. In at least one example, the one or more adjustments include a decrease in the electrical stimulation energy responsive to the entrainment index exceeding the threshold value. In at least one example, the one or more adjustments include an increase in the electrical stimulation energy responsive to the entrainment index subceeding the threshold value. In at least one example, the titration includes maintaining a magnitude of the electrical stimulation energy responsive to the entrainment index being within a range of values around the threshold value.

In at least one example, the target maximum treatment energy is an energy at which the entrainment index is within the range of values around the threshold value. In at least one example, the titration of the electrical stimulation energy includes an adjustment of at least one of the minimum effective energy or the target maximum treatment energy over time. In at least one example, the method includes supporting dual channel operations, wherein a first channel is configured to control stimulation of at least one upper-airway nerve and a second channel is configured to control stimulation of at least one phrenic nerve. In at least one example, the method includes titrating the electrical stimulation energy based on a predicted patient response to the change in the degree of arousal. In at least one example, the method includes detecting a change in patient position and titrating the electrical stimulation energy based on a predicted patient response to the change in patient position. In at least one example, the method includes exchanging communications according to a Bluetooth® protocol.

Examples of the disclosure include a system for providing sleep disordered breathing treatment for a patient, the system including: one or more stimulation leads, each lead being configured to: be implantable in the patient, receive an electrical stimulation energy, and deliver electrical stimulation to a target nerve based on the received electrical stimulation energy; and at least one implantable electrical pulse generator configured to be coupled to the one or more stimulation leads and including: wireless communication circuitry, at least one power source configured to generate the electrical stimulation energy, stimulation circuitry coupled to the at least one power source and configured to provide the electrical stimulation energy to the one or more stimulation leads, at least one sensor configured to generate at least one signal indicative of at least one sleep parameter of the patient, memory, and at least one processor coupled to the wireless communication circuitry, the at least one power source, the stimulation circuitry, the at least one sensor, and the memory, the at least one processor being configured to: receive the at least one signal indicative of the at least one sleep parameter, process the at least one signal to detect a change in the at least one sleep parameter, and in response to the detected change in the at least one sleep parameter, cause the stimulation circuitry to adjust an electrical parameter of the electrical stimulation energy.

In at least one example, the at least one sleep parameter of the patient includes a degree of arousal. In at least one example, the at least one signal is indicative of a change in the degree of arousal and includes an acceleration signal indicative of patient movement information. In at least one example, the patient movement information includes rolling information. In at least one example, the rolling information includes a number of rolls, a frequency of rolls, a magnitude of rolls, a cumulative number of rolls in a respective time period, or combinations thereof. In at least one example, a first degree of arousal corresponds to the number of rolls, the frequency of rolls, the magnitude of rolls, the cumulative number of rolls in a first respective time period, or combinations thereof subceeding a respective threshold, and a second degree of arousal corresponds to the number of rolls, the frequency of rolls, the magnitude of rolls, the cumulative number of rolls in a second respective time period, or combinations thereof exceeding the respective threshold.

In at least one example, the magnitude of rolls is indicative of movement of the patient relative to positional quadrants. In at least one example, the positional quadrants include a prone position, a supine position, a right-side position, and a left-side position. In at least one example, a first degree of arousal corresponds to patient motion within a first positional quadrant; a second degree of arousal corresponds to patient motion between the positional quadrants; and a third degree of arousal corresponds to patient motion above a threshold amount irrespective of positional quadrant. In at least one example, the at least one processor is configured to provide the electrical stimulation energy as a plurality of pulse train envelopes, each pulse train envelope including a pulse train including a plurality of individual pulses, and wherein adjusting the electrical parameter of the electrical stimulation energy includes adjusting at least one of an individual pulse amplitude, a number of individual pulses in each pulse train envelope, an individual pulse width, a maximum individual pulse amplitude, an individual pulse frequency, a stimulation current, a stimulation voltage, a stimulation polarity, or stimulation energy ramps.

In at least one example, the adjusting includes an increase or decrease in the stimulation current by a number of milliamps or by a percentage of a present stimulation current. In at least one example, the adjusting includes an increase or decrease in the stimulation current by tenths of milliamps. In at least one example, the adjusting includes an increase or decrease in the stimulation current by hundredths of milliamps. In at least one example, an amount of adjustment for the adjusting depends on a type of electrode. In at least one example, the type of electrode is a transvenous lead electrode or a nerve cuff electrode. In at least one example, the adjustment includes a decrease in the electrical stimulation energy from a first energy to a second energy in response to an increase in the degree of arousal, the second energy being non-zero and being equal to or greater than a minimum effective energy.

In at least one example, the at least one processor is further configured to: process the at least one signal to detect an absence of change in the degree of arousal, and in response to the detected absence of change in the degree of arousal, cause the stimulation circuitry to increase the electrical stimulation energy between a minimum effective energy and a target maximum treatment energy. In at least one example, the adjustment of the electrical parameter of the electrical stimulation energy includes one or more adjustments of one or more of a stimulation voltage, a stimulation pulse duration, an electrical stimulation energy duty cycle, an electrical stimulation energy frequency content, or a stimulation pulse leading ramp slope.

In at least one example, the adjustment of the electrical parameter of the electrical stimulation energy includes one or more adjustments of the electrical stimulation energy between a minimum effective energy and a target maximum treatment energy. In at least one example, the one or more adjustments include a decrease in the electrical stimulation energy from a first energy to a second energy in response to an increase in a degree of arousal, the second energy being non-zero and being equal to or greater than the minimum effective energy. In at least one example, the one or more adjustments include a decrease in the electrical stimulation energy from a first energy to a second energy in response to an increase in a degree of arousal, the second energy being non-zero and being equal to or greater than the minimum effective energy.

In at least one example, the one or more adjustments include an increase in the electrical stimulation energy from a first energy to a second energy over a plurality of steps, the first energy being equal to or greater than the minimum effective energy and the second energy being equal to or less than the target maximum treatment energy, wherein each step of the plurality of steps includes a change in energy. In at least one example, the one or more adjustments include a change in the electrical stimulation energy from a first energy to a second energy over a plurality of steps, and wherein the one or more adjustments include at least one of (a) an adjustment of a quantity of steps included in the plurality of steps or (b) a magnitude of at least one step of the plurality of steps. In at least one example, the one or more adjustments include at least one of (a) an incremental decrease in a magnitude of at least one step of the plurality of steps as the electrical stimulation energy approaches the target maximum treatment energy, or (b) an incremental increase in a quantity of steps included in the plurality of steps as the electrical stimulation energy approaches the target maximum treatment energy.

In at least one example, the at least one processor is configured to cause the stimulation circuitry to titrate the electrical stimulation energy based at least in part on the target nerve. In at least one example, the minimum effective energy includes a plurality of minimum effective energies. In at least one example, each minimum effective energy of the plurality of minimum effective energies is associated with a respective one of a prone position of the patient, a supine position of the patient, a right-side position of the patient, and a left-side position of the patient.

In at least one example, the target nerve includes an upper-airway nerve. In at least one example, the upper-airway nerve includes a hypoglossal nerve. In at least one example, the minimum effective energy includes a minimum energy that, when applied to the upper-airway nerve, increases airway patency for the patient.

In at least one example, the target nerve includes at least one phrenic nerve of the patient. In at least one example, the minimum effective energy includes a minimum energy that, when applied to the at least one phrenic nerve, modulates the diaphragm of the patient. In at least one example, the at least one sensor includes at least one sensor configured to generate at least one feedback signal indicative of respiration of the patient. In at least one example, the at least one sensor includes at least one of a transthoracic impedance sensor, a motion sensor, a pressure sensor, an acoustic sensor, an airflow sensor, a heart rate sensor, a blood oxygenation sensor, a muscular electrical activity sensor, or a peripheral arterial tone sensor. In at least one example, the at least one processor is coupled to the at least one sensor, and wherein the titration includes at least one adjustment of the electrical stimulation energy based on the at least one feedback signal.

In at least one example, the at least one processor is further configured to determine an entrainment index based on the at least one feedback signal. In at least one example, the at least one processor is further configured to compare the entrainment index to a threshold value. In at least one example, the one or more adjustments include a decrease in the electrical stimulation energy responsive to the entrainment index exceeding the threshold value. In at least one example, the one or more adjustments include an increase in the electrical stimulation energy responsive to the entrainment index subceeding the threshold value. In at least one example, the titration includes maintaining a magnitude of the electrical stimulation energy responsive to the entrainment index being within a range of values around the threshold value. In at least one example, the target maximum treatment energy is an energy at which the entrainment index is within the range of values around the threshold value.

In at least one example, the titration includes maintaining a magnitude of the electrical stimulation energy responsive to the entrainment index being within a range of values around the threshold value. In at least one example, the target maximum treatment energy is an energy at which the entrainment index is within the range of values around the threshold value. In at least one example, the system includes at least two stimulation leads, wherein the target nerve for at least one first stimulation lead is an upper-airway nerve and the target nerve for at least one second stimulation lead is a phrenic nerve. In at least one example, the at least one implantable electrical pulse generator is configured to support dual channel operations, wherein a first channel is configured to control stimulation of at least one upper-airway nerve and a second channel is configured to control stimulation of at least one phrenic nerve. In at least one example, the adjustment of the electrical parameter includes an adjustment of at least one of a minimum effective energy or a target maximum treatment energy over time.

In at least one example, the at least one sensor includes at least one accelerometer. In at least one example, the at least one sensor includes at least one of a transthoracic impedance sensor, an acoustic sensor, an airflow sensor, a heart rate sensor, a blood oxygenation sensor, a muscular electrical activity sensor, or a peripheral arterial tone sensor. In at least one example, the at least one signal indicative of the change in the at least one sleep parameter includes an acceleration signal indicative of patient movement information. In at least one example, the patient movement information includes rolling information. In at least one example, the at least one processor is configured to detect a deviation of the rolling information from a baseline movement profile for the patient and titrate the electrical stimulation energy based on the deviation of the rolling information.

In at least one example, the detection of the deviation of the rolling information triggers the titration of the electrical stimulation energy. In at least one example, the baseline movement profile is determined during a calibration phase before the electrical stimulation energy is provided to the patient. In at least one example, the deviation from the baseline movement profile includes one or more of: a) a present number of rolls over a present rolling window of time exceeding or subceeding a baseline number of rolls indicated by the baseline movement profile over a baseline rolling window of time by at least a threshold number of rolls, wherein the present rolling window of time is equal to the baseline rolling window of time; b) a present frequency of rolls exceeding or subceeding a baseline frequency of rolls indicated by the baseline movement profile by at least a threshold frequency of rolls; c) a present magnitude of one or more rolls exceeding or subceeding a baseline magnitude of one or more rolls indicated by the baseline movement profile by at least a threshold magnitude, wherein the present magnitude of the one or more rolls and the baseline magnitude of one or more rolls are each measured by a number of angular degrees rolled in a respective roll; or d) a present cumulative number of rolls over a present reference period of time exceeding or subceeding a baseline cumulative number of rolls indicated by the baseline movement profile over a baseline reference period of time by at least a threshold cumulative number of rolls.

In at least one example, the present reference period of time and the baseline reference period of time each include a sleeping time period between the patient's falling asleep and waking up. In at least one example, the present reference period of time and the baseline reference period of time each include a week. In at least one example, the at least one processor is configured to adjust over time one or more of the threshold number of rolls, the threshold frequency of rolls, the threshold magnitude, or the threshold cumulative number of rolls. In at least one example, the patient movement information includes translation information. In at least one example, the at least one processor is further configured to: process the at least one signal to detect an absence of change in the at least one sleep parameter, and in response to the detected absence of change in the at least one sleep parameter, cause the stimulation circuitry to increase the electrical stimulation energy between a minimum effective energy and a target maximum treatment energy.

In at least one example, the adjustment of the electrical parameter includes an adjustment of at least one hold time duration at a particular electrical stimulation energy. In at least one example, the at least one processor is configured to cause the stimulation circuitry to adjust the electrical stimulation energy based at least in part on the target nerve. In at least one example, the at least one processor is configured to adjust the electrical parameter of the electrical stimulation energy based on a predicted patient response to the change in the at least one sleep parameter. In at least one example, the at least one processor is configured to detect a change in patient position and cause the stimulation circuitry to adjust the electrical parameter of the electrical stimulation energy based on a predicted patient response to the change in patient position. In at least one example, the at least one implantable electrical pulse generator includes a transceiver configured to communicate according to a Bluetooth® protocol.

Examples of the disclosure include a system for providing sleep disordered breathing treatment for a patient, the system including: one or more stimulation leads, each lead being configured to: be implantable in the patient, receive an electrical stimulation energy, and wherein at least one lead is configured to deliver electrical stimulation to at least one phrenic nerve based on the received electrical stimulation energy; and at least one implantable electrical pulse generator configured to be coupled to the one or more stimulation leads and including: wireless communication circuitry, at least one power source configured to generate the electrical stimulation energy, stimulation circuitry coupled to the at least one power source and configured to provide the electrical stimulation energy to the one or more stimulation leads, at least one sensor configured to generate at least one signal indicative of a degree of arousal of the patient, memory, and at least one processor coupled to the wireless communication circuitry, the at least one power source, the stimulation circuitry, the at least one sensor, and the memory, the at least one processor being configured to: receive the at least one signal indicative of the at least one sleep parameter, process the at least one signal to detect a change in the degree of arousal, and in response to the detected change in the degree of arousal, cause the stimulation circuitry to titrate the electrical stimulation energy, wherein the titration includes one or more adjustments of the electrical stimulation energy between a minimum effective energy and a target maximum treatment energy.

In at least one example, the at least one sensor includes at least one accelerometer. In at least one example, the at least one sensor includes at least one transthoracic impedance sensor. In at least one example, the at least one signal indicative of the change in the degree of arousal includes an acceleration signal indicative of patient movement information. In at least one example, the patient movement information includes rolling information. In at least one example, the rolling information includes a number of rolls, a frequency of rolls, a magnitude of rolls, a cumulative number of rolls in a respective time period, or combinations thereof. In at least one example, a first degree of arousal corresponds to the number of rolls, the frequency of rolls, the magnitude of rolls, the cumulative number of rolls in a first respective time period, or combinations thereof subceeding a respective threshold, and a second degree of arousal corresponds to the number of rolls, the frequency of rolls, the magnitude of rolls, the cumulative number of rolls in a second respective time period, or combinations thereof exceeding the respective threshold.

In at least one example, the magnitude of rolls is indicative of movement of the patient relative to positional quadrants. In at least one example, the positional quadrants include a prone position, a supine position, a right-side position, and a left-side position. In at least one example, a first degree of arousal corresponds to patient motion within a first positional quadrant; a second degree of arousal corresponds to patient motion between the positional quadrants; and a third degree of arousal corresponds to patient motion above a threshold amount irrespective of positional quadrant. In at least one example, the at least one processor is configured to detect a deviation of the rolling information from a baseline movement profile for the patient and titrate the electrical stimulation energy based on the deviation of the rolling information. In at least one example, the detection of the deviation of the rolling information triggers the titration of the electrical stimulation energy.

In at least one example, the baseline movement profile is determined during a calibration phase before the electrical stimulation energy is provided to the patient. In at least one example, the deviation from the baseline movement profile includes one or more of: a) a present number of rolls over a present rolling window of time exceeding or subceeding a baseline number of rolls indicated by the baseline movement profile over a baseline rolling window of time by at least a threshold number of rolls, wherein the present rolling window of time is equal to the baseline rolling window of time; b) a present frequency of rolls exceeding or subceeding a baseline frequency of rolls indicated by the baseline movement profile by at least a threshold frequency of rolls; c) a present magnitude of one or more rolls exceeding or subceeding a baseline magnitude of one or more rolls indicated by the baseline movement profile by at least a threshold magnitude, wherein the present magnitude of the one or more rolls and the baseline magnitude of one or more rolls are each measured by a number of angular degrees rolled in a respective roll; or d) a present cumulative number of rolls over a present reference period of time exceeding or subceeding a baseline cumulative number of rolls indicated by the baseline movement profile over a baseline reference period of time by at least a threshold cumulative number of rolls.

In at least one example, the present reference period of time and the baseline reference period of time each include a sleeping time period between the patient's falling asleep and waking up. In at least one example, the present reference period of time and the baseline reference period of time each include a week. In at least one example, the at least one processor is configured to adjust over time one or more of the threshold number of rolls, the threshold frequency of rolls, the threshold magnitude, or the threshold cumulative number of rolls. In at least one example, the patient movement information includes translation information. In at least one example, the one or more adjustments include a decrease in the electrical stimulation energy from a first energy to a second energy in response to an increase in the degree of arousal, the second energy being non-zero and being equal to or greater than the minimum effective energy.

In at least one example, the one or more adjustments include an increase in the electrical stimulation energy from a first energy to a second energy over a plurality of steps, the first energy being equal to or greater than the minimum effective energy and the second energy being equal to or less than the target maximum treatment energy, wherein each step of the plurality of steps includes a change in energy. In at least one example, the one or more adjustments include a change in the electrical stimulation energy from a first energy to a second energy over a plurality of steps, and wherein the one or more adjustments include at least one of (a) an adjustment of a quantity of steps included in the plurality of steps or (b) a magnitude of at least one step of the plurality of steps.

In at least one example, the one or more adjustments include (a) an incremental decrease in a magnitude of the at least one step of the plurality of steps as the electrical stimulation energy approaches the target maximum treatment energy, and (b) an incremental increase in a quantity of steps included in the plurality of steps as the electrical stimulation energy approaches the target maximum treatment energy. In at least one example, the at least one processor is further configured to: process the at least one signal to detect an absence of change in the degree of arousal, and in response to the detected absence of change in the degree of arousal, cause the stimulation circuitry to increase the electrical stimulation energy between the minimum effective energy and the target maximum treatment energy.

In at least one example, the titration of the electrical stimulation energy includes an adjustment of at least one hold time duration at a particular electrical stimulation energy. In at least one example, the minimum effective energy includes a minimum energy that, when applied to the at least one phrenic nerve, modulates the diaphragm of the patient. In at least one example, the minimum effective energy includes a plurality of minimum effective energies. In at least one example, each minimum effective energy of the plurality of minimum effective energies is associated with a respective one of a prone position of the patient, a supine position of the patient, a right-side position of the patient, and a left-side position of the patient. In at least one example, the at least one sensor includes at least one sensor configured to generate at least one feedback signal indicative of respiration of the patient.

In at least one example, the at least one sensor includes at least one transthoracic impedance sensor. In at least one example, the at least one processor is coupled to the at least one sensor, and wherein the titration includes at least one adjustment of the electrical stimulation energy based on the at least one feedback signal. In at least one example, the at least one processor is further configured to determine an entrainment index based on the at least one feedback signal. In at least one example, the at least one processor is further configured to compare the entrainment index to a threshold value. In at least one example, the one or more adjustments include a decrease in the electrical stimulation energy responsive to the entrainment index exceeding the threshold value. In at least one example, the one or more adjustments include an increase in the electrical stimulation energy responsive to the entrainment index subceeding the threshold value.

In at least one example, the titration includes maintaining a magnitude of the electrical stimulation energy responsive to the entrainment index being within a range of values around the threshold value. In at least one example, the target maximum treatment energy is an energy at which the entrainment index is within the range of values around the threshold level. In at least one example, the stimulation circuitry is configured to provide the electrical stimulation energy to the one or more stimulation leads in an asynchronous mode. In at least one example, the at least one lead is at least one first lead and wherein the system further includes at least one second lead configured to deliver electrical stimulation to an upper-airway nerve. In at least one example, the upper-airway nerve includes a hypoglossal nerve.

In at least one example, the at least one implantable electrical pulse generator is configured to support dual channel operations, wherein a first channel is configured to control stimulation via the at least one first lead and a second channel is configured to control stimulation via the at least one second lead. In at least one example, the at least one processor is configured to cause the stimulation circuitry to titrate the electrical stimulation energy for each channel of the first channel and the second channel based at least in part on a target nerve, wherein the target nerve for the first channel is the at least one phrenic nerve and the target nerve for the second channel is the upper-airway nerve. In at least one example, the titration of the electrical stimulation energy includes an adjustment of at least one of the minimum effective energy or the target maximum treatment energy over time. In at least one example, the at least one processor is configured to cause the stimulation circuitry to titrate the electrical stimulation energy based on a predicted patient response to the change in the degree of arousal.

### BRIEF DESCRIPTION OF THE DRAWINGS

Various aspects of at least one embodiment are discussed below with reference to the accompanying figures, which may not be drawn to scale. The figures are included to provide an illustration and a further understanding of the various aspects and embodiments, and are incorporated in and constitute a part of this specification, but are not intended as a definition of the limits of any particular embodiment. The drawings, together with the remainder of the specification, serve to explain principles and operations of the described and claimed aspects and embodiments. In the figures, each identical or substantially similar component that is illustrated in various figures may be represented by a like numeral. For purposes of clarity, not every component may be labeled in every figure. In the figures:

FIG. 1 illustrates a block diagram of a disordered breathing therapy system according to an example;

FIG. 2 illustrates a perspective view of a disordered breathing therapy system;

FIG. 3 illustrates implantable components of a disordered breathing therapy system according to an example;

FIG. 4 illustrates a diagram of a positional-quadrant system according to an example;

FIG. 5 illustrates a simplified graph depicting therapy titration independent of modulations in wakefulness according to an example;

FIG. 6 illustrates a simplified graph depicting therapy titration responsive to modulations in wakefulness for a sleeping patient according to another example;

FIG. 7 illustrates a simplified graph of a stimulation energy titration according to an example;

FIG. 8A illustrates a more detailed graph of a stimulation energy titration according to an example;

FIG. 8B illustrates stimulation energy titration with variations in minimum and maximum treatment currents according to an example;

FIG. 9 illustrates a more detailed graph of multiple stimulation currents according to an example;

FIG. 10A illustrates a simplified graph of a stimulation energy titrated according to predicted patient apnea responses according to an example;

FIG. 10B illustrates a simplified graph of a stimulation energy titrated according to predicted patient apnea responses according to an example;

FIG. 11A illustrates a process of calibrating, delivering, and adjusting therapy to a patient according to an example;

FIG. 11B illustrates a process of calibrating, delivering, and adjusting therapy to a patient according to another example;

FIG. 12 illustrates a process of treating disordered breathing in a patient according to an example;

FIG. 13 illustrates an entrainment index calculation according to an example;

FIG. 14 illustrates a graphical depiction of an electrical stimulation pulse train according to an example; and

FIG. 15 illustrates a block diagram of a computing device according to an example.

### DETAILED DESCRIPTION

In order to treat disordered breathing and associated sleep apneas, a disordered-breathing-treatment system may provide electrical stimulation of various nerves. Treatment of sleep disordered breathing, or disordered breathing associated with sleep apnea, generally occurs during a daily sleep cycle of a person, or patient, exhibiting the sleep disordered breathing. During this daily sleep cycle, a person may have intermittent waking periods and may experience varying degrees of wakefulness. For example, a person may go to bed at 10 PM and then wake up for the day at 7 AM but in between these times, that person may fully wake up, for example, to use the bathroom or drink water, and then resume sleeping. Additionally, that person may slightly arouse, for example, during a change in sleeping position or in response to some discomfort, without fully waking up. The arousal may be independent of the disordered breathing therapy and/or may be in response to the disordered breathing therapy. The degree of arousal, or wakefulness, may determine a person's tolerance for various levels of electrical stimulation energy. For example, the person may better tolerate a higher stimulation energy when asleep than when slightly aroused and may better tolerate a higher stimulation energy when slightly aroused than when fully aroused. In some instances, the therapy itself may provoke an increase in the degree of arousal. Such an increase in the degree of arousal may provide an indication of a reduced tolerance for a particular stimulation energy resulting in the arousal. Furthermore, an amount of stimulation energy needed in order to provide effective sleep apnea therapy may depend in part on a patient's position and/or movement during treatment.

The system described herein enables a dynamic and customized therapy titration, for example, based on sleep movement indications of patient wakefulness and/or sleep position, that maximizes the amount of time that therapy is provided during a patient's daily sleeping cycle. Such benefits may be achieved with granular adjustments in therapy parameters that can, for example, increase or decrease a stimulation energy in an energy range above a minimum effective energy or non-effective energy (just below the minimum effective energy, for example) in coordination with the patient's degree of arousal, patient movement (which may be sleep movement), and/or patient position (which may be a sleeping position). Furthermore, the system described herein may titrate therapy according to a particular form or mode of sleep apnea and the effect of sleep movement and/or sleep position on that particular form or mode of sleep apnea. For example, sleeping position may have different effects on central sleep apnea as compared with obstructive sleep apnea.

The system described herein may provide a closed-loop response to patient movement indications of patient wakefulness and/or sleep position by automatically titrating electrical stimulation therapy based on sensor measurements, such as accelerometer measurements indicative of sleep movement and/or sleeping position. Patient movement that occurs during sleep may, in turn, be indicative of a degree of arousal or wakefulness. Tailoring the therapy titration to the individual with such a closed-loop response may maximize the time that the patient is receiving effective therapy. Sleep movement data as an indicator of wakefulness may be indicative of a sleep apnea patient's ability to best tolerate a particular stimulation energy.

A higher state of arousal may correspond to a more wakeful state characterized by increased patient movement and/or activity and/or particular patient sleeping position(s). A lower state of arousal may correspond to a less wakeful state characterized by less patient movement and/or activity than the lower state of arousal and/or particular patient sleeping position(s). By reducing stimulation energy when a patient is in the higher state of arousal, for example as indicated by sleep movement data, and increasing stimulation energy when the patient is in the lower state of arousal, for example as indicated by sleep movement data, but maintaining at least a minimum effective energy (that is, not reducing the stimulation energy to zero), the system described herein may maximize a duration and quality of a patient's sleep without increasing or contributing to an increase in patient discomfort. Further, by reducing a level of stimulation energy when such a reduced energy can still produce a desired therapeutic effect, the system reduces the likelihood that the therapy itself will arouse the patient and preserves battery life for the implanted system. For example, changes in patient position may reduce a degree of obstruction or an effort to breathe, thus enabling effective therapy at a reduced energy. Additionally, titrations between close values of stimulation energy reduce the time that the patient is receiving a ramp-up energy to an effective value and thus further maximizes the benefits of therapy. The ramp-up energy may be needed for titrations between very different energy values (for example, between zero and a minimum effective energy) in order to prevent discomfort for the patient.

As an additional advantage, the system described herein may titrate therapy based on sleeping position to account for the effects of sleep position on sleep disordered breathing. For example, sleep position may determine a minimum effective energy and/or a proclivity to sleep apnea events. For instance, in obstructive sleep apnea, a minimum effective energy to effect meaningful airway modulation may be different in a supine position as compared with a prone position. Also, a patient may experience more sleep apnea events in one position as compared with another. As another example, in central sleep apnea, a position of a transvenous lead electrode may shift relative to a target nerve with changes in patient position and therapy titration based on patient position may account for these changes.

Another advantage that may be realized by the system described herein is an ability to leverage analysis of patient position and/or arousal to predict a need for therapy and/or an effective stimulation energy. In particular, when correlated with sleep apnea data, such an analysis may enable the system to proactively increase or decrease stimulation energy in expectation of a sleep apnea event or an arousal.

Additionally, the implantable pulse generator (IPG) programming may be tailored or customized to particular patient constraints. For example, if a patient is known to have a physical condition that prohibits sleeping in particular positions, an implanted device may be programmed to account for such a prohibition. For example, a patient with a hip injury may never sleep on their side and thus electrical stimulation therapy titration specific to side-sleeping would not be indicated for that patient.

As a further advantage, titration in response to patient position and/or degree of arousal, or wakefulness, may enable the patient to control therapy. For example, a patient may roll or sit-up to cause an increase or decrease in stimulation energy based on their ability to sleep and/or their ability to tolerate therapy.

Although sleep apnea corresponds to disordered breathing that occurs during sleep cycles for a patient, disordered breathing may also occur outside of sleep cycles. In other words, a person may be completely awake, and may even be in a position not typically associated with sleep, such as standing, and may still experience disordered breathing. Thus, although sleeping may increase a likelihood of disordered breathing, sleeping is not a requirement for disordered breathing. Therefore, a determination that a patient is asleep (for example, according to measured brain activity as detected by an electroencephalogram [EEG] sensor) is not required in order to provide disordered breathing therapy. However, stimulation therapy for disordered breathing causes the least discomfort to patients when they are sleeping and in many cases disordered breathing is most likely when a patient is sleeping. Thus, proxy signals for sleep, such as patient movement, patient pitch, patient activity, time of day, and so forth, as discussed herein, may be used as a surrogate for sleep detection, for example based on measured brain activity, because it is sufficient to determine that a patient is in a state in which they are most likely to experience disordered breathing and to tolerate higher and more efficient levels of stimulation energy. In other words, the likelihood of sleep as indicated by proxy signals is sufficient to support a provision of therapy. Patient movement and/or position data enables the system as described herein to evaluate a patient's relative tolerance for a particular level of stimulation energy and then titrate the stimulation energy based on that relative tolerance. The wakefulness evaluation based on patient position and/or movement may serve as a proxy for the tolerance evaluation. Such movement and/or position data may not detect sleep per se, for example in the same manner as an EEG measurement. However, in some cases, there may be a correlation between the wakefulness, or degree of arousal, of a patient and their relative tolerance.

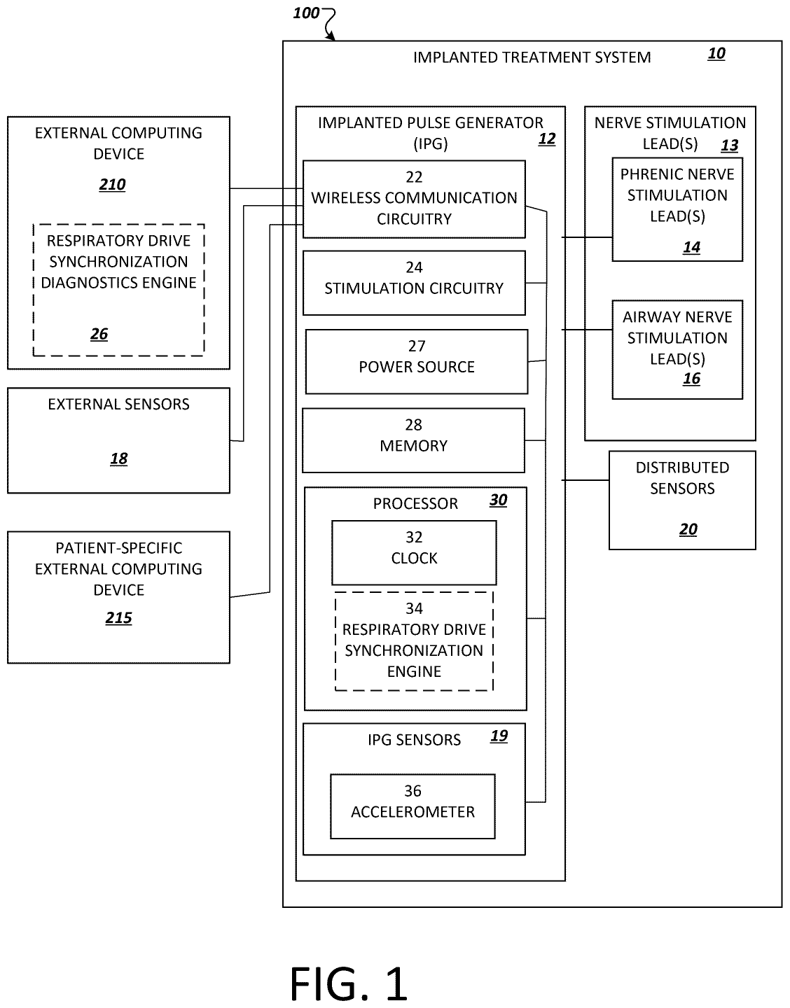

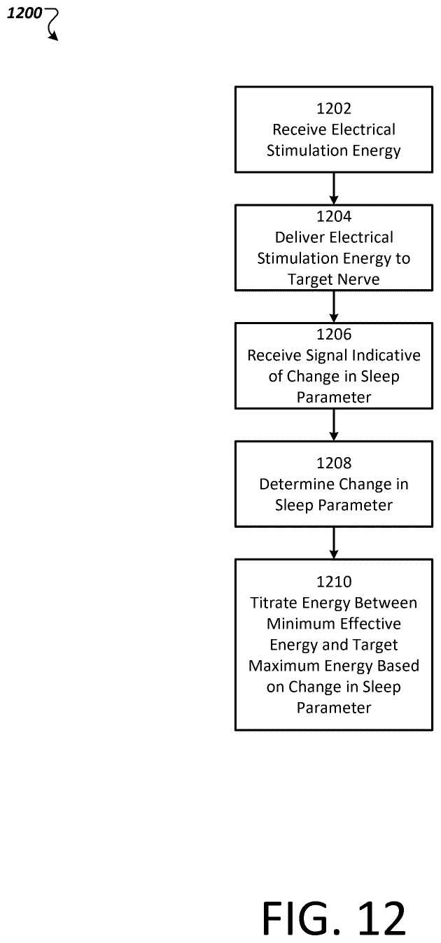

FIG. 1 illustrates a block diagram of a disordered breathing therapy system 100 according to an example. A quantity of each component in FIG. 1 is an example only and other quantities of each, or any, component could be used. The disordered breathing therapy system 100 may be implemented in connection with a patient to treat disordered breathing that occurs while the patient is sleeping, such as one or more forms of sleep apnea. For example, the disordered breathing therapy system 100 may be implemented in connection with a patient experiencing CSA events, OSA events, or other types of breathing disorders. FIG. 1 is described in part below and in part following the description of FIG. 12.

The disordered breathing therapy system 100 includes an implantable treatment system 10, an external computing device 210, one or more external sensors 18, and a patient-specific external computing device 215. The implantable treatment system 10 includes an implantable device 12 (for example, an implantable electrical pulse generator 12, or IPG 12) and one or more nerve stimulation leads 13.

The IPG 12 may include IPG sensors 19, wireless communication circuitry 22, electrical stimulation circuitry 24, at least one power source 27, memory 28, and at least one processor 30. The processor 30 includes a clock 32 and may optionally include a respiratory drive synchronization (RDS) engine 34. The IPG sensors 19 may include at least one accelerometer 36. The wireless communications circuitry 22 may include an electromagnetic telemetry transceiver along with at least one antenna for telemetry-based data communication and programming. Alternatively or additionally, the wireless communications circuitry 22 may include a transmitter, receiver, and/or transceiver configured for wireless communications, for example, according to a Bluetooth® protocol or other short-range communications protocol. In various implementations, the electrical stimulation circuitry 24 and the at least one processor 30 may support single channel operations to support stimulation of only one of one or more upper-airway nerves or one or more phrenic nerves. Alternatively, the electrical stimulation circuitry 24 and the at least one processor may support dual channel operations to support stimulation of a combination of at least one upper-airway nerve and at least one phrenic nerve.

The treatment system 10 is configured to be communicatively coupled to the computing system 210 and/or the patient-specific external computing device 215 via a wireless communication connection. Wireless communications may be exchanged pursuant to a short-range wireless communication protocol such as, for example, the Bluetooth® wireless communication protocol, or inductive telemetry, or another telemetry method. The computing system 210 may program and/or configure the IPG 12.

The IPG 12 may receive programmable operating parameters via software and/or firmware downloaded to the IPG 12 via wired or wireless communications. In an implementation, the external computing device 210 may generate the programmable operating parameters, for example, based at least in part on user input to the external computing device 210. These programmable operating parameters may include stimulation energy and/or energy titration parameters. For an implantable IPG, various electrical circuitry components may be enclosed in a hermetically sealed casing to protect these components from the body environment. The implantable IPG may further include a header disposed on the casing. The header may be a structural component of the implantable IPG that provides one or more receptacles, or connector ports, configured to receive respective connector assemblies for leads connected to electrodes. Each lead may couple with one or more connector ports. The header thus provides paths to electrically and mechanically couple the leads to the enclosed components of the implantable IPG 12 without comprising the hermetic seal. The connector ports may be configured and/or re-configured as sensor ports, stimulation ports, sensor-and-stimulation ports, and so forth according to programmed configuration settings provided to the IPG 12 through the firmware and/or software. The firmware and/or software may configure the connector ports to control and/or designate various electrodes as anodes, cathodes, and/or anode-cathode pairs.

The power source 27 provides power to one or more components of the implantable treatment system 10. The power source 27 may include an energy-storage device, such as a battery. In some examples, the power source 27 may be rechargeable. For example, the power source 27 may be configured to be wirelessly recharged by a wireless charger outside of the patient's body.

In various examples, the processor 30 is configured to process information and control operation of the treatment system 10, such as by controlling the delivery of electrical stimulation, for example, as a stimulation pulse train, to a target nerve. The processor 30 may include one or more processors to execute instructions stored on one or more non-transitory computer-readable media, such as the memory 28. Accordingly, when the treatment system 10 is described as determining information or parameters, executing operations such as comparing values to thresholds, modifying parameters for electrical stimulation, sensing or obtaining parameters or information, and so forth, the processor 30 may be performing these operations.

The memory 28 may include one or more memory devices, one or more storage devices, or a combination thereof. For example, the memory 28 may include one or more non-transitory computer-readable media. The one or more non-transitory computer-readable media may store instructions that, when executed by the processor 30, cause the processor 30 to execute operations discussed herein, such as titrating a stimulation energy. The memory 28 may also store other information, such as sensed data, user preferences, control commands from a physician, and so forth.

In operation, an electrical stimulation signal (for example, a pulse train) generated by the IPG 12 travels along the leads 13. The leads 13 include electrodes to deliver electrical stimulation to a target nerve. An electrical pulse train provided for a period of time to an electrode results in electrical stimulation of the nerve or other target tissue. The energy and the duration of the period of time determine the stimulation power provided to the target tissue. The electrical stimulation activates the fibers (that is, generates an action potential) causing a functional response by the nerve. A higher energy stimulation may activate more nerve fibers resulting in a larger functional response. A lower energy stimulation may activate fewer nerve fibers resulting in a smaller functional response.

For a particular functional response, such as the inducement of the contraction of a muscle (for example, the diaphragm), a minimum threshold energy may be required. The strength of the functional response (for example, the strength and/or extent of a muscular contraction needed to achieve a desired effect on respiration) determines the minimum threshold energy. In order to provide a range of functional responses (for example, a range of strengths and/or extents of muscular contractions to create a range of effects on respiration), a device, such as the IPG 12, may be configured to provide electrical stimulation to satisfy various energy thresholds. For example, there may be weak, medium, and strong thresholds to describe a range of functional responses.

As discussed in greater detail below, the disordered breathing therapy system 100 may include a respiratory drive synchronization (RDS) engine 34 and a respiratory drive synchronization (RDS) diagnostics engine 26. As shown in FIG. 1, the implantable treatment system 10 may include the RDS engine 34 and the external computing device 210 may include the RDS diagnostics engine 26. For example, the processor 30 may include or execute the RDS engine 34.

In an implementation, the implantable treatment system 10 may include the RDS diagnostics engine 26 in addition to or in lieu of the external computing device 210. The RDS diagnostics engine 26 may provide initial programming of the RDS engine 34 during an initialization phase of use of the system 100 with the patient (for example, in physician office visits designed to provide initial set-up of the implantable device 12). The RDS engine 34 may provide respiratory drive therapy as described herein and may modify respiratory drive treatment parameters based on RDS data to improve or otherwise modify synchronization. In some examples, the RDS diagnostics engine 26 may periodically update or re-program the RDS engine 34 (for example, during follow-up visits in a physician's office).

The engines 34 and 26 may include hardware (for example, one or more controllers configured to execute computer-executable instructions), software (for example, instructions stored on non-transitory computer-readable media that, when executed by hardware such as the one or more controllers, causes the treatment system 10 to deliver therapy), or a combination thereof. In some examples, the actions performed by engine 34 and/or 26 may be implemented by hardware, software, firmware, microcode, hardware description languages, and so forth, or any combination thereof. When implemented in software, firmware, or microcode, the program code or code segments to perform the tasks may be stored in a non-transitory processor-readable medium such as a storage medium. One or more processors, including the processor 30, may perform the described tasks.

The processor 30 may also include a clock 32. The clock 32 may be configured to identify a time of day, day of the week, month of the year, or another time indicator or clock information. In an implementation, the IPG 12 may use the clock information to control delivery of electrical stimulation signals to the leads 13.

The IPG 12 may receive sensed signals from one or more sensors, such as the external sensors 18, the IPG sensors 19, and/or the distributed sensors 20. Each of the sensors 18, 19, and/or 20 may include any type and/or number of sensors. For example, the IPG sensors 19 may include at least one accelerometer 36. The sensors 18, 19, and/or 20 may include sensors such as motion sensors (for example, accelerometers), pressure sensors, acoustic sensors, transthoracic-impedance sensors, light sensors, airflow sensors, microphones, ultrasonic transducers, heart rate sensors, blood oxygenation sensors (for example, pulse oximeters), muscular electrical activity sensors (for example, electromyography sensors), peripheral arterial tone sensors, or other types of sensors. In some examples, one or more of the leads 13 may include and/or act as sensors. Implanted and/or external accelerometers may be single axis accelerometers, or may be two- or three-axis accelerometers.

The external sensors 18 are external to the patient (that is, not implanted). The external sensors 18 may be wearable or otherwise coupled to the patient. The external sensors 18 may be communicatively coupled to one or more of the implantable device 12 or the external computing device 210 and may be electrically coupled to the external computing device 210. The external sensors 18 may provide sensed information to the IPG 12. For example, the external sensors 18 may include transthoracic impedance sensors configured to measure an impedance across a patient's thorax. The transthoracic impedance sensor data may be indicative of, for example, chest size, chest motion (that is, changes in chest size), cardiac parameters, and/or respiratory parameters that may be indicative of a degree of arousal.

The IPG sensors 19 may include one or more sensors on or within the IPG 12. The IPG sensors 19 may include at least one accelerometer 36. The at least one accelerometer 36 is an implantable accelerometer as opposed to the external motion sensor(s) 256 and/or the effort belt 254 which may include at least one external accelerometer located on the outside of the patient's body. For example, the at least one accelerometer 36 may be housed within the IPG 12. The at least one accelerometer 36 may be configured to sense a position and/or movement of a patient. The IPG sensors 19 may be implanted within a patient, at least because the IPG 12 may be implanted within a patient. Because the at least one accelerometer 36 may be implanted within a patient, the processor 30 may use the position and/or movement information sensed by the at least one accelerometer 36 to determine, for example, a position of a sleeping patient and may determine when a patient rolls from one position to another. As discussed below, this movement and/or position information may be used to modify therapy delivered to a patient at least because movement may correlate to a degree of arousal, or wakefulness, of the patient.

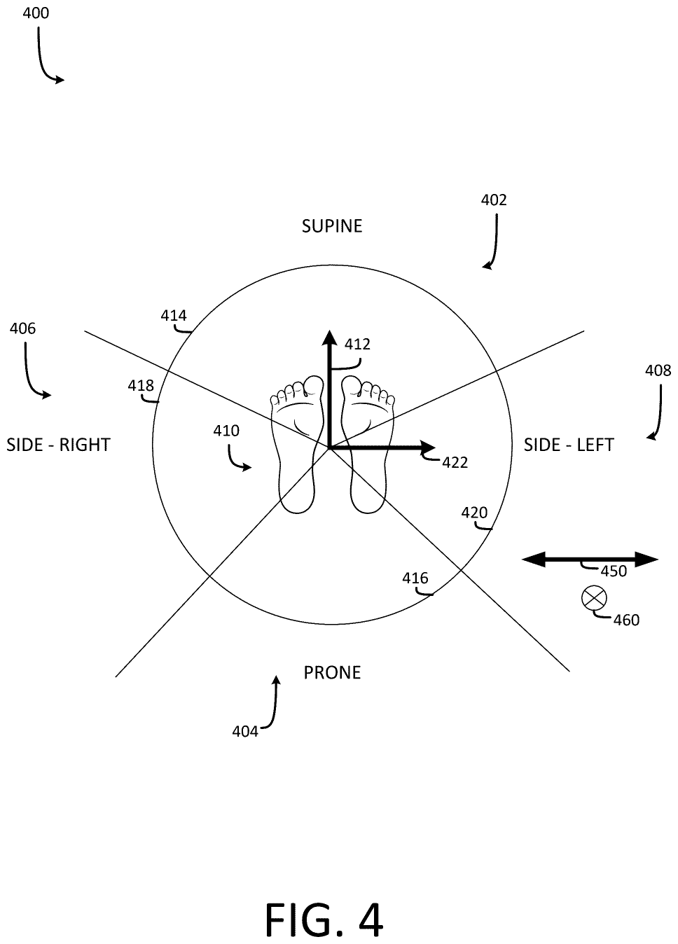

Acceleration data from the at least one accelerometer 36 may be calibrated according to positional quadrants (for example, the positional quadrants shown in FIG. 4) to account for a pitch of the accelerometer due to its implanted position in the patient and the body habitus of the patient (for example, chest size and/or geometry). For example, this may account for differences in data received from a barrel-chested patient as opposed to a thin-chested patient. The at least one accelerometer 36 may have a gravity reference and the IPG 12 may filter the signals from the at least one accelerometer to remove direct current (DC) offset.

The distributed sensors 20 may include one or more sensors electrically coupled to the IPG 12, but not physically located on or within a housing of the IPG 12. For example, the distributed sensors 20 may include one or more pressure sensors implanted within a patient. Pressure sensors implanted within a patient may measure changes in pressure caused by a patient breathing as the patient's lungs expand and contract, or may measure changes in pressure caused by a patient's heart beating as the patient's heart expands and contracts, or may measure other changes in pressure. The distributed sensors 20 may include at least one accelerometer. In an example, an accelerometer on a lead proximate to the hypoglossal nerve, or located elsewhere proximate to the airway, may detect motion indicative of airway patency. The distributed sensors 20 may be electrically coupled to the IPG 12 to provide sensed information to the IPG 12. In some examples, the distributed sensors 19 may include the one or more of the leads 13, whereas in other examples, the sensors 20 may be separate from the leads 13.

The IPG sensors 19 and/or the distributed sensors 20 may include transthoracic impedance sensors. Transthoracic impedance data may be indicative of, for example, chest size, chest motion (that is, changes in chest size), cardiac parameters, and/or respiratory parameters that may be indicative of a degree of arousal and/or patient motion. For example, patient motion may include a patient activity like coughing or snoring that correlate with a change in degree of arousal. As another example, the transthoracic impedance data may be indicative of a change in lung volume due to changes in the respiratory pattern of a patient caused by a change in patient position. Thus, while the transthoracic impedance may not directly measure patient motion in the manner of an accelerometer, the transthoracic impedance may serve as a proxy or predictive measurement of patient motion.

The external computing device 210 may include, for example, a computing device operated by a physician. A physician may use the external computing device 210 to control and/or program the implantable treatment system 10. For example, the physician may use the external computing device 210 to control and/or program the RDS engine 34 to modify parameters of therapy delivered by the IPG 12, such as a minimum treatment energy, a maximum treatment energy, a number of pulses in a pulse train, a frequency of pulses, a width of the pulses, a pulse amplitude, a maximum pulse amplitude, a stimulation current, a stimulation voltage, a stimulation polarity (for example, monophasic or biphasic), stimulation energy ramps, and so forth.

The patient-specific external computing device 215 may include, for example, a computing device operated by a patient in whom the implantable treatment system 10 is implanted. For example, the patient-specific external computing device 215 may include a smartphone or laptop operated by a patient. A patient may use the patient-specific external computing device 215 to communicate with the IPG 12, such as to view or adjust treatment parameters. In some examples, the patient-specific external computing device 215 may only be allowed to view information, but cannot be used to modify treatment parameters. For example, only the external computing device 210, which may be operated by a physician, may be used to modify treatment parameters in some examples.

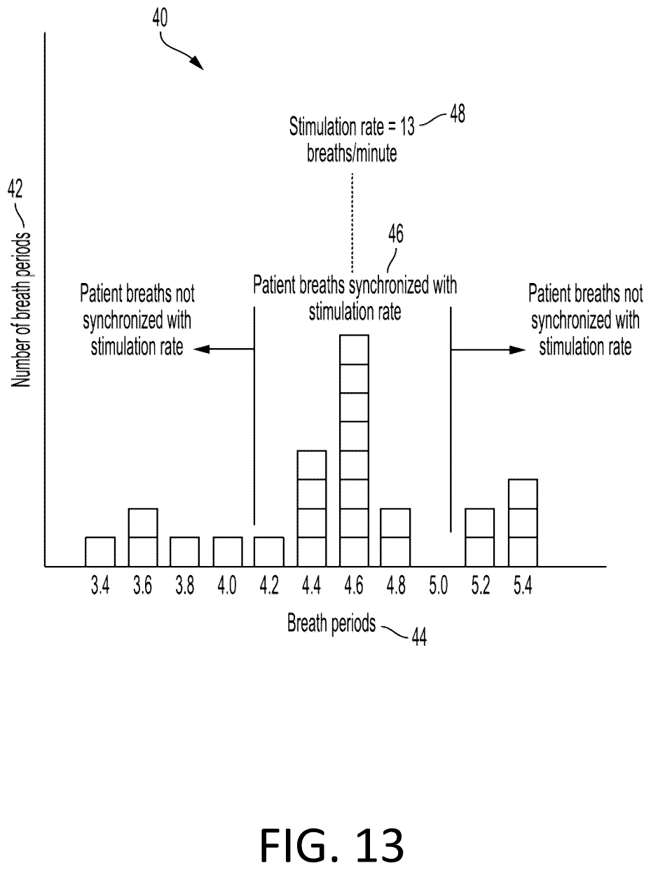

Stimulation of the phrenic nerve (for example, stimulation of the right phrenic nerve or the left phrenic nerve by the IPG 12) to produce rhythmic contractions of one hemidiaphragm innervated by the stimulated nerve and consequent rhythmic lung inflations may synchronize the patient's respiratory drive with the stimulation. When the patient's respiratory drive is synchronized in this manner, the patient may be referred to as entrained. With entrainment, the patient's breathing rhythm is modulated by the stimulation, becoming more regular and exhibiting less disorder. Indicators of synchronization are a respiratory rate of the patient that is clustered at or near the programmed rate of the stimulation. In addition, the contralateral hemidiaphragm may move in synchronization with the stimulated hemidiaphragm. Unilateral stimulation of a hemidiaphragm may provide some improvements in oxygenation and other respiration and/or sleep metrics even without synchronization. However, the unilateral stimulation with synchronization provides more substantial improvements in respiratory function. Thus, synchronization is a beneficial and important goal for disordered breathing therapy. With entrainment, spontaneous breathing and central respiratory drive are preferably preserved. Thus, an ordered rhythmic breathing pattern is established without a patient becoming dependent on the phrenic nerve stimulation. The patient is thus said to be “entrained” as opposed to “paced” by the nerve stimulation. Entrainment is discussed further in regard to FIG. 13 below.

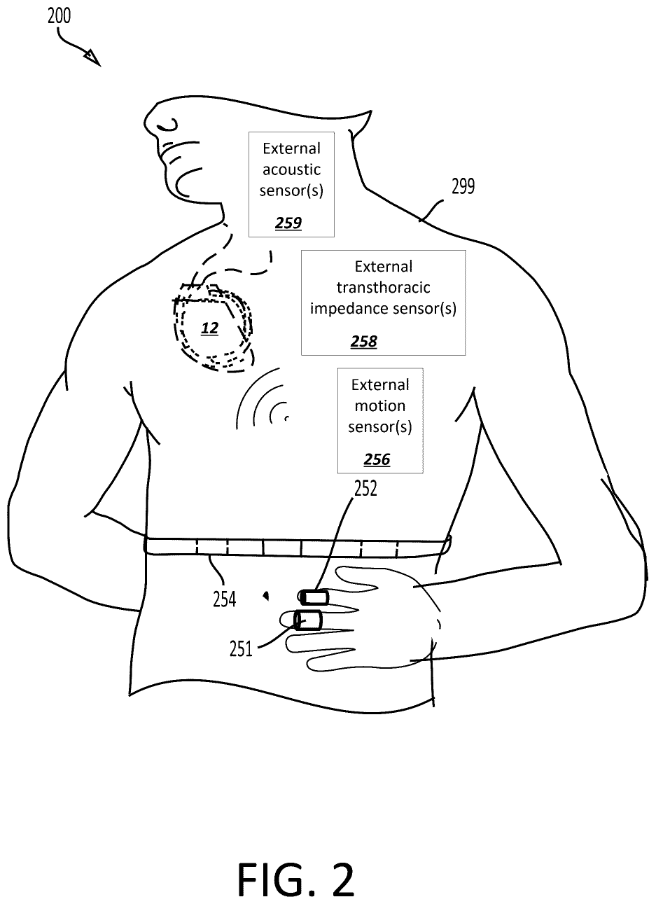

FIG. 2 illustrates a perspective view of a disordered breathing therapy system 200 using external sensors according to an example. A quantity of each component in FIG. 2 is an example only and other quantities of each, or any, component could be used. The disordered breathing therapy system 200 may be an example of the disordered breathing therapy system 10 in an implementation including one or more external sensors 18. For example, the one or more external sensors 18 may include a pulsatility sensor 252, an effort belt 254, external motion sensor(s) 256, external transthoracic impedance sensors 258, and/or external acoustic sensor(s) 259, and so forth.

The external motion sensor(s) 256 may include at least one external accelerometer. In various implementations, the external motion sensor(s) 256 may be affixed to the body of the patient at one or more locations (for example, chest, torso, neck, head, legs, arms, and so forth). These external motion sensor(s) 256 may detect patient activity. Data from the one or more external accelerometers included in the external motion sensor(s) 256 may be calibrated according to positional quadrants (for example, the positional quadrants shown in FIG. 4) to account for a pitch of the accelerometer due to its position on the patient. Further, the external motion sensor(s) 256 may have a gravity reference and the IPG 12 may filter the signals from the external motion sensor(s) 256 to remove direct current (DC) offset. The at least one external accelerometer may measure vibrations of a patient's tissues. The effort belt 254 may also include one or more external accelerometers.

In various implementations, the effort belt 254 is implemented to detect breathing of a patient 299, and the at least one pulsatility sensor 252 is implemented to measure pulsatility parameters, such as pulse rate, heart rate, pulse amplitude fluctuations, a peripheral arterial tone, and/or SpO2, and so forth. The external sensors 18 may include a pulse oximeter 251.

The implantable device 12 may also be coupled to one or more of the effort belt 254, the at least one transthoracic impedance sensor 258, and/or the at least one pulsatility sensor 252. The pulsatility sensor 252, the effort belt 254, the motion sensor(s) 256, the at least one transthoracic impedance sensor 258, and/or the external acoustic sensor(s) 259 may be examples of the external sensors 18 (for example, as shown in FIG. 1). The external sensors 18 may be communicatively coupled to the implantable device 12 in some examples, either directly (for example, via a wireless communication connection with the implantable device 12) or indirectly (for example, by communicating with the computing system 210 which, in turn, communicates with the implantable device 12).

The effort belt 254 may be coupled around the chest of the patient 299. As the patient 299 breathes, the effort belt 254 may deform. The effort belt 254 may include one or more pressure and/or stretch sensors to detect changes in pressure applied to the effort belt 254 and/or stretching of the effort belt 254 as a result of the patient 299 breathing. Accordingly, the effort belt 254 may sense parameters indicative of the breathing of the patient 299 which may be used to determine information such as a breathing rate of the patient 299.

The at least one transthoracic impedance sensor 258 may be coupled to, and external to, a chest of the patient 299. The at least one transthoracic impedance sensor 258 may sense an impedance across the chest of the patient 299. The measured impedance may vary based on the size and/or dimensions of the chest cavity of the patient 299 which may, in turn, vary as the patient 299 breathes. Accordingly, the at least one transthoracic impedance sensor 258 may sense parameters indicative of the breathing of the patient 299 which may be used to determine information such as a breathing rate of the patient 299. Transthoracic impedance data may be indicative of, for example, chest size, chest motion (that is, changes in chest size), cardiac parameters, and/or respiratory parameters that may be indicative of a degree of arousal and/or patient motion. For example, patient motion may include a patient activity like coughing or snoring that correlate with a change in degree of arousal. As another example, the transthoracic impedance data may be indicative of a change in lung volume due to changes in the respiratory pattern of a patient caused by a change in patient position. Thus, while the transthoracic impedance may not directly measure patient motion in the manner of an accelerometer, the transthoracic impedance may serve as a proxy or predictive measurement of patient motion.

The at least one pulsatility sensor 252 may be coupled to a finger of the patient 299. The at least one pulsatility sensor 252 may track pulsatility parameters of the patient 299, such as pulsatile volume changes in peripheral arterial beds as the heart of the patient 299 beats and/or as the patient 299 breathes. Accordingly, the at least one pulsatility sensor 252 may sense parameters indicative of the breathing of the patient 299 which may be used to determine information such as a breathing rate, heart rate, blood-oxygen concentration, and so forth, of the patient 299. With regard to the effort belt 254, in an implementation, the IPG 12 may use motion data from the belt 254 to assess entrainment, presence of respiratory effort, respiratory rate, and so forth. Based on this assessment the IPG 12 may initiate, terminate, reduce, or increase stimulation to the phrenic nerve stimulation leads 14 and/or the airway nerve stimulation leads 16 to titrate the treatment provided to the patient 299. Similarly, the IPG 12 may assess data from the transthoracic impedance sensor(s) 258, the external motion sensor(s) 256, and/or the pulsatility sensor(s) 252 in order to initiate, terminate, reduce, or increase stimulation to the one or more of the leads 13 to titrate the treatment provided to the patient.

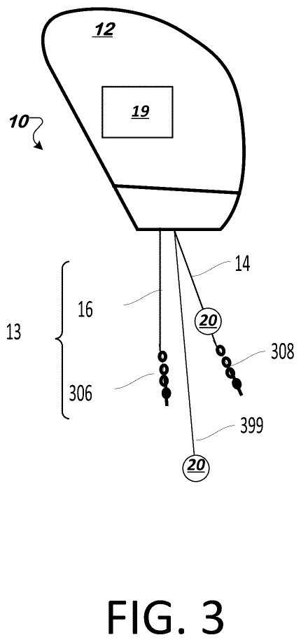

FIG. 3 illustrates an example of implantable components of a disordered breathing therapy system. A quantity of each component in FIG. 3 is an example only and other quantities of each, or any, component could be used.

As illustrated in FIG. 3, the implantable components 10 include the one or more leads 13 and the IPG 12. As discussed above, the leads 13 may include one or more phrenic nerve leads 14 and/or one or more upper airway stimulation leads 16. In these examples, therefore, the leads 13 may include at least two stimulation leads. Each lead includes at least one electrode (for example, the one or more electrodes 306 and 308). The electrodes 306 and/or 308 may be transvenous lead electrodes and/or nerve cuff electrodes. The electrodes 306 and/or 308 may serve as distributed sensors 20 and/or the stimulation leads may include distributed sensors 20. In some examples, the implantable components may include leads 399 without stimulation electrodes but that include sensor(s) 20.

The one or more phrenic nerve leads 14 may be configured for implantation in a blood vessel proximate to the phrenic nerve or may include a nerve cuff configured for implantation around the phrenic nerve. The one or more upper airway stimulation leads may be configured for implantation in a blood vessel proximate to the hypoglossal nerve or may include a nerve cuff configured for implantation around the hypoglossal nerve. Alternatively or additionally, the leads 13 may include lead configured for implantation in a blood vessel proximate the ansa cervicalis or other upper-airway nerve or may include a nerve cuff configured for implantation around the ansa cervicalis or other upper-airway nerve. Where a nerve has a left and right occurrence, the leads 13 may be configured to service one or both of the occurrences (for example, the left phrenic nerve, the right phrenic nerve, or both).

The IPG 12 may control the electrical energy delivered to the leads 13 to treat decreased respiratory drive (that is, to cause the diaphragm to contract due to stimulation of the phrenic nerve) and/or to improve or restore airway patency (that is, to cause movement of muscles in the upper airway due to stimulation of an upper-airway nerve such as, for example the hypoglossal nerve). For example, the IPG 12 may deliver electrical stimulation to one or more target nerves via the phrenic nerve stimulation lead(s) 14 and/or the airway nerve stimulation lead(s) 16. Such target nerves may include, for example, a phrenic nerve, a hypoglossal nerve, an ansa cervicalis nerve, other nerves that activate a diaphragm and/or stiffen a patient's airway, and so forth.

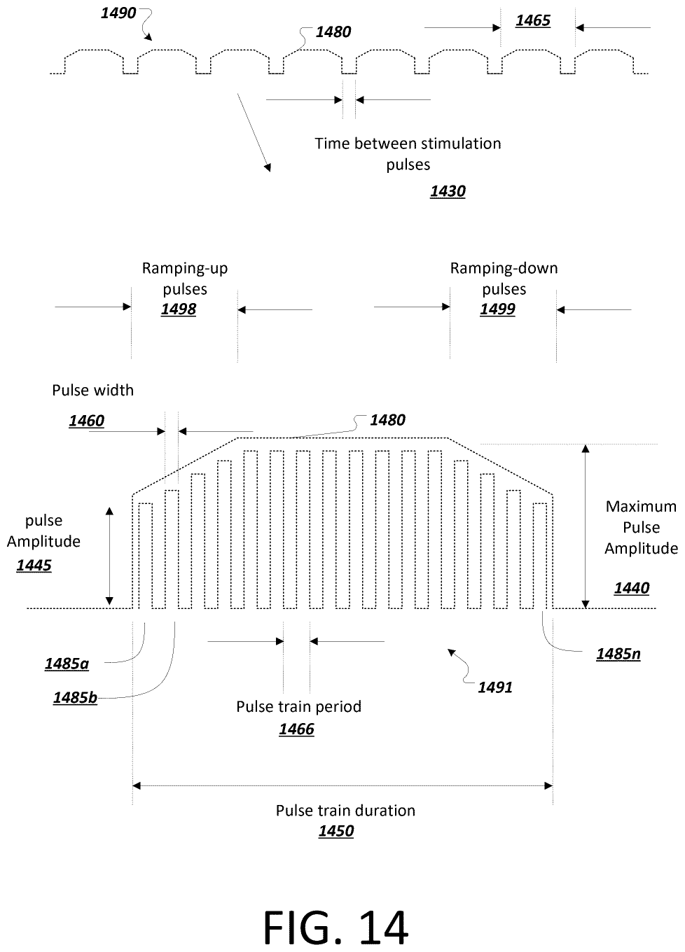

In various examples, the IPG 12 may deliver an electrical stimulation pulse train to one or more of the leads 13. An example of an electrical stimulation pulse train is shown below in FIG. 14. The leads 13 may each include electrodes to deliver electrical stimulation to a target nerve based on the electrical stimulation pulse train. For example, the processor 30 may control the stimulation circuitry 24 to provide the electrical stimulation pulse train to the leads 13.

The electrical stimulation pulse train delivered by the IPG 12 may be characterized by one or more electrical stimulation energy parameters. The IPG 12 may adjust one or more of these parameters to titrate the electrical stimulation energy delivered to a target nerve. Referring to FIG. 14, the electrical stimulation energy parameters may include parameters such as a pulse amplitude 1445, a maximum pulse amplitude 1440, a pulse period (or frequency) 1465, a pulse width 1460, a pulse train duration 1450, a stimulation current, a stimulation voltage, and a stimulation polarity (for example, monophasic or biphasic), stimulation energy ramps, and so forth.

One or more electrical stimulation energy parameters may be adjusted to titrate the stimulation energy delivered to the target nerve. Such titration may achieve a desirable balance between patient comfort and stimulation efficacy. For example, more intense electrical stimulation of a patient's phrenic nerve may yield higher activation of a patient's diaphragm, which may increase the efficacy of CSA treatment in some examples.

However, more intense electrical stimulation may also yield a greater sensory response from a patient. For example, a patient may feel more discomfort as the stimulation intensity is increased. Experiencing discomfort may adversely impact a patient's sleep. Even if a particular set of stimulation parameters effectively activate the patient's diaphragm, treatment according to that particular set of stimulation parameters may be contraindicated if the particular set of stimulation parameters causes the patient to wake up and lose sleeping time, for example due to discomfort produced by the particular set of stimulation parameters. It is therefore beneficial to the efficacy of stimulation therapy to identify stimulation parameters that produce a desired muscle response (for example, diaphragm contraction and expansion) while minimizing or avoiding patient discomfort.

In an implementation, the IPG 12 may ramp up the stimulation amplitude to increase the intensity of the stimulation, or may ramp down the stimulation amplitude to decrease the intensity of the stimulation. In general, the stimulation amplitude may vary over a range from a minimum amplitude for a pulse train to a maximum amplitude for the pulse train. The individual pulses within the pulse train may vary in amplitude but the maximum amplitude defines the maximum amplitude reached during a stimulation pulse train.

The IPG 12 may modulate the intensity of the stimulation if, for example, the patient is aroused by the electrical stimulation. The modulation of the intensity may depend on a degree of arousal. In various examples, the IPG 12 may determine the degree of patient wakefulness based on a patient's position or a change in a patient's position. For example, the patient's position may correspond to a positional quadrants. The change in the patient's position may correspond to rotational changes (for example, movement between positional quadrants), pitch changes, translation changes, and so forth.

The IPG 12 may determine a degree of arousal based at least in part on feedback information received from one or more of the sensors 18, 19, and/or 20. For example, the IPG sensors 19 include the at least one accelerometer 36. The at least one accelerometer 36 may be implanted in the patient's body (for example, in the patient's torso) such that movement sensed by the at least one accelerometer 36 may be used to determine a position and/or movement of the patient. The IPG 12 may determine patient position and/or movement based on sensed information from the at least one accelerometer 36.

The IPG 12 may use the position and/or movement information to determine if a patient is aroused. For example, if a patient rolls from one position (for example, a prone position) to another position (for example, a right-side position), then the IPG 12 may determine that the patient is being aroused by the stimulation and may decrease the intensity of the electrical stimulation. In some examples discussed in greater detail below, the IPG 12 may compare patient motion while stimulation is applied to patient motion when no stimulation is applied (that is, a baseline patient movement profile) such that deviations from the baseline may be identified as being caused by the stimulation.

The IPG 12 may be programmed to initiate disordered breathing therapy with a delivery of stimulation energy to one or more target nerves based on satisfaction of one or more sleep indication conditions. The sleep indication conditions may include detected movement of the patient. For example, movement detected by one or more accelerometers may provide movement data that may serve as a proxy for the actual sleep state of the patient. Patient activity, motion, position, and pitch as detected, for example, by an accelerometer, may be indicative of sleep particularly when combined with a time of day that corresponds to a regular sleeping time of the patient. Thus, the sleep indication conditions may further include pre-programmed stop and start times, a pre-programmed duration, and pre-programmed thresholds for patient pitch and patient movement. Optionally, the sleep indication conditions may include a patient position (for example, prone, supine, and so forth) and/or changes in physiological parameters, such as a reduction in heart rate. The IPG 12 may be pre-programmed to deliver therapy according to a regular schedule and may use the patient motion or position data to titrate that pre-programmed delivery.

For example, the IPG 12 may initiate therapy delivery when the pre-programmed sleep indication conditions are satisfied and modulate the intensity of the stimulation over a certain period of time after the sleep conditions have been satisfied for a threshold amount of time. In some examples, the IPG 12 may modulate the intensity of the stimulation based on pre-programmed parameters. In an example, upon initiation of therapy, the IPG 12 may increase the stimulation energy until the energy reaches a pre-programmed limit, at which point the IPG 12 may maintain a particular stimulation energy until the IPG 12 detects a change in one or more of patient wakefulness, sleep movement, and/or sleeping position. For example, if the detected change indicates that a patient's degree of arousal has increased, then the IPG 12 may reduce the intensity of therapy because the patient may experience more discomfort in a more wakeful state than in a less wakeful state.

In some examples, the IPG 12 may modulate the intensity of the stimulation based on feedback information indicative of the efficacy of the treatment in addition to, or in lieu of, modulating the stimulation energy based on pre-programmed parameters. Feedback information may include, for example, an entrainment index. As discussed above, an entrainment index may be determined based on a patient's breathing rate. The IPG 12 may receive information indicative of the patient's breathing rate from one or more of the sensors 18-20 and determine the entrainment index based on the patient's breathing rate. For example, the external sensors 18 may include a transthoracic-impedance sensor configured to send sensed information to the IPG 12 which may be used to determine the patient's breathing rate. In another example, the distributed sensors 20 may include at least one transthoracic-impedance sensor. For example, the distributed sensors 20 may include a lead that forms a cathode-anode pair with the housing of the IPG 12 itself. That is, the lead may be used to measure an impedance between the lead and the IPG 12, and this impedance may be a transthoracic impedance which may be used to determine breathing rate. In other examples, the sensors 18-20 may include other sensors to measure respiration.

In examples in which the IPG 12 receives feedback information indicative of patient respiration, the IPG 12 may modulate the energy of the stimulation based on the feedback information. Such modulation may be subject to an upper bound of stimulation energy as determined by a pre-programmed limit or as determined by an indication from the feedback information that the patient's respiration has reached a desired condition. Such a desired condition may include, for example, that the patient is successfully entrained.

FIG. 4 illustrates a diagram of a positional-quadrant system that may inform a determination of patient movement. The positional-quadrant system 400 includes a supine quadrant 402, a prone quadrant 404, a right-side quadrant 406, and a left-side quadrant 408. A sleeping position of a patient, whose feet 410 are depicted to indicate a direction 412 that the patient's body is facing, may be divided into one of the quadrants 402-408. The direction 412 that the patient's body is facing may therefore alternatively be referred to for purposes of simplicity as the patient position 412.

Each of the quadrants 402-408 spans a respective range of angular degrees, which may be equal in some examples and unequal in other examples. The supine quadrant 402 spans a supine range of angular degrees 414. The prone quadrant 404 spans a prone range of angular degrees 416. The right-side quadrant 406 spans a right-side range of angular degrees 418. The left-side quadrant 408 spans a left-side range of angular degrees 420.

Each of the ranges of angular degrees 414-420 may have various values, provided that the sum of the ranges of angular degrees 414-420 is 360°. In some examples, each of the ranges of angular degrees 414-420 is equal to 90°. In other examples, one or more of the ranges of angular degrees 414-420 is not equal to 90°. In some examples, the supine range of angular degrees 414 is equal to the prone range of angular degrees 416, and/or the right-side range of angular degrees 418 is equal to the left-side range of angular degrees 420. In other examples, one or more of the ranges of angular degrees 414-420 may not be equal to any other one of the ranges of angular degrees 414-420. A value for each of the ranges of angular degrees 414-420 may be specified by a programmer of the IPG 12, or by a physician on a patient-by-patient basis, or by the patient, and so forth.

In an example, the IPG 12 may detect patient movement based on patient rotational movement, or rolling, about an axis 460 that runs perpendicular to the page in FIG. 4. For example, a patient may roll from a supine to a side-left position with a rotational component about the axis 460.

In various examples, the IPG 12 may additionally or alternatively detect patient movement based on patient movement about a pitch axis 422. If the patient sits up, the patient may be rotating about the pitch axis 422. The at least one accelerometer 36 may sense this rotation. Patient movement information may indicate that the patient's position is rotating about the pitch axis 422.

In some examples, the IPG 12 may additionally or alternatively determine translation information for the patient. Translation information may include the patient's body moving through space in a manner other than rotating, although that movement may be accomplished by rolling. As one example, translation may be patient movement with a component along the translation axis 450. Translation may occur with the patient in any position based on quadrant. For example, a person on their back (supine) may translate across a bed surface or a person on their stomach (prone) may translate across a bed surface. Translation may occur in combination with other movement. For example, if a patient rolls 180° and moves two feet across a bed, the translation information is indicative of the two-feet movement across the patient's bed. Patient movement information may therefore include translation information in addition to, or in lieu of, rolling and/or pitch information.

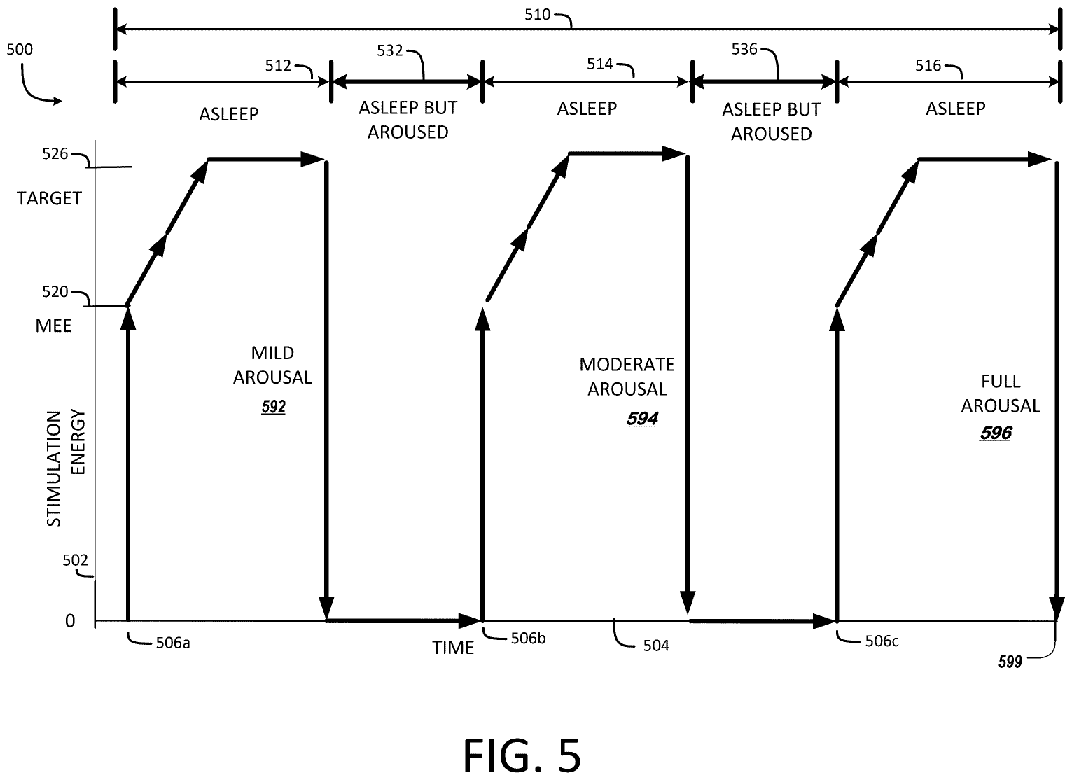

In order to illuminate the features of therapy titration responsive to modulations in wakefulness for a sleeping patient, FIG. 5 illustrates a simplified graph 500 depicting therapy titration independent of modulations in wakefulness for a sleeping patient.

FIG. 5 shows a mild arousal 592, a moderate arousal 594, and a full arousal 596. The patient is considered to be asleep for the entire time period represented in FIG. 5 prior to the time 599 after the full arousal 596.

The patient being considered to be asleep may be based on the satisfaction of pre-programmed sleep indication conditions as discussed above. Thus, the patient is able to best tolerate the delivery of stimulation therapy at least at a pre-determined and pre-programmed minimum effective energy (MEE) prior to the full arousal 596. As discussed herein, a patient referred to as asleep may tolerate the MEE when pre-programmed sleep indications are satisfied as determined by the IPG 12. When the patient is able to best tolerate the delivery of stimulation therapy at least at the MEE, this therapy is least likely to increase the patient's degree of arousal or wakefulness.

The higher state of arousal may correspond to a more wakeful state than the lower state of arousal. Additionally, the higher state of arousal may correspond to a lower patient tolerance for stimulation therapy. In the moderate arousal state 594, the patient can tolerate higher stimulation currents than in the mild arousal state 592. In the full arousal state 596, the patient is fully awake. Each arousal state may be characterized by one or more of a degree of patient movement, a type of patient movement, a patient position, a degree of patient activity, and/or a type of patient activity. In some examples, patient physical activity with little to no rotational, translational, or pitch movement may be indicative of a degree of arousal. For example, a patient may shake or jerk or move one or more limbs in a more wakeful state. As another example, a patient sitting up as indicated by pitch may indicate that the patient is in the full arousal state 596. Accordingly, if the patient's position rotates at least a threshold amount of angular degrees about the pitch axis 422 corresponding to the patient sitting up, the patient may be fully aroused and thus may be unable to tolerate the provision of stimulation energy.

In the example of FIG. 5, the IPG 12 increases the stimulation energy to at least a minimum effective energy (MEE) 520 and gradually increases up to a target energy 526 when pre-programmed sleep indications are satisfied as determined by the IPG 12. In this example, in response to any degree of arousal (for example, mildly, moderately, or fully aroused), the IPG 12 reverts to zero stimulation energy. This may occur even though the patient may still be sufficiently asleep (that is, mildly or moderately aroused) to tolerate at least the minimum effective energy and a continuation of nerve stimulation at a non-zero stimulation energy without undue discomfort.

The graph 500 includes a y-axis 502 and an x-axis 504. The y-axis 502 indicates a magnitude of energy provided by the IPG 12 to at least one of the leads 13 (and thus to one or more target nerves). The x-axis 504 indicates an elapsed time during a sleeping period of a patient. The elapsed time of the x-axis may or may not correspond to a full sleeping period starting at a pre-programmed state time and ending at a pre-programmed stop time. For example, the full arousal shown may be a patient awaking during the night to use the bathroom and then returning to sleep for another sleeping period. Alternatively, the full arousal shown may be a patient awakening after a night's sleep to get out of bed for the day. For purposes of discussion, a sleeping time period may be subdivided into various sleeping sub-intervals bounded by any of the patient being asleep 506a, 506b, 506c (that is, no degree of arousal detected based on patient motion and/or position), the patient being less than fully aroused (for example, mild arousal 592 or moderate arousal 594, both as indicated by patient motion and/or position) and the patient being fully aroused 596, as further indicated by patient motion and/or position.

For example, suppose that a patient falls asleep at 10 PM and awakens for the day at 7 AM and the pre-programmed start and stop times are 10 PM and 7 AM respectively. If the patient had no full arousals between 10 PM and 7 AM, then the sleeping period 510 would be from 10 PM to 7 AM. However, suppose that the patient briefly woke up at 1 AM before going back to sleep at 1:15 AM. Then sleeping period 510 would then be from 10 PM to 1 AM. Within any sleeping period, there may be one or more sub-intervals bounded by arousals that are not full arousals, for example, mild arousals or moderate arousals.

The graph 500 includes a sleeping time period 510, a first sub-interval 512, a second sub-interval 514, and a third sub-interval 516. During the sleeping time period 510, IPG 12 provides the stimulation energy to the target nerves as disordered breathing therapy during the first, second, and third sub-intervals. However, as a result of the reversion to zero stimulation energy the IPG 12 does not apply any stimulation energy during the zero stimulation energy periods 532 and 536. These periods may last for a pre-determined suspension window (for example, one minute), or may last until the IPG 12 determines, based on feedback information determined based on information from the sensors 18-20, that pre-programmed sleeping conditions are satisfied. Thus, although the patient is asleep during the periods 532 and 536, the stimulation energy is at zero due to a detected arousal.

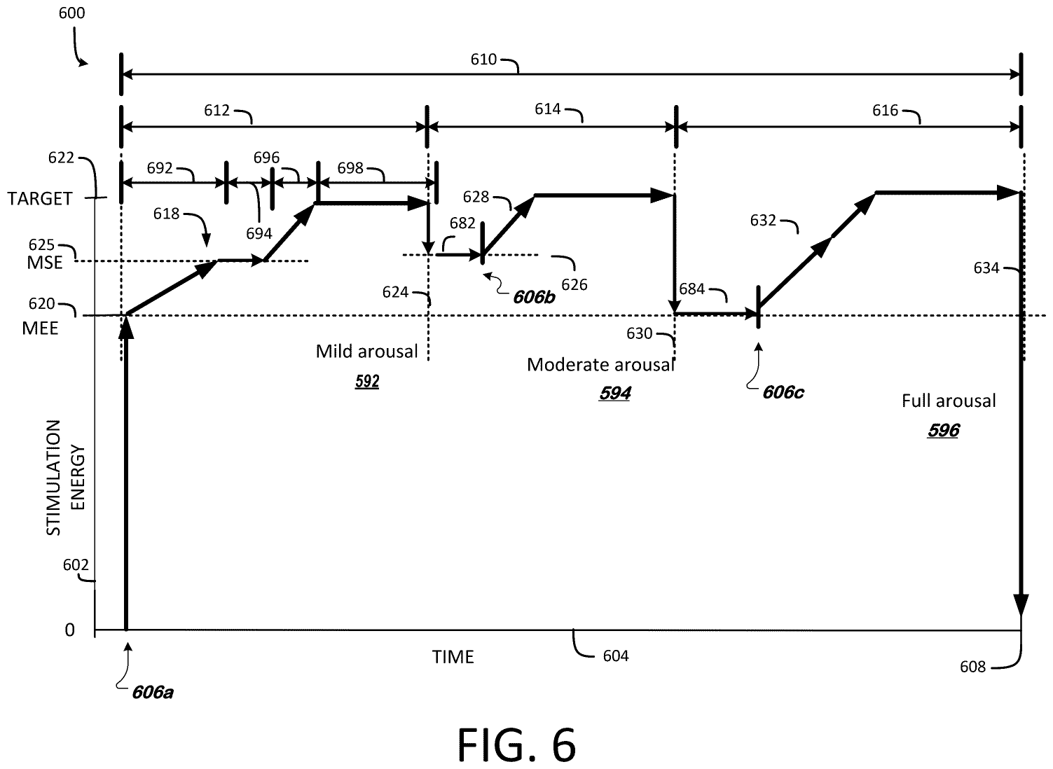

Example implementations of stimulation therapy titration based on a degree of arousal are discussed in greater detail below with respect to FIGS. 6-10B. Unlike FIG. 5, in these examples, the IPG 12 maintains a non-zero stimulation energy for all degrees of arousal other than a full arousal. Accordingly, such an approach may further improve the benefits to the patient of oxygen inhalation and carbon dioxide exhalation provided by the disordered breathing therapy. Additionally, such an approach reduces the amount of time during a sleeping period that the system is ramping energy back up to a target energy.

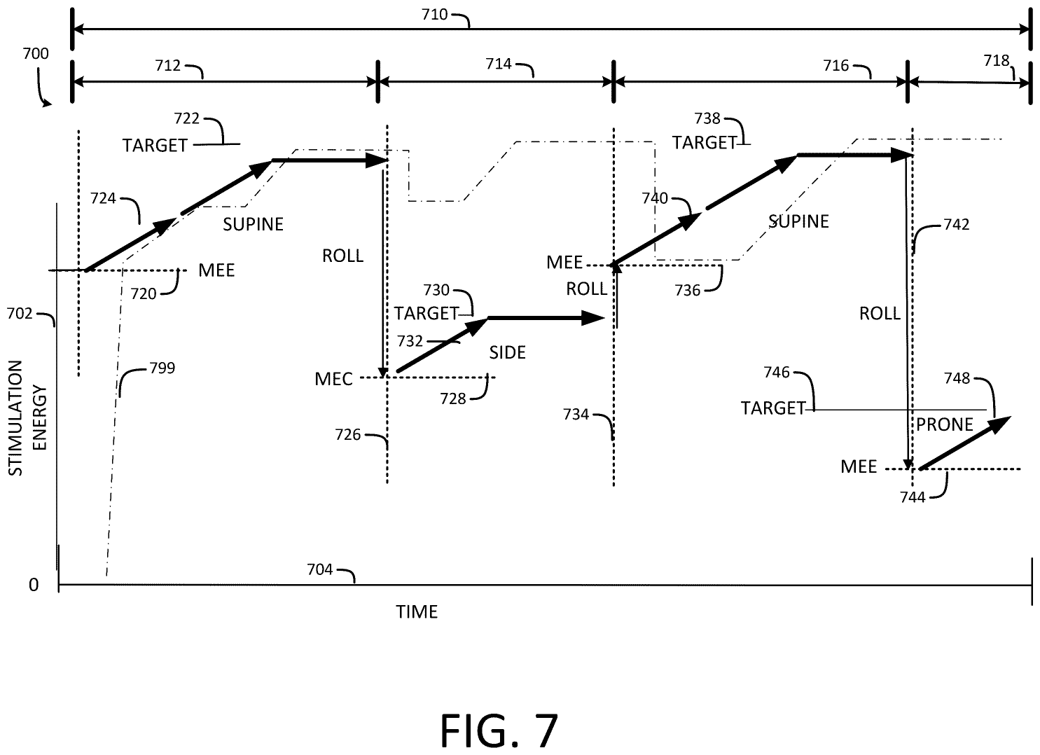

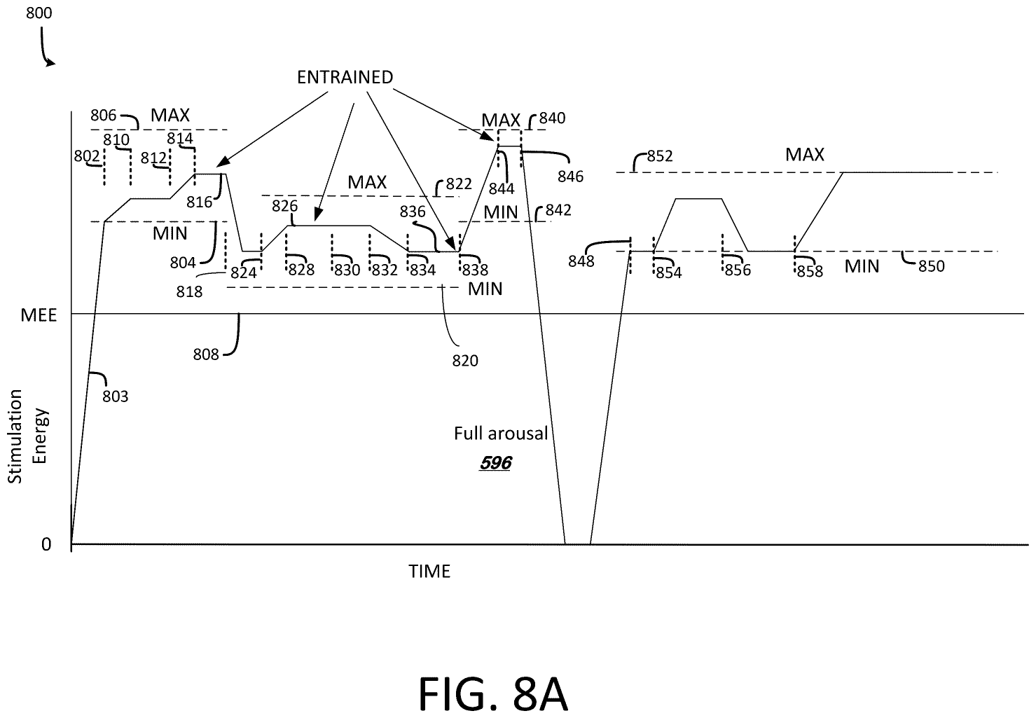

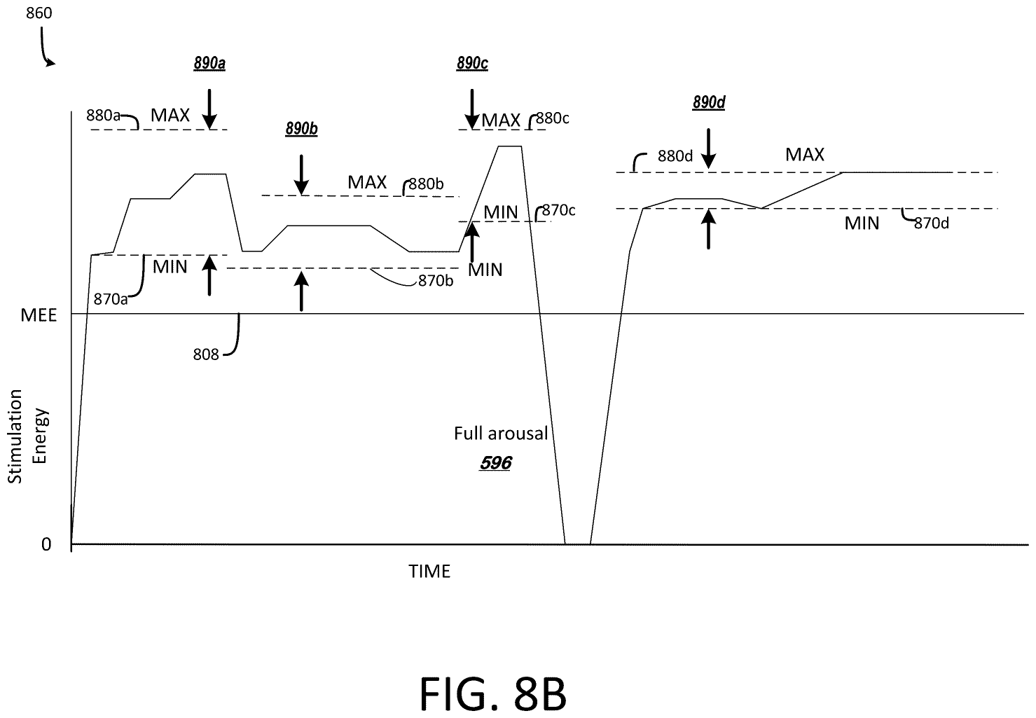

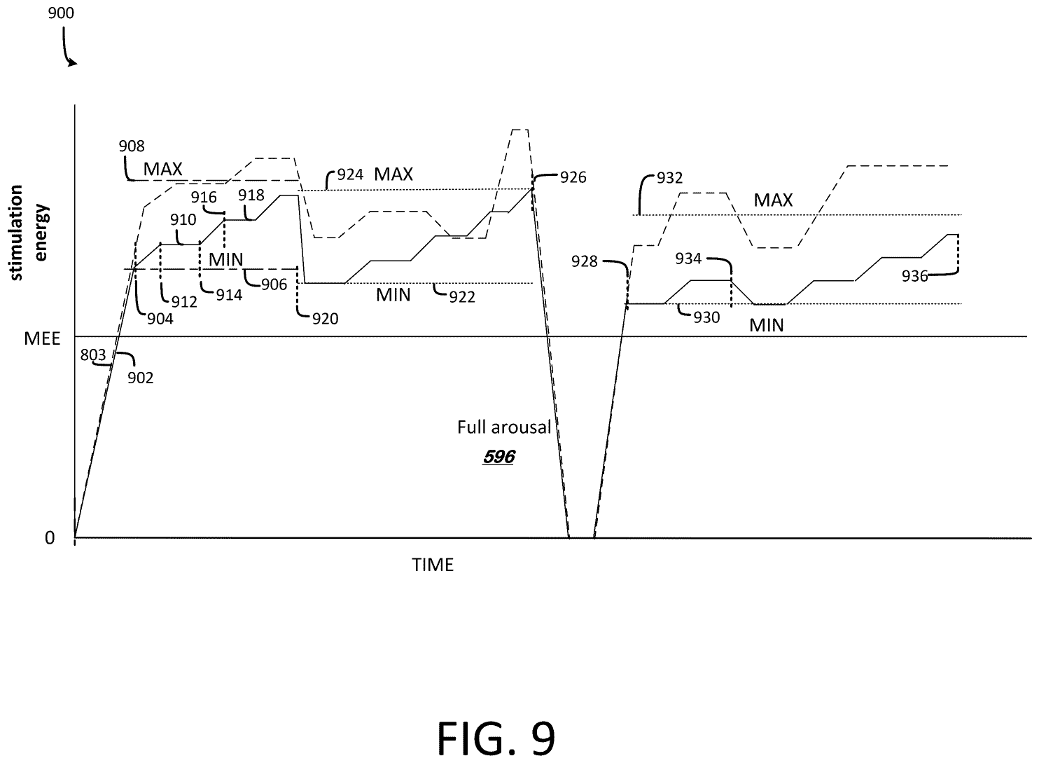

FIGS. 6 and 7 illustrate simplified graphs of electrical stimulation energy. FIGS. 6 and 7 are illustrated in part to explain how the IPG 12 may respond to patient arousal and/or position, and to introduce different minimum and maximum treatment energy values between which energy is titrated. However, FIGS. 6 and 7 provide simplified single energy traces for purposes of example. FIGS. 8A and 9 illustrate more detailed illustrations of stimulation-energy traces. In some examples, the IPG 12 may provide different stimulation currents to two or more of the leads 13 in combination. The different stimulation currents may have different values. While FIGS. 6 and 7 illustrate a single trace for purposes of explanation, in various examples the IPG 12 may be configured to be able to provide multiple different stimulation currents in combination. As illustrated in FIGS. 6 and 7, the processor 30 may cause the stimulation circuitry 24 to titrate a stimulation energy generated by the power source 27. The titration may include one or more adjustments between a target maximum treatment energy and a minimum treatment energy. In some examples, the minimum treatment energy is a minimum effective energy.

FIG. 6 illustrates a simplified graph 600 depicting therapy titration responsive to modulations in wakefulness for a sleeping patient according to an example. The graph 600 is simplified by showing the stimulation energy increasing linearly to a specific target energy. In various examples, however, the stimulation energy may be titrated to increase in a non-linear fashion to a target energy, which may change over time.

The graph 600 includes a y-axis 602 and an x-axis 604. The y-axis 602 indicates a magnitude of energy provided by the IPG 12 to at least one of the leads 13. The x-axis 604 indicates an elapsed time for a sleeping period 610. Similarly to FIG. 5, the sleeping period 610 includes a first sub-interval 612, a second sub-interval 614, and a third sub-interval 616. However, in contrast to FIG. 5, the stimulation energy does not revert to zero in response to a mild arousal 592 or a moderate arousal 594. The stimulation energy may only revert to zero in response to a full arousal 596. As a result, there are no zero-stimulation-energy periods like the periods 532 and 536 shown in FIG. 5. Additionally, because the stimulation energy shown in the example of FIG. 6 undergoes significantly fewer excursions to zero stimulation energy (for example, only for full arousals), the amount of time during a sleeping period that the system is ramping energy back up to a target energy is significantly reduced. The most benefit from therapy is provided at the target energy.

The IPG 12 may store a pre-programmed stimulation period that corresponds to a patient's usual sleep cycle. The patient being considered to be asleep may be based on the satisfaction of pre-programmed sleep indication conditions as discussed above. For example, a patient may generally sleep from 10 PM to 7 AM or from 10 AM to 6 PM. The IPG 12 for this patient would be pre-programmed (for example, by the external computing device 210) to provide disordered breathing therapy during a daily sleeping period 10 PM to 7 AM or the time period 10 AM to 6 PM. According to the example of FIG. 6, during this pre-programmed time period, the IPG 12 is configured to be able to provide the stimulation energy upon detection of sleep and stops the stimulation energy upon detection of full arousal. If the detection of full arousal precedes the end of the daily sleeping period (for example, before 7 AM or before 6 PM), then the provision of stimulation energy will resume upon detection of the patient again satisfying the pre-programmed sleep indication conditions.

The IPG 12 is configured to be able to then modulate or titrate the provision of stimulation energy thereafter according to a patient's level of arousal. The sleeping period 610 is within and may be less than or equal to the daily sleeping period pre-programmed into the IPG 12. In some examples, the sleeping period 610 may repeat two or more times within the daily sleeping period. However, the sequence of arousals and titration responses shown in FIG. 6 are examples only and not limiting of the disclosure. The specific pattern or sequence of arousal and titration will vary from patient to patient and may vary for one patient from day to day, week to week and/or within the daily sleeping period. Thus, the system described herein enables a dynamic response to a patient's particular arousals and sleeping patterns.

The first sub-interval 612 includes a first energy trace 618 depicting a magnitude of energy delivered by the IPG 12. The first energy trace 618 ramps up from a minimum effective energy (MEE) 620 to a target energy 622 upon detection of a patient being asleep at the time 606a. The stimulation energy is maintained at the target energy 622 until the IPG 12 determines that the patient is aroused at a first time 624.

The MEE 620 may include a minimum energy magnitude to modulate a patient's diaphragm and/or upper airway patency. The value of the MEE 620 may vary from patient to patient. Thus, this value is typically assessed for each patient, for example during observation by a physician and/or during overnight evaluation testing. The MEE 620 may also vary with sleeping position. Currents below the MEE 620 may not modulate the patient's diaphragm and/or upper airway patency and may thus not treat disordered breathing or sleep apnea. Accordingly, the IPG 12 may apply a stimulation energy no lower than the MEE 620 when delivering therapy. As illustrated in FIG. 6, if the patient experiences a full arousal 596, then the IPG 12 reverts to zero stimulation energy (for example, at the point 608).

The first energy trace 618 ramps up over the first ramp portion 692 of the first sub-interval 612. Although the first energy trace 618 is indicated as a straight line for ease of explanation, in various examples the magnitude of the energy indicated by the first energy trace 618 may increase in a stepwise fashion, as discussed in greater detail below. After the IPG 12 ramps up the stimulation energy from the MEE 620 to a maximum sub-interval energy (MSE) 625, the IPG 12 may optionally maintain the stimulation energy at the MSE 625 for a hold period 694. Thus, rather than continuously ramping up the stimulation energy over the first sub-interval 612, the IPG 12 may gradually increase the stimulation energy with hold periods during which the stimulation energy is maintained at a given magnitude.

The IPG 12 may determine an interval between the MSE 625 and the MEE 620 based on a magnitude of the target energy 622. The target energy 622 may be a maximum treatment energy that the IPG 12 ultimately ramps up to in a given sleeping interval 610. Whereas the MSE 625 is a maximum treatment energy for the first ramp portion 692 of the first sub-interval 612, the target energy 622 is a maximum treatment energy for the sub-interval 612 and the sleeping period 610 as a whole.

The IPG 12 may be pre-programmed with a maximum treatment energy based on an expected tolerance for the patient. For example, where the IPG titrates the treatment energy by adjusting stimulation current, the maximum treatment energy may correspond to a stimulation current between 5-20 mA, 5-10 mA, 2-6 mA, or 1-4 mA, depending on the target nerve, the type of electrode, and/or IPG battery conservation requirements. For example, the maximum treatment energy for a transvenous lead electrode may be greater than the maximum treatment energy for a cuff electrode. In some examples, the maximum treatment energy for a transvenous lead electrode correspond to a 6 mA stimulation current whereas the maximum treatment energy for a cuff electrode may correspond to a 4 mA stimulation current. The target energy 622 may be this pre-programmed maximum treatment energy value or may be a stimulation energy determined by patient entrainment.

In some examples, the IPG 12 may ramp up to the pre-programmed maximum treatment energy, regardless of whether or not the patient is entrained at a lower energy. In other examples, the IPG 12 ramps up to an energy at which the patient is entrained even if the energy is not yet at a pre-programmed maximum treatment energy value. For example, if a pre-programmed maximum treatment energy corresponds to 6 mA, but the patient is entrained at 4 mA, the IPG 12 may stop increasing the current at 5 mA. In this example, 6 mA achieves the target energy 622. In another example, if the pre-programmed maximum treatment energy value corresponds to a 6 mA stimulation current and the patient is still not entrained at 6 mA, the IPG 12 may nonetheless stop increasing the current at 6 mA because 6 mA corresponds to the pre-programmed maximum treatment energy value. In this example, 6 mA corresponds to the target energy 622 even though the patient may not be entrained.

After the first ramp portion 692 and the optional hold time 694, the first energy trace 618 continues to ramp up from the MSE 625 to the target energy 622 over a second ramp portion 696 of the first sub-interval 612. Once the first energy trace 618 reaches the target energy 622 (that is, once the IPG 12 is providing the maximum treatment energy corresponding to the target energy 622), the IPG 12 maintains the stimulation energy at the target energy 622.

After the second sub-interval 614, the IPG 12 maintains the stimulation energy at the target energy 622 for a maintenance duration 698 until the IPG 12 detects patient motion and determines a degree of arousal. For example, the IPG 12 may detect patient motion and determine the degree of arousal based on sensor data that may include patient movement data.

Example degrees of arousal include mild arousal, moderate arousal, and full arousal, as discussed above in regard to FIG. 5. The IPG 12 may determine the degree of arousal based on at least one sleep parameter, such as patient-movement information and/or patient-position information. A degree of arousal may be determined based on patient movement, such as positional changes and/or physical activity. For example, rotational position changes may correspond to a number of positional quadrants that the patient moves through in a given period of time. For example, if the patient rolls between two quadrants (for example, from the supine quadrant 402 to the left-side quadrant 408), then the patient may be considered mildly aroused. If the patient rolls between three quadrants (for example, from the supine quadrant 402 to the left-side quadrant 408 and then from the left-side quadrant 408 to the prone quadrant 404) in a given period of time (for example, within five seconds), the patient may be considered to be experiencing moderate arousal. If the patient rotates about the pitch axis 422 by at least a threshold number of angular degrees corresponding to the patient sitting up, then the patient may be considered to be fully aroused. These examples of degrees of patient arousal are non-limiting examples only; in other examples, other methods or parameters may be implemented to determine a degree of patient wakefulness.

In some examples, the rotations about the pitch axis may be subject to different thresholds. For example, a patient may sleep on a number of pillows such that their pitch is not indicative of a horizontal or near-horizontal body position. However, such a non-horizontal pitch may not be indicative of increased wakefulness. In addition, using various thresholds, in some examples, the IPG 12 may titrate therapy based on changes in pitch rather than based on a value of the pitch in order to accommodate non-horizontal or close to vertical sleeping positions, at least for the torso of the patient.

In some examples, patient arousal may be classified into one or more discrete groups, such as no arousal, mild arousal, moderate arousal, and full arousal. In other examples, patient arousal may be determined without discrete classifications. A degree of arousal may be represented by values such as a number of angular degrees rolled in a certain amount of time. An amount of patient arousal may correlate with a rate of change in the patient position 412 (that is, a number of angular degrees moved in a given time), but may or may not be classified into one or more discrete groups.

In any of the examples above, patient arousal may be determined by comparing a present patient movement to a baseline patient movement profile determined during a calibration phase before stimulation is applied and/or during an overnight study after stimulation therapy begins. For example, the stimulation therapy may be temporarily paused for such an overnight study to eliminate wakefulness changes due to the therapy itself. For example, if a patient movement deviates from the baseline patient movement profile by the patient rolling two positional quadrants, the patient may be considered mildly aroused. If the patient movement deviates from the baseline patient movement profile by the patient rolling three positional quadrants, the patient may be considered moderately aroused. In other examples, other deviations from a baseline patient movement profile may be characterized differently

At the first time 624, the IPG 12 determines that the patient is aroused. In the example of FIG. 6, the arousal at the first time 624 is shown as a mild arousal. The IPG 12 may determine a degree of patient wakefulness based on at least one sleep parameter, such as patient-movement or patient-position information. For example, the IPG 12 may determine a first degree of arousal of the patient based on patient-position or patient-movement information. In some examples, the first degree of arousal may include the patient rolling a certain number of positional quadrants. In various examples, the first degree of arousal may include the patient rolling a certain number of angular degrees in a certain amount of time. In at least one example, the first degree of arousal may indicate a degree of arousal compared to a baseline arousal of the patient before any stimulation is applied, which may be indicated by a baseline patient movement profile.

For example, the IPG 12 may receive patient-movement information from the at least one accelerometer 36 and determine, based on the patient-movement information, that the patient's position 412 has moved from one of the quadrants 402-408 to another of the quadrants 402-408 and thus identify a mild arousal. As another example, the IPG 12 may determine, based on patient-movement information received from the at least one accelerometer 36, that the patient's position 412 has moved through at least two of the quadrants 402-408 within a threshold period of time (for example, within five seconds) and thus identify a moderate arousal.

In some examples, arousal may be caused by the electrical stimulation. In other examples, the mild or moderate arousal may have been caused for some other reason, such as a loud noise or bright light disturbing the patient. However, with the patient in a mildly or moderately aroused state (whether due to the electrical stimulation or some other stimulus), the patient may be more susceptible to full arousal (for example, waking up) and/or discomfort if the IPG 12 continues to apply electrical stimulation at the present energy level. With a mild arousal, the patient may tolerate a higher electrical stimulation energy than in a moderate arousal.

In response to determining the first degree of arousal, the IPG 12 may decrease the stimulation energy. In some examples, the IPG 12 decreases the stimulation energy to the MEE 620 regardless of how aroused the patient is. In other examples, the IPG 12 decreases the stimulation energy to a value equal to or greater than the MEE 620 based on the degree of arousal. For example, the IPG 12 may decrease the stimulation energy based on how aroused the patient is (that is, based on the first degree of arousal). If the patient is more aroused (for example, if the patient meets at least the moderate arousal level), then the IPG 12 may reduce the stimulation energy by a larger amount (for example, to the MEE 620). If the patient is less aroused (for example, if the patient meets the mild arousal level), then the IPG 12 may reduce the stimulation energy to a value greater than the MEE 620.

For purposes of example, the IPG 12 may determine at the first time 624 that the patient is only slightly aroused. For example, the IPG 12 may determine that the patient has rolled a single positional quadrant (or has rolled a single additional positional quadrant beyond a baseline number of rolls) and is thus in a mild state of arousal. In some examples, the IPG 12 may have access to a baseline arousal profile indicative of baseline arousal parameters. The baseline arousal profile may be determined before any stimulation is applied, such that a difference between the baseline arousal profile and any arousal parameters acquired after stimulation is applied may be more accurately attributed to the electrical stimulation. In at least one example, a baseline arousal profile may include a baseline movement profile indicative of patient movement before any stimulation is applied. Determining that the patient is aroused at the first time 624 may therefore include determining not only that the patient is aroused, but that the first degree of arousal deviates from a baseline arousal by a slight amount. Examples of baseline arousal profiles are discussed in greater detail below.

In response to determining that the patient is slightly aroused at the first time 624, the IPG 12 may decrease the stimulation energy to an intermediate effective energy (IEC) 626. The IEC 626 is greater than the MEE 620. Accordingly, even though the IPG 12 has decreased the stimulation energy, the stimulation energy is still in an effective zone (for example, greater than or equal to the MEE 620). Decreasing the stimulation energy may therefore decrease patient arousal while maintaining effective treatment.

The end of the first sub-interval 612 may coincide with the beginning of the second sub-interval 614. Whereas the first sub-interval 512 and the second sub-interval 514 of FIG. 5 are separated by a period 532 during which no energy is applied, the first sub-interval 612 and the second sub-interval 614 may be contiguous with stimulation energy maintained at a level lower than the target 622 but higher than the MEE 620 (for example, the MSE 625). The IPG may maintain the lower level of energy for a time period 682 until the IPG 12 detects (for example, at 606b) that the patient is again asleep and once more able to tolerate the target energy 622.

The second sub-interval 614 includes a second energy trace 628 indicating an energy provided by the IPG 12. As indicated by the second energy trace 628, the IPG 12 ramps up the stimulation energy from the IEC 626 to the target energy 622. Because the IEC 626 is greater than the MEE 620, the IPG 12 may spend less time ramping up from the IEC 626 to the target energy 622 during the second sub-interval 614 than the IPG 12 spends ramping up from the MEE 620 to the target energy 622 during the first sub-interval 612. Once the stimulation energy reaches the target energy 622, the IPG 12 may maintain the stimulation energy at the target energy 622. The stimulation energy is maintained at the target energy 622 until the IPG 12 determines that the patient is aroused at a second time 630. As noted above, however, the target energy 622 may not be a constant value. For example, the actual value of the target energy 622 may decrease over time. As discussed in greater detail below, the IPG 12 may gradually reduce a stimulation energy if a desired level of efficacy is being achieved, such as if an entrainment index is at or above a threshold level. Accordingly, while the IPG 12 maintains the stimulation energy at the target energy 622, the actual value of the stimulation energy may change provided that entrainment is maintained in some examples. Upon detection of arousal at the second time 630, the IPG 12 may reduce the stimulation energy to a level below the target 622 but above or equal to the MEE 620. The IPG 12 may maintain this reduced level of stimulation energy for a time period 684 until the IPG 12 detects (for example, at 606c) that the patient is again asleep and once more able to tolerate the target energy 622.

At the second time 630, the IPG 12 determines that the patient is aroused. For example, the IPG 12 may determine a second degree of arousal of the patient based on patient-position or patient-movement information. In some examples, the second degree of arousal may include the patient rolling a certain number of positional quadrants within a certain period of time. In various examples, the second degree of arousal may include the patient rolling a certain number of angular degrees in a certain amount of time. In at least one example, the second degree of arousal may indicate a degree of arousal beyond a baseline amount of arousal where the baseline is determined for the patient before any electrical stimulation is applied.

For purposes of example, the IPG 12 may determine at the second time 630 that the patient is moderately aroused. For example, the IPG 12 may determine that the patient has rolled multiple positional quadrants (or multiple more positional quadrants than a baseline amount of rolls) in a certain period of time and is thus in a moderate state of arousal. Determining that the patient is aroused at the second time 630 may include determining not only that the patient is aroused, but that the second degree of arousal deviates from a baseline arousal by a moderate amount. Examples of baseline arousal profiles are discussed in greater detail below.

In response to determining that the patient is moderately aroused at the second time 630, the IPG 12 may decrease the stimulation energy to the MEE 620. Accordingly, even though the IPG 12 has decreased the stimulation energy, the stimulation energy is still in an effective zone (for example, equal to or greater than the MEE 620). Decreasing the stimulation energy may therefore decrease patient arousal while maintaining effective treatment.

The third sub-interval 616 includes a third energy trace 632 indicating an energy provided by the IPG 12. As indicated by the third energy trace 632, the IPG 12 ramps up the stimulation energy from the MEE 620 to the target energy 622. Once the stimulation energy reaches the target energy 622, the IPG 12 may maintain the stimulation energy at the target energy 622. The stimulation energy is maintained at the target energy 622 until the IPG 12 determines that the patient is aroused at a third time 634.

At the third time 634, the IPG 12 determines that the patient is aroused. For example, the IPG 12 may determine a third degree of arousal of the patient based on patient-position or patient-movement information. In some examples, the third degree of arousal may include the patient rolling a certain number of positional quadrants in a certain period of time. In various examples, the third degree of arousal may include the patient rolling a certain number of angular degrees in a certain amount of time, or pitching forward (for example, sitting up) by a certain number of angular degrees.

For purposes of example, the IPG 12 may determine at the third time 634 that the patient is fully aroused. For example, the IPG 12 may determine that the patient has pitched forward (for example, sat up) and is thus fully aroused (for example, awake). In response to determining that the patient is fully aroused at the third time 634, the IPG 12 may stop providing stimulation energy. Thus, only when the patient is fully aroused does the IPG 12 reduce the stimulation energy to below the MEE 620

As exemplified in FIG. 6, the IPG 12 may not reduce a stimulation energy below a minimum effective energy unless a patient is fully aroused (for example, awoken). If a patient is aroused but not fully awoken (for example, at a mild or moderate arousal), then the IPG 12 may reduce the energy to a value equal to or greater than a minimum effective energy. Accordingly, while the stimulation energy may be reduced to avoid fully arousing a patient, the stimulation energy may remain within an effective zone (that is, above or equal to the minimum effective energy) for a longer period of time throughout a patient's sleep.

In some examples, the IPG 12 may determine a degree of arousal based on at least one sleep parameter and classify the degree of arousal into a discrete classification, such as mild arousal, moderate arousal, and full arousal. In various examples, the IPG 12 may determine a degree of arousal, determine how much the degree of arousal deviates from a baseline arousal profile (for example, a baseline movement profile), and classify the deviation into a discrete classification, such as mild arousal, moderate arousal, and full arousal. In some examples, the IPG 12 may determine a degree of arousal, determine how much the degree of arousal deviates from the baseline arousal profile (for example, a baseline movement profile), and evaluate the deviation without classifying the deviation into a discrete classification. However, these non-limiting examples of classifications are provided for purposes of explanation only. In other examples, other classifications (including more or fewer than three classifications) may be implemented.

Stimulation energy titration may include increases and decreases within a titration range that extends from a value equal or greater than the MEE to a maximum treatment energy. The increases and decreases may represent one or more percentage changes (for example, an increase or decrease of 0.1%-100% from a present value) and/or one or more fixed value changes (for example, an increase or decrease by a specific number of mA, for example a change by one or more tenths of a milliamp from a present value or a change of one or more hundredths of a milliamp from a present value). The increases and decreases may depend on a degree of arousal. In some examples, the system may evaluate a fixed step increase or decrease and a percentage increase or decrease and select the larger or smaller of these two values depending on the total amount of change needed and the time over which the change needs to occur. For example, in some cases the stimulation current may change by the larger of 0.1 mA or 5% of a present value.

In at least some of these examples, the IPG 12 may decrease the stimulation energy in response to a detected arousal by a fixed amount or to a fixed value based on the degree of arousal. Using the mild, moderate, and full arousal states as non-limiting examples of degrees of arousal, the IPG 12 may decrease the stimulation current, or another electrical stimulation energy parameter, by a first percentage for a mild arousal, and the IPG 12 may decrease the stimulation current, or another electrical stimulation energy parameter, by a second percentage that is larger than the first percentage for a moderate arousal and the IPG 12 may decrease the stimulation current, or another electrical stimulation energy parameter, by a third percentage that is larger than the first and second percentages for a full arousal. The percentage change may range from 0.1%-100% depending on the degree of arousal.

In another example, the IPG 12 may decrease the stimulation energy by a first step-size for a mild arousal, and the IPG 12 may decrease the stimulation energy by a second step-size that is larger than the first step-size for a moderate arousal and a third step-size for a full arousal.

In another example, the IPG 12 may decrease the stimulation energy in response to a detected arousal to be greater than a minimum effective energy by a certain percentage (for example, 20% greater than, 18% greater than, 15% greater than, or some other value) or by a certain amperage (for example, 3.5 mA, 3 mA, 2.75 mA, 2.5 mA, or some other value) for a mild arousal, and the IPG 12 may decrease the stimulation energy to be greater than a minimum effective energy by a certain percentage (for example, 15% greater than, 10% greater than, 5% greater than, 0% greater than, or some other value) or by a certain value (for example, 2.5 mA, 2 mA, 1 mA, 0.5 mA or some other value for adjustment of stimulation current, or a specific number of volts, seconds, etc. for other electrical stimulation energy parameters) for a moderate arousal.

In various examples, the IPG 12 may not classify the degree of arousal into a discrete classification. In some examples, the IPG 12 may determine a degree of arousal on a continuous scale and respond to the arousal accordingly. In various examples, the IPG 12 may determine a degree of arousal on a continuous scale, determine how much the degree of arousal deviates from a baseline arousal profile (for example, a baseline movement profile), and respond to the arousal accordingly.

For example, the IPG 12 may determine the degree of arousal as a range of angular degrees rolled in a certain amount of time. A greater number of angular degrees rolled in a certain amount of time may correspond to a higher degree of arousal, and thus a greater reduction in stimulation energy. For example, if the patient rolls 40°, the IPG 12 may reduce the stimulation energy by a small amount as compared with, for example, a 1600 roll for which the IPG 12 may reduce the stimulation energy by a larger amount.

In various examples, an amount by which the IPG 12 reduces the stimulation energy in response to a detected arousal is correlated to the degree of arousal of the patient. For purposes of explanation, non-limiting examples are provided in which the degree of arousal of the patient is quantified by a number of angular degrees rolled in a certain period of time. However, other metrics, such as a number of discrete rolls in a certain period of time, may be used in various examples.

In some examples, the IPG 12 reduces the stimulation energy in a linear relationship with the number of angular degrees rolled. For example, for each degree rolled in excess of a baseline amount in a certain period of time, the IPG 12 may reduce the stimulation current by 0.01 mA. Thus, if the patient rolls 100°, the IPG 12 may reduce the stimulation current by 1 mA. In some examples, the IPG 12 reduces the stimulation current in a linear relationship with the number of angular degrees rolled in addition to a fixed value, such as 0.5 mA. Thus, if the patient rolls 100°, the IPG 12 may reduce the stimulation current by 1.5 mA (that is, 1 mA from the patient rolling 100°, plus 0.5 mA as a baseline fixed value). In some examples, the amount of reduction may be a percentage of a present value or a combination of a percentage and a fixed value.

In various examples, the IPG 12 may reduce the stimulation energy in a linear relationship with the number of angular degrees rolled only after the patient has rolled a threshold number of angular degrees. For example, if the threshold number of angular degrees is 25°, the IPG 12 may not reduce the stimulation energy until the patient has rolled at least 25°. In some examples, the IPG 12 may begin counting an amount by which to reduce the stimulation energy after the threshold number of angular degrees. For example, if the threshold number of angular degrees is 25°, the IPG 12 may only reduce the stimulation energy for each degree rolled above 25°. In other examples, the IPG 12 may reduce the stimulation energy for all degrees rolled past the threshold number of angular degrees. For example, if the threshold number of angular degrees is 25°, the IPG 12 may reduce the stimulation energy for all degrees rolled including the initial 25°. The threshold number of angular degrees may be related to a baseline patient movement profile in some examples, or may be unrelated to a baseline patient movement profile in other examples.

In various examples, therefore, the IPG 12 may reduce the stimulation energy in a linear relationship with the degree of arousal. In other examples, the IPG 12 may reduce the stimulation energy in a relationship other than a linear relationship with the degree of arousal. For example, the IPG 12 may reduce the stimulation energy in a logarithmic, exponential, stepwise, or some other relationship with the degree of arousal.

In any of the foregoing examples, the IPG 12 may implement a maximum amount by which the stimulation energy is reduced. For example, the IPG 12 may reduce the stimulation energy to no lower than the minimum effective energy. Accordingly, if any of the examples above would reduce the stimulation energy to below the minimum effective energy, the IPG 12 may instead set the stimulation energy to be equal to the minimum effective energy.

In still other examples, other approaches may be implemented to decrease the stimulation energy based on an increase in arousal. Accordingly, while examples of FIG. 6 are discussed in connection with certain states of arousal (for example, a mild, moderate, and full state of arousal) and certain amounts by which the stimulation energy may be reduced, the disclosure is applicable to a large variety of approaches to reducing stimulation energy to a value equal to or greater than a minimum effective energy based on a degree of arousal.

In some examples, stimulation parameters may vary depending on a present state of the patient. For example, stimulation parameters such as a minimum effective energy and a target energy may vary depending on a sleeping position of the patient. Patients may have less difficulty breathing in some positions than others. Accordingly, less stimulation energy may be required when a patient is in a position for which the patient has less difficulty breathing. These positions may therefore have lower minimum effective energy and target energy levels.

A correlation between sleeping position and sleep disordered breathing intensity may be particularly pronounced for obstructive breathing disorders such as OSA and/or a combination apnea presenting with both OSA and CSA. A tongue position, airway shape, airway stiffness, degree of airway collapse and/or other obstruction parameters may correlate with and be affected by sleeping position. For example, some patients with obstructive breathing disorders may experience substantial difficulty breathing in a supine position, but may experience only slight difficulty breathing in a prone position. In this example, it may be desirable for the IPG 12 to apply more stimulation to a hypoglossal nerve or an ansa cervicalis nerve of the patient in the supine position than in the prone position given that the patient has more difficulty breathing in the supine position.

For purposes of explanation, an example is provided in which different target energy levels and minimum effective energy levels are established for supine, prone, right-side, and left-side positions. In other examples, additional, fewer, or different sleeping positions may be contemplated for establishing target energy levels and minimum effective energy levels. In various examples, target energy levels and minimum effective energy levels may be established for each individual patient. For example, a profile may be established for a patient during a calibration phase with a physician during which the physician determines appropriate values for the target energy and the minimum effective energy.

FIG. 7 illustrates a simplified graph 700 of a stimulation energy according to an example. The graph 700 is simplified by showing the stimulation energy increasing linearly to a specific target energy. In various examples, however, the stimulation energy may be titrated to increase and/or decrease in a non-linear fashion to a target energy, which may change over time.

The graph 700 includes a y-axis 702 and an x-axis 704. The y-axis 702 indicates a magnitude of energy provided by the IPG 12 to at least one of the leads 13. The x-axis 704 indicates an elapsed time for a sleeping period 710. Similarly to the sleeping period 610, the sleeping period 710 is within and may be less than or equal to the daily sleeping period pre-programmed into the IPG 12. The patient being considered to be asleep may be based on the satisfaction of pre-programmed sleep indication conditions as discussed above. The energy trace 799 is illustrated in phantom as a non-limiting example of multiple stimulation energy signals being provided to multiple target nerves in combination. The multiple target nerves may include multiple upper-airway nerves and/or a combination of one or more upper-airway nerves with one or more phrenic nerves. Thus, stimulation energy signals according to two or more of the examples shown in FIGS. 6, 7, 8A, and 9 may be combined to provide multiple energy signals.

The graph 700 includes a sleeping time period 710, a first sub-interval 712, a second sub-interval 714, a third sub-interval 716, and a fourth sub-interval 718. The sub-intervals 712-718 are similar to the sub-intervals 612-616, and are not separated by periods of zero stimulation energy. However, each of the sub-intervals 712-718 corresponds to a respective sleeping position and is thus associated with different stimulation parameters. For example, each of the sub-intervals 712-718 is associated with a respective minimum effective energy and a respective target energy. For purposes of explanation, non-limiting examples are described in which a patient is in one of a supine position, a prone position, and a side position (for example, a right-side position or left-side position).

Notably, unlike FIG. 6 for which the MEE 620 and the target energy 622 remained constant, in FIG. 7, a value of a minimum effective energy and/or a target energy delivered to upper-airway nerves and/or phrenic nerves may depend at least in part on a sleeping position of the patient. For example, patients with sleep apnea may experience more or less difficulty breathing in different sleeping positions, and may therefore benefit from stronger stimulation in certain sleeping positions and weaker stimulation in other sleeping positions. For instance, because tongue position, airway shape, airway stiffness, degree of airway collapse and/or other obstruction parameters may correlate and vary with sleeping position, the amount of stimulation energy required to relieve the obstruction may also correlate and vary with the sleeping position.

As another example, the effectiveness of phrenic nerve stimulation on breathing may depend on upper airway patency and the IPG 12 may be configured to be able to adjust a value of a target energy and/or other stimulation waveform properties for phrenic nerve stimulation based on the sleeping position of the patient and the status of the upper airway. Similarly, the effectiveness of upper-airway nerve stimulation on breathing may depend on diaphragmatic stimulation and/or lung inflation and the IPG 12 may be configured to be able to adjust a value of a target energy and/or other stimulation waveform properties for upper-airway nerve stimulation based on the sleeping position of the patient and the status of the diaphragm and/or lungs. As a further consideration, in the case of transvenous lead electrodes, the position of the electrodes relative to the target nerve may shift with patient position. This shifting may require stimulation energy changes to maintain an effective level of nerve stimulation. Thus, while shown separately for clarity, a therapy according to FIG. 6 (or a more granular version as shown in FIG. 8A) may be combined with a therapy according to FIG. 7 (or a more granular version as shown in FIG. 9) for example to provide a combination apnea therapy targeting both upper-airway nerves and the phrenic nerve.

In the non-limiting example of FIG. 7, the IPG 12 may be configured to be able to deliver stronger stimulation to a target nerve when the patient is in the supine position (for example, during the first sub-interval 712 and the third sub-interval 716), weaker stimulation when the patient is in the side position (for example, during the second sub-interval 714), and even weaker stimulation when the patient is in the prone position (for example, during the fourth sub-interval 718). Different patients may benefit from different stimulation parameters, such as the strongest stimulation to the target nerve(s) being in the prone position and the weakest stimulation to the target nerve(s) being in the supine position. In various examples, a patient profile may be calibrated (for example, by a physician monitoring the patient) to set the stimulation parameters for each sleeping position.

During the first sub-interval 712, the IPG 12 determines a sleeping position of the patient. For example, the IPG 12 may determine the sleeping position of the patient based on patient-position information received from the at least one accelerometer 36. For purposes of example, the IPG 12 may determine that the patient is in a supine position during the first sub-interval 712. The IPG 12 determines a first minimum effective energy (MEE) 720 and a first target energy 722 based on the sleeping position of the patient. For example, the patient may be associated with a sleep profile (which may be determined with assistance from a physician during a calibration phase) indicating a value of the first MEE 720 and the first target energy 722 for the supine position.

Over the first sub-interval 712, the IPG 12 applies a stimulation energy indicated by a first energy trace 724 that ramps up from the first MEE 720 to the first target energy 722. As discussed above, the IPG 12 may ramp up the stimulation energy in a plurality of steps over one or more sub-intervals. Once the stimulation energy reaches the first target energy 722, the IPG 12 maintains the stimulation energy at the first target energy 722. The IPG 12 may maintain the stimulation energy at the first target energy 722 until the patient rolls from the supine position to another position at a first time 726, at which point the first sub-interval 712 ends and the second sub-interval 714 begins.

After the patient rolls to a new position at the first time 726, the IPG 12 determines a sleeping position to which the patient has rolled. For example, the IPG 12 may determine the sleeping position of the patient based on patient-position information received from the at least one accelerometer 36. For purposes of example, the IPG 12 may determine that the patient is in a side position (for example, a right-side position or a left-side position) during the second sub-interval 714. The IPG 12 determines a second minimum effective energy (MEE) 728 and a second target energy 730 based on the sleeping position of the patient. For example, the patient may be associated with a sleep profile (which may be determined with assistance from a physician during a calibration phase) indicating a value of the second MEE 728 and the second target energy 730 for the side position.

Over the second sub-interval 714, the IPG 12 applies a stimulation energy indicated by a second energy trace 732 that ramps up from the second MEE 728 to the second target energy 730. After the patient rolls to the side position at the first time 726, the IPG 12 sets the stimulation energy to the second MEE 728 for the second sub-interval 714. As discussed above, the IPG 12 may ramp up the stimulation energy in a plurality of steps over one or more sub-intervals. Once the stimulation energy reaches the second target energy 730, the IPG 12 maintains the stimulation energy at the second target energy 730. The IPG 12 may maintain the stimulation energy at the second target energy 730 until the patient rolls from the side position to another position at a second time 734, at which point the second sub-interval 714 ends and the third sub-interval 716 begins.

After the patient rolls to a new position at the second time 734, the IPG 12 determines a sleeping position to which the patient has rolled. For example, the IPG 12 may determine the sleeping position of the patient based on patient-position information received from the at least one accelerometer 36. For purposes of example, the IPG 12 may determine that the patient is in the supine position during the third sub-interval 716. The IPG 12 determines a third minimum effective energy (MEE) 736 and a third target energy 738 based on the sleeping position of the patient. For example, the patient may be associated with a sleep profile (which may be determined with assistance from a physician during a calibration phase) indicating a value of the third MEE 736 and the third target energy 738 for the side position.

In some examples, both the first sub-interval 712 and the third sub-interval 716 may correspond to the patient being in a supine position. In various examples, parameters such as a minimum effective energy and a target energy may be the same for the sub-intervals 712, 716, because the parameters may be determined based on the sleeping position of the patient. The first MEE 720 and the third MEE 736 may therefore have equal values, and the first target energy 722 and the third target energy 738 may have equal values.

Over the third sub-interval 716, the IPG 12 applies a stimulation energy indicated by a third energy trace 740 that ramps up from the third MEE 736 to the third target energy 738. After the patient rolls to the supine position at the second time 734, the IPG 12 sets the stimulation energy to the third MEE 736 for the third sub-interval 716. Whereas the stimulation energy may decrease at the first time 726 (for example, because the second MEE 728 for the side position is less than the first target energy 722 for the supine position), the stimulation energy may increase at the second time 734 (for example, because the third MEE 736 is greater than the second target energy 730).

As discussed above, the IPG 12 may ramp up the stimulation energy in a plurality of steps over one or more sub-intervals. Once the stimulation energy reaches the third target energy 738, the IPG 12 maintains the stimulation energy at the third target energy 738. The IPG 12 may maintain the stimulation energy at the third target energy 738 until the patient rolls from the supine position to another position at a third time 742, at which point the third sub-interval 716 ends and the fourth sub-interval 718 begins.

After the patient rolls to a new position at the third time 742, the IPG 12 determines a sleeping position to which the patient has rolled. For example, the IPG 12 may determine the sleeping position of the patient based on patient-position information received from the at least one accelerometer 36. For purposes of example, the IPG 12 may determine that the patient is in a prone position during the fourth sub-interval 718. The IPG 12 determines a fourth minimum effective energy (MEE) 744 and a fourth target energy 746 based on the sleeping position of the patient. For example, the patient may be associated with a sleep profile (which may be determined with assistance from a physician during a calibration phase) indicating a value of the fourth MEE 744 and the fourth target energy 746 for the side position.

Over the fourth sub-interval 718, the IPG 12 applies a stimulation current indicated by a fourth energy trace 748 that ramps up from the fourth MEE 744 to the fourth target energy 746. After the patient rolls to the side position at the third time 742, the IPG 12 sets the stimulation energy to the fourth MEE 744 for the fourth sub-interval 718. As discussed above, the IPG 12 may ramp up the stimulation energy in a plurality of steps over one or more sub-intervals. Once the stimulation energy reaches the fourth target energy 746, the IPG 12 maintains the stimulation energy at the fourth target energy 746.

The IPG 12 may repeat one or more of the stimulation energy patterns shown for sub-intervals 712, 714, 716, and 718 and maintain the stimulation energy at a value greater than or equal to a respective MEE until and unless the IPG 12 detects full arousal of the patient. Upon full arousal, regardless of patient position, the IPG lowers the stimulation energy to zero.

In the foregoing examples of FIGS. 6 and 7, the IPG 12 may ramp up a stimulation energy to a target energy. Although the stimulation-energy traces discussed above may be illustrated as increasingly linearly with a constant slope over various sleeping time periods for purposes of example, the IPG 12 may increase the stimulation energy over a plurality of steps in various examples. Moreover, although a value of a target energy in the graphs 600 and 700 may be depicted as being a fixed value for ease of explanation, in various examples, the target energy may not be a fixed value. As discussed above, the IPG 12 may ramp up the target energy until either a maximum value is met, or the IPG 12 determines that increasing the stimulation energy further may not be beneficial.

The IPG 12 may determine whether increasing the stimulation energy further will be beneficial based on a therapy-effectiveness metric. A therapy-effectiveness metric indicative of how effective the therapy is in addressing a breathing disorder. For example, a therapy-effectiveness metric may include an airway-patency metric indicative of how effectively a patient's airway is stiffened and/or opened in providing upper-airway nerve stimulation. In other examples, the therapy-effectiveness metric may include an entrainment index indicative of whether the patient is entrained to the stimulation energy in providing phrenic nerve stimulation. For purposes of explanation, examples provided below are provided with respect to determining an entrainment index; however, other therapy-effectiveness metrics may be used in addition to, or in lieu of, an entrainment index.

The IPG 12 may determine that increasing the stimulation energy may not be beneficial even if the stimulation energy has not reached a maximum pre-programmed value for stimulation energy. For example, the IPG 12 may determine that the patient is already entrained (for example, by having an entrainment index equal to or above a threshold value) to the electrical stimulation. In such a case, increasing the stimulation energy may be unnecessary or counter-productive by potentially awakening the patient without substantial (or any) improvements to therapy efficacy. In various examples, therefore, a target energy may not be a fixed value, because the IPG 12 increases the stimulation energy until either a maximum pre-programmed value is met or the IPG 12 determines that the stimulation energy does not need to be increased further.

In some examples, the IPG 12 may not only determine that it may not be advantageous to increase the stimulation energy (for example, because the entrainment index is at least a threshold value), but may determine that it may be advantageous to decrease the stimulation energy. The IPG 12 may decrease the stimulation energy over one or more steps responsive to determining that therapy is at or above a threshold level of effectiveness. The steps may be fixed amounts (for example, a number of milliamps) or percentages (for example, a percentage of a present stimulation current). For example, if the present stimulation current is 5 mA and the IPG 12 determines that an entrainment index is above a threshold level, then the IPG 12 may decrease the stimulation current by a 0.1 mA step to 4.9 mA. If the IPG 12 determines that the entrainment index is still above a threshold level, then the IPG 12 may determine that the patient is still entrained. Thus, the target current in this example may decrease by a certain number of milliamps (that is, 0.1 mA) from 5 mA to 4.9 mA.

As another example, if the present stimulation current is 5 mA and the IPG 12 determines that an entrainment index is above a threshold level, then the IPG 12 may decrease the stimulation current by 4% to 4.8 mA. If the IPG 12 determines that the entrainment index is still above a threshold level, then the IPG 12 may determine that the patient is still entrained. Thus, the target current in this example may decrease by a percentage of a present value (that is, 5 mA) from 5 mA to 4.8 mA. The values of 5 mA, 4.9 mA, and 4.8 mA are provided as explanatory examples only and are not limiting of the disclosure; furthermore, while reference is made to a “number of milliamps” above, in other examples a current may be changed based on a current measured on a different order of magnitude, such as microamps. In some cases, the stimulation current may change by the larger of a number of milliamps or a percentage of a present stimulation current, such as the larger of 0.1 mA or 5% of a present value. Therefore, a target energy may not be constant over time, and may refer to an energy at which the patient is entrained and which is lower than a maximum treatment energy value. In some examples, the IPG 12 may continue to reduce the stimulation energy until a minimum effective energy value is reached, or until the patient is no longer entrained, or until some other condition is met.

FIG. 8A illustrates a more detailed graph 800 of a stimulation energy provided by the IPG 12 according to an example. The graph 800 provides more granular details of an example of the stimulation energy. For example, FIG. 8A may provide more granular details of an approach to delivering stimulation energy that is similar to FIG. 6. FIG. 8A is provided with respect to a non-limiting example in which a target nerve is the phrenic nerve. In other examples, similar principles may be applied for other target nerves.

As explained below, the IPG 12 may be configured to be able to monitor at least two parameters while providing the stimulation energy to the patient. A first parameter includes an entrainment index. For example, the IPG 12 may monitor an entrainment index by repeatedly re-calculating the entrainment index while providing the stimulation energy. The IPG 12 may increase the stimulation energy if the entrainment index is below a target value, because the patient's breathing may not be adequately entrained. The IPG 12 may decrease the stimulation energy if the entrainment index is at or above a target value, because the patient may already be entrained and decreasing the stimulation energy may reduce a chance of arousing the patient. As discussed above, the stimulation energy may be gradually reduced provided that the patient is still entrained.

A second parameter includes arousal. As discussed above, the IPG 12 may monitor information received from the at least one accelerometer 36 to determine when a patient is rolling, which may indicate that the patient is aroused. If the IPG 12 determines that the patient is rolling, and may therefore be aroused, the IPG 12 may decrease the stimulation energy. If the IPG 12 determines that the patient is not rolling, and may therefore not be aroused, the IPG 12 may increase or maintain the stimulation energy. In an example, the second parameter may further include patient position as discussed in regard to FIG. 7.

The IPG 12 may be configured to be able to deliver the stimulation energy pursuant to a patient profile for the patient. The patient profile may indicate one or more treatment parameters. The treatment parameters may include various variables characterizing the stimulation energy. The treatment parameters may include a minimum effective energy, a minimum and/or maximum treatment energy to titrate the stimulation energy within, a duration of a current ramping-up period and/or an amount by which to increase the current during the ramping-up period, a duration of a current ramping-down period and/or an amount by which to decrease the current during the ramping-down period, a duration of a time between ramping-up and/or -down periods during which current is maintained, a frequency at which an entrainment index is calculated, a threshold entrainment-index value above which the patient is considered entrained, a threshold entrainment-index value or range to determine when stimulation current should be reduced, maintained, or increased, a pulse amplitude, a number of pulses in a pulse train, a pulse width of the pulses, a time between pulse trains, a waveform of each pulse, a frequency of pulses, a pulse amplitude, a maximum and/or minimum pulse amplitude, a stimulation current, a stimulation voltage, a stimulation polarity (for example, monophasic or biphasic), or any other parameters affecting how the IPG 12 delivers the stimulation energy to the patient.

As discussed with respect to FIG. 8A, the IPG 12 may be configured to be able to deliver stimulation energy while monitoring the patient entrainment and arousal. The IPG 12 may maintain the stimulation energy at a particular electrical stimulation energy for a given period of time, which may be referred to as a hold time or a hold time duration. If the patient is aroused before the given period of time elapses, then the IPG 12 may reduce the stimulation energy. For example, the IPG 12 may ramp down the stimulation energy over a ramping-down period if the IPG 12 determines that the patient is aroused. In some examples, the IPG 12 may also adjust (for example, decrease) treatment parameters such as a minimum and/or maximum value of energy within which the titrate the stimulation energy.

If the patient is not aroused but is also not entrained after the given period of time elapses, then the IPG 12 may increase the stimulation energy. For example, the IPG 12 may increase the stimulation energy over a ramping-up period responsive to determining that the patient is neither aroused nor entrained. In some examples, the IPG 12 may also adjust (for example, increase) a minimum and maximum value of energy within which to titrate the stimulation energy if the patient is still not entrained after some period of time. If the patient is not aroused but is entrained after the given period of time elapses, then the IPG 12 may either maintain or increase the stimulation energy. As noted above, the patient profile may dictate treatment parameters such as the length of the given period of time, an amount by which to increase or decrease the stimulation energy, a rate at which to increase or decrease the stimulation energy, the conditions under which to adjust the minimum and maximum values of energy within which to titrate the stimulation energy, and so forth.

Turning to FIG. 8A, at a first time 802, the IPG 12 begins providing a stimulation energy to the patient upon satisfaction of pre-programmed sleep indication conditions. A first energy trace 803 indicates a value of the stimulation energy. The IPG 12 may determine one or more treatment parameters indicative of the stimulation energy. The IPG 12 may access a patient profile with one or more treatment parameters of the stimulation energy, such as a first minimum treatment energy value (MIN) 804 and a first maximum treatment energy value (MAX) 806. For example, the first MIN 804 may indicate a minimum value of the stimulation energy for the IPG 12 to provide when the patient falls asleep. The first MAX 806 may indicate a maximum value that the stimulation energy may be ramped up to. The first MIN 804 may be equal to or greater than a minimum effective energy (MEE) 808. The MEE 808 is a minimum value for a stimulation energy to modulate a patient's diaphragm and/or upper airway patency.

At the first time 802, the IPG 12 begins ramping up the energy in a step-wise manner until a second time 810. The time between the first time 802 and the second time 810 may be referred to as a first ramping-up period or a first energy step, because the stimulation energy is ramped up between the times 802, 810. The patient profile may indicate treatment parameters such as an amount by which the stimulation energy is ramped up over each ramping-up period, a time over which the stimulation energy is ramped up over each ramping-up period, a time between ramping-up periods, and so forth.

After the first ramping up period ends at the second time 810, the IPG 12 may maintain the value of the stimulation energy for a given period of time indicated by the patient profile. For example, the given period of time may be approximately five minutes; for ease of explanation, the following description assumes that the given period of time is approximately five minutes, although other times may be used in other examples. During this five-minute window (or a window of time less than or greater than five minutes), the IPG 12 may monitor an entrainment index for the patient to determine whether the patient is sufficiently entrained to maintain the stimulation energy at a present value. Determining whether the patient is entrained may include the IPG 12 determining an entrainment index and comparing the entrainment index to a threshold value corresponding to the patient being entrained.

The IPG 12 may also monitor patient arousal (for example, based on patient-position information) to determine whether the patient is aroused, which may warrant decreasing the stimulation energy. If the patient is still not aroused or entrained by the end of the five-minute window, the IPG 12 may increase the stimulation energy.

At a third time 812, the IPG 12 begins a second ramping-up period during which the IPG 12 increases the stimulation energy. The IPG 12 may begin the second ramping-up period because the patient was neither entrained nor aroused by the end of the five-minute window between the second time 810 and the third time 812. The IPG 12 may begin the second ramping-up period at least in part because the patient is not entrained and is not aroused. The IPG 12 ramps up the stimulation energy from the third time 812 to a fourth time 814, at which point the stimulation energy is at a first intermediate energy (IME) 816, which is greater than the first MIN 804 and less than the first MAX 806.

After the second ramping-up period ends at the fourth time 814, the IPG 12 may monitor the entrainment index of the patient over another five-minute window. The IPG 12 may also monitor patient arousal (for example, based on patient-position information) to determine whether the patient is aroused, which may warrant decreasing the stimulation energy. Before the end of the five-minute window, the IPG 12 may determine that the entrainment index meets or exceeds an entrainment threshold indicating that the patient is entrained. Accordingly, although the stimulation energy may be less than the first MAX 806, the IPG 12 may maintain the stimulation energy at the first IME 816 because the patient is entrained. The first IME 816 is thus considered the target energy because the patient is entrained at the first IME 816.

At a fifth time 818, the IPG 12 may determine that the patient has rolled from one position to another based on information received from the at least one accelerometer 36. As discussed above, the patient rolling may indicate that the patient is aroused. It may be advantageous to decrease the stimulation energy responsive to the patient being aroused, even before the five-minute window elapses. The treatment parameters of the patient profile may indicate an amount by which to decrease a minimum and maximum treatment energy responsive to a patient being aroused. Accordingly, at the fifth time 818, the IPG 12 reduces the first MIN 804 to a second minimum treatment energy (MIN) 820 and reduces the first MAX 806 to a second maximum treatment energy (MAX) 822. The second MIN 820 is still greater than the MEE 808.

At the fifth time 818, the IPG 12 reduces the stimulation energy to a value between the second MIN 820 and the second MAX 822. The patient profile may indicate an energy value between the second MIN 820 and the second MAX 822 to set the stimulation energy to. The IPG 12 maintains the stimulation energy at a present value for a period of time indicated by the patient profile. During this period of time, the IPG 12 may monitor an entrainment index to determine if the patient is entrained. The IPG 12 may also monitor patient arousal (for example, based on patient-position information) to determine whether the patient is aroused, which may warrant decreasing the stimulation energy. If the IPG 12 determines that the patient is not entrained nor aroused by the end of the period of time at a sixth time 824, then the IPG 12 may increase the stimulation energy.

At the sixth time 824, the IPG 12 ramps up the stimulation energy over a third ramping-up period to a second intermediate energy (IME) 826 at a seventh time 828. The IPG 12 begins monitoring an entrainment index at the seventh time 828, and may determine that the patient is entrained (for example, because the entrainment index exceeds a threshold value). The second IME 826 is considered a target energy because, even though the second IME 826 is less than the second MAX 822, the IPG 12 has determined that the patient is entrained at the second IME 826. After the IPG 12 completes ramping up the stimulation energy over the third ramping-up period at the seventh time 828, the IPG 12 maintains the stimulation energy at the second IME 826 while monitoring the entrainment index and patient arousal. At an eighth time 830, the IPG 12 may determine that the patient is entrained and not aroused. Accordingly, the IPG 12 may maintain the stimulation energy at the second IME 826.

At a ninth time 832, the IPG 12 may determine that the patient is still entrained and still not aroused, and may reduce the stimulation energy. It may be advantageous to reduce the stimulation energy because a likelihood of arousing the patient is reduced while the patient ideally remains entrained. That is, while the second IME 826 may be effective in entraining the patient, it may be possible to entrain the patient at an even lower energy, which both reduces a chance of awakening the patient and reduces energy consumption by the IPG 12. In some examples, the IPG 12 may reduce the stimulation energy rather than maintaining the stimulation energy if, for example, the patient has been entrained (for example, continuously entrained) for at least a threshold period of time. In another example, the IPG 12 may reduce the stimulation energy rather than maintaining the stimulation energy if, for example, the IPG 12 determines that the entrainment index falls within a certain range of values.

For example, suppose that the IPG 12 determines that the entrainment index is 85%. The IPG 12 may implement multiple ranges or thresholds to determine how to respond to the entrainment index. For example, if the entrainment index is below 70%, the IPG 12 may determine that the patient is not entrained and thus increase the stimulation energy (unless the patient is aroused). If the entrainment index is between 70% and 80%, the IPG 12 may determine that the patient is entrained and thus maintain the stimulation energy. If the entrainment index is above 80%, then the IPG 12 may determine that the patient is entrained but may reduce the stimulation energy. Because the patient may be considered entrained at an entrainment index above 70%, and because indexes above 80% are well above the entrainment threshold, it may be possible and desirable to lower the stimulation energy without compromising entrainment. Accordingly, for an entrainment index of 85% (which is above 80%), the IPG 12 may reduce the stimulation energy.

In various examples, the IPG 12 may be configured to be able to implement a combination of time thresholds and entrainment-index thresholds to determine whether to decrease or maintain the stimulation energy. In still other examples, the IPG 12 may consider additional or different factors when determining whether to decrease or maintain the stimulation energy.

Returning to FIG. 8A, at the ninth time 832, the IPG 12 reduces the stimulation energy. The IPG 12 ramps down the stimulation energy to a value greater than the second MIN 820. The IPG 12 therefore continues to apply stimulation energy between the second MIN 820 and the second MAX 822 (that is, within the effective zone) while reducing the stimulation energy (and thus reducing a likelihood of arousing the patient). The patient profile may indicate an amount by which to reduce the energy over the ramping-down period. The IPG 12 reduces the stimulation energy from the ninth time 832 to a tenth time 834.

At the tenth time 834, the IPG 12 maintains the stimulation energy at a third intermediate energy (IME) 836. The IPG 12 may continue to monitor patient entrainment and/or arousal. Until an eleventh time 838, the IPG 12 may determine that the patient remains entrained at the third IME 836 and is not aroused. At the eleventh time 838, the IPG 12 may determine that a range of energy values within which to titrate the stimulation energy should be increased. For example, the IPG 12 may increase the titration window if the patient has remained un-aroused for at least a threshold period of time, that is, that the patient has experienced uninterrupted (that is, not aroused) sleep for at least a threshold period of time. In some examples, the IPG 12 may increase the minimum and/or maximum treatment energy values throughout the patient's sleep.

Accordingly, at the eleventh time 838, the IPG 12 establishes a third maximum treatment energy value (MAX) 840 and a third minimum treatment energy value (MIN) 842. The IPG 12 begins ramping up the stimulation energy to a value between the third MAX 840 and third MIN 842 from the eleventh time 838 to a twelfth time 844. For example, the patient profile may establish a value of the third MAX 840, the third MIN 842, and the value for the stimulation energy to ramp up to at the twelfth time 844.

After the twelfth time 844, the IPG 12 may monitor an entrainment index and arousal of the patient while maintaining the stimulation energy. At a thirteenth time 846, the IPG 12 may determine that the patient is fully aroused 596. For example, the IPG 12 may determine that the patient has awoken. The IPG 12 stops providing the stimulation energy, and the value of the stimulation energy therefore drops to zero.

Accordingly, between the first time 802 and the thirteenth time 846, the IPG 12 titrates the stimulation energy by increasing and decreasing the stimulation energy within a range of maximum and minimum treatment energy values. The maximum and minimum treatment energy values are equal to or greater than the MEE 808. Accordingly, while the IPG 12 may decrease the stimulation energy responsive to patient arousal, the IPG 12 may continuously provide minimally effective energy (that is, a stimulation energy equal to or greater than the MEE 808) until the patient is fully aroused. In other words, the IPG 12 may continuously apply stimulation energy without any periods during which no energy is provided until the patient is fully aroused.

After the patient is fully aroused at the thirteenth time 846, the patient may eventually go back to sleep. The IPG 12 determines that the patient is no longer fully aroused (but may be mildly or moderately aroused) at the fourteen time 848, and resumes providing a stimulation energy as indicated by the first energy trace 803. The IPG 12 may establish a fourth minimum treatment energy (MIN) 850 and a fourth maximum treatment energy (MAX) 852. The IPG 12 may set the stimulation energy to the fourth MIN 850, and begins monitoring patient arousal and entrainment.

At a fifteenth time 854, the IPG 12 may determine that the patient is not entrained nor aroused. Accordingly, the IPG 12 may increase the stimulation energy. At a sixteenth time 856, however, the IPG 12 may determine that the patient is aroused. For example, the IPG 12 may determine that the patient has rolled, and that the stimulation energy should be reduced.

The IPG 12 may take at least one of several actions in response to patient arousal. For example, at the fifth time 818, the IPG 12 reduced the first MIN 804 to the second minimum treatment energy (MIN) 820 and reduced the first MAX 806 to the second maximum treatment energy (MAX) 822 in response to patient arousal. In other examples, the IPG 12 may reduce the stimulation energy in response to patient arousal without modifying the minimum and maximum values within which the stimulation energy is titrated. For example, the IPG 12 may reduce the stimulation energy without modifying the minimum and maximum treatment energy values if the patient is mildly aroused (for example, by rolling one positional quadrant) and may reduce the stimulation energy as well as the minimum and maximum treatment energy values if the patient is moderately aroused (for example, by rolling two or more positional quadrants within a threshold period of time).

At the sixteenth time 856, the IPG 12 reduces the stimulation energy back to the fourth MIN 850 without modifying the value of the fourth MIN 850 or the fourth MAX 852. For example, the IPG 12 may determine that the patient is only mildly aroused at the sixteenth time 856. The IPG 12 maintains the stimulation energy at the fourth MIN 850 while monitoring patient arousal and entrainment.

At a seventeenth time 858, the IPG 12 determines that the patient is not aroused or entrained, and therefore increases the stimulation energy to the fourth MAX 852. The fourth MAX 852 is therefore considered a target energy regardless of whether or not the patient is entrained, because the stimulation energy is already titrated to a highest permissible value. The IPG 12 may repeat one or more of the stimulation energy patterns shown in the graph 800 and maintain the stimulation energy at a value greater than or equal to a respective MEE until and unless the IPG 12 detects full arousal of the patient. Upon full arousal, the IPG lowers the stimulation energy to zero.

In summary of the example of FIG. 8A, the IPG 12 may deliver stimulation energy while monitoring the patient entrainment and arousal. The IPG 12 may maintain the stimulation energy at a given value for a given period of time, such as five minutes. If the patient is aroused before the given period of time elapses, then the IPG 12 may reduce the stimulation energy to avoid over-stimulating the patient. If the patient is not aroused after the given period of time elapses, and the patient is still not entrained, then the IPG 12 may increase the stimulation energy. If the patient is not aroused after the given period of time elapses but is entrained, then the IPG 12 may maintain or reduce the stimulation energy. For example, the IPG 12 may reduce the stimulation energy if the patient has been entrained for a threshold period of time, and/or if the patient has been asleep for a threshold period of time, and/or if the entrainment index of the patient is sufficiently high, and/or if other conditions are met.

In some examples, the IPG 12 may also adjust the minimum and maximum treatment energy values within which to titrate the stimulation energy. In some examples, the IPG 12 may increase the minimum and maximum treatment energy values after some period of time. For example, the IPG 12 may increase the minimum and maximum treatment energy values over the course of a night's sleep, or over the course of a week of therapy, or over the course of a month of therapy, or some other period of time.

In various examples, the IPG 12 may reduce the minimum and maximum treatment energy values. For example, the IPG 12 may reduce the minimum and maximum treatment energy values if the patient is aroused by a threshold amount. The threshold amount of arousal may include the patient rolling a certain number of times within a given period of time. In some examples, the IPG 12 may reduce the minimum and maximum treatment energy values if the patient is entrained in certain conditions. For example, if the IPG 12 is providing stimulation energy at the minimum value and the patient is still entrained, it may be advantageous to further reduce the minimum value to accommodate even lower stimulation energies without jeopardizing entrainment. In various examples, the IPG 12 may adjust the minimum and maximum treatment energy values to various values provided that the values are greater than a minimum effective energy.

The IPG 12 may therefore adjust treatment parameters dictating a stimulation energy based at least on patient arousal and based on an indication of therapy effectiveness, such as entrainment or airway patency. For example, the IPG 12 may deliver phrenic nerve stimulation and adjust phrenic nerve stimulation parameters based on patient arousal and entrainment. In other examples, the IPG 12 may deliver therapy and adjust treatment parameters based on patient arousal and/or position, but not based on an indication of therapy effectiveness. For example, the IPG 12 may deliver upper-airway nerve stimulation and adjust upper-airway nerve stimulation parameters based on patient arousal and position, but not based on entrainment.

In some examples, the IPG 12 may deliver phrenic nerve stimulation and adjust phrenic nerve stimulation parameters based on patient arousal and entrainment, and the IPG 12 may deliver upper-airway nerve stimulation and adjust upper-airway nerve stimulation parameters based on patient arousal. Accordingly, the IPG 12 may provide at least two different stimulation energies to provide at least two different therapies in combination.

FIG. 8B illustrates stimulation energy titration with variations in minimum and maximum treatment energies according to an example. As shown in FIG. 8B, across a therapy titration sequence, the range between the minimum treatment energies (870a, 870b, 870c, 870d) and the maximum treatment energies (880a, 880b, 880c, and 880d) may vary. For example, the ranges 890a, 890b, 890c, and 890d are not all equal. In some examples, one or more ranges across a therapy sequence may all be the same, may all be different, or may have variations with some ranges being equivalent to others and other ranges being different from one or more other ranges.

As discussed herein, the minimum and maximum treatment energies may vary based on the target nerve, the degree of arousal, the position of the patient, entrainment, and so forth, and one or more of these factors may vary over the course of therapy delivery thus producing different ranges.

FIG. 9 illustrates a graph 900 of a stimulation energy provided by the IPG 12 according to an example. FIG. 9 may provide more granular details of an approach to delivering stimulation energy that is similar to FIG. 7. In at least one example, the graph 900 may provide an example of the IPG 12 providing a stimulation energy to one or more target nerves. The one or more target nerves may include multiple upper-airway nerves, multiple phrenic nerves, and/or a combination of one or more upper-airway nerves with one or more phrenic nerves.

As explained below, the IPG 12 may monitor patient arousal and position. For example, the IPG 12 may monitor information received from the at least one accelerometer 36 to determine whether a patient is rolling and/or what position the patient is in (such as a left-side position, right-side position, supine position, or prone position). A position of the patient may dictate certain treatment parameters, such as a minimum and maximum treatment energy value. Arousal of the patient may dictate how the stimulation energy is titrated between the minimum and maximum treatment energy values. If the IPG 12 determines that the patient is rolling, and may therefore be aroused, the IPG 12 may decrease the stimulation energy. If the IPG 12 determines that the patient is not rolling, and may therefore not be aroused, the IPG 12 may increase or maintain the stimulation energy.

The IPG 12 may deliver the stimulation energy pursuant to a patient profile for the patient indicating one or more treatment parameters. The treatment parameters may include a minimum effective energy, a minimum and maximum treatment energy value to titrate the stimulation energy within, a duration of an energy ramping-up period and/or an amount by which to increase the energy during the ramping-up period, a duration of an energy ramping-down period and/or an amount by which to decrease the energy during the ramping-down period, a duration of a time between ramping-up and/or -down periods during which energy is maintained, a pulse amplitude, a number of pulses in a pulse train, a pulse width of the pulses, a time between pulse trains, a waveform of each pulse, a frequency of pulses, a pulse amplitude, a maximum pulse amplitude, a stimulation current, a stimulation voltage, a stimulation polarity (for example, monophasic or biphasic), stimulation energy ramps, or any other parameters affecting how the IPG 12 delivers the stimulation energy to the patient.

As discussed with respect to FIG. 9, the IPG 12 may deliver stimulation energy while monitoring the patient position and arousal. The IPG 12 may maintain the stimulation energy at a given value for a given period of time. If the patient is aroused before the given period of time elapses, then the IPG 12 may reduce the stimulation energy. For example, the IPG 12 may ramp down the stimulation energy over a ramping-down period. If the patient is not aroused after the given period of time elapses, then the IPG 12 may increase the stimulation energy. For example, the IPG 12 may increase the stimulation energy over a ramping-up period.

The IPG 12 may titrate the stimulation energy up and/or down between a minimum and maximum value. The patient profile may dictate a minimum and maximum treatment energy value for each sleeping position. In some examples, the IPG 12 may adjust the minimum and maximum treatment energy values based on a certain amount of time elapsing. For example, the IPG 12 may increase the minimum and/or maximum treatment energy values over the course of a night, or a week, or a month, or some other period of time. As noted above, the patient profile may dictate treatment parameters such as the length of the given period of time, an amount by which to increase or decrease the stimulation energy, a rate at which to increase or decrease the stimulation energy, the conditions under which to adjust the minimum and maximum values of energy within which to titrate the stimulation energy, and so forth.

Turning to FIG. 9, an example is provided in which the IPG 12 delivers phrenic nerve stimulation and upper-airway nerve stimulation in combination. The graph 900 includes the first energy trace 803 indicating a stimulation energy provided to a first nerve and a second energy trace 902 indicating a stimulation energy provided to a second nerve. The first nerve may be the phrenic nerve and the second nerve may be the upper-airway nerve or vice versa.

At a first time 904, the IPG 12 determines that the patient is asleep in a first sleeping position. The patient being considered to be asleep may be based on the satisfaction of pre-programmed sleep indication conditions as discussed above. For example, the first sleeping position may be a supine position. The IPG 12 may access a patient profile to determine a first minimum treatment energy value (MIN) 906 and a first maximum treatment energy value (MAX) 908 to apply while the patient is in the supine position. The IPG 12 begins applying the stimulation energy at the first MIN 906 and begins ramping up over a first energy step to a first intermediate energy (IME) 910. The first MIN 906 and the first MAX 908 may be applicable to the second energy trace 902, but not the first energy trace 803; accordingly, the first energy trace 803 may not stay within the bounds of the MIN and MAX values illustrated in FIG. 9. The first energy trace 803 is illustrated in phantom as a non-limiting example of multiple stimulation energies being provided to multiple target nerves in combination, and is not necessarily limiting on the second energy trace 902.

At a second time 912, the IPG 12 finishes ramping up the stimulation energy to the first IME 910. The IPG 12 maintains the stimulation energy at the first IME 910 for a period of time, such as five minutes. During this period of time, the IPG 12 monitors patient arousal and position based on information received from the at least one accelerometer 36. By the end of the period of time at a third time 914, the IPG 12 determines that the patient has not been aroused and is in the same sleeping position. Accordingly, the IPG 12 ramps up the energy at the third time 914. The patient profile may specify parameters such as a time over which to ramp up the energy, an amount by which to ramp up the energy, a period of time to wait before determining whether to ramp up the energy, and so forth.

At the third time 914, the IPG 12 ramps up the stimulation energy until a fourth time 916 over a second energy step. At the fourth time 916, the stimulation energy reaches a second intermediate energy (IME) 918. The IPG 12 maintains the stimulation energy at the second IME 918. The second energy step from the first IME 910 to the second IME 918 may be of an equal magnitude to the first energy step from the first MIN 906 to the first IME 910 in some examples. In other examples, the energy-step magnitudes may be different. For example, the energy-step magnitudes may incrementally decrease as the stimulation energy approaches the first MAX 908. In another example, the energy-step magnitudes may incrementally increase as the stimulation energy approaches the first MAX 908.

This process of ramping up the energy and waiting for a period of time to again ramp up the energy if the patient is not aroused continues until a fifth time 920, at which point the IPG 12 determines that the patient has rolled from the first sleeping position to a second sleeping position. For example, the second sleeping position may be a side position.

At the fifth time 920, the IPG 12 determines based on the patient profile a second minimum treatment energy (MIN) 922 and a second maximum treatment energy (MAX) 924 corresponding to the second sleeping position. The second MIN 922 and the second MAX 924 may be lower than the first MIN 906 and the first MAX 908. For example, the patient may have less difficulty sleeping in the second sleeping position than the first sleeping position. Less stimulation energy may therefore be warranted in the second sleeping position.

Other than the different minimum and maximum treatment energy values, the IPG 12 may deliver therapy in substantially the same manner in the second sleeping position as the first sleeping position. The IPG 12 may therefore continue to ramp up the energy and wait for a period of time to again ramp up the energy if the patient is not aroused until a sixth time 926. At the sixth time 926, the IPG 12 determines that the patient is fully aroused 596 (for example, awake) and stops providing stimulation energy.

At a seventh time 928, the IPG 12 determines that the patient is no longer fully aroused (but may be mildly or moderately aroused). However, the patient is asleep in a third sleeping position, such as a prone position. The IPG 12 determines based on the patient profile a third minimum treatment energy (MIN) 930 and a third maximum treatment energy (MAX) 932 corresponding to the third sleeping position. The third MIN 930 may be lower than the first MIN 906 and the second MIN 922, and the third MAX 932 may be lower than the first MAX 908 and the second MAX 924. For example, the patient may have less difficulty sleeping in the third sleeping position than in either the first sleeping position or the second sleeping position.

Other than the different minimum and maximum treatment energy values, the IPG 12 may deliver therapy in substantially the same manner in the third sleeping position as the first and second sleeping positions. The IPG 12 may therefore continue to ramp up the energy and wait for a period of time to again ramp up the energy if the patient is not aroused until an eighth time 934. At the eighth time 934, the IPG 12 determines that the patient is aroused. For example, the IPG 12 may determine that the patient has rolled from one positional quadrant to another. The IPG 12 may reduce the stimulation energy to the third MIN 930 responsive to determining that the patient is aroused.

However, the IPG 12 may determine that once the patient stops rolling, the patient has ultimately returned to the third sleeping position. For example, the patient may roll from the prone position to the side position, and then immediately back to the prone position. Accordingly, while the IPG 12 reduces the stimulation energy to the third MIN 930 responsive to the patient being aroused, the IPG 12 continues to implement the third MIN 930 and the third MAX 932 responsive to the patient returning to the third sleeping position.

The IPG 12 may therefore continue to ramp up the energy from the third MIN 930 and wait for a period of time to again ramp up the energy if the patient is not aroused. The IPG 12 may repeat one or more of the stimulation energy patterns shown in the graph 800 and maintain the stimulation energy at a value greater than or equal to a respective MEE until and unless the IPG 12 detects full arousal of the patient. Upon full arousal, the IPG lowers the stimulation energy to zero.

Accordingly, the IPG 12 may titrate stimulation energy based on several different approaches. In a first approach, the IPG 12 may titrate the stimulation energy based on patient arousal and/or a metric indicative of therapy effectiveness, such as an entrainment index. In a second approach, the IPG 12 may not consider a metric such as an entrainment index, but may titrate the stimulation energy based on patient arousal and/or position. The IPG 12 determines a minimum and maximum stimulation-energy value based on patient position and ramps the stimulation energy up between the minimum and maximum values until either the patient is aroused (in which case the IPG 12 reduces the stimulation energy) or the stimulation energy reaches the maximum value (in which case the IPG 12 maintains the stimulation energy at the maximum value).

In either approach, the IPG 12 may deliver therapy pursuant to treatment parameters associated with a patient profile. The IPG 12 may deliver stimulation energy to multiple nerves with different approaches in combination, such as by delivering stimulation energy pursuant to the first approach to a phrenic nerve in coordination with delivering stimulation energy pursuant to the second approach to an airway nerve (for example, a hypoglossal nerve and/or an ansa cervicalis nerve).

A patient profile may be established during a calibration phase before therapy is delivered during a therapy-delivery stage. During the calibration phase, a physician may monitor a patient to establish various treatment parameters, such as minimum and maximum treatment energy values. The physician may also establish baseline parameters, such as a baseline patient movement profile indicating a baseline amount that the patient moves throughout the night without any stimulation. This patient movement profile may be used to determine if a patient is aroused by the stimulation energy, because deviations from the baseline patient movement profile may be attributed to the stimulation energy.

After the calibration phase, the IPG 12 may begin applying stimulation to the patient during a therapy-delivery stage. During the therapy-delivery stage, the patient profile may optionally be updated. For example, a physician may monitor how a patient is responding to therapy and may adjust the patient profile based thereon.

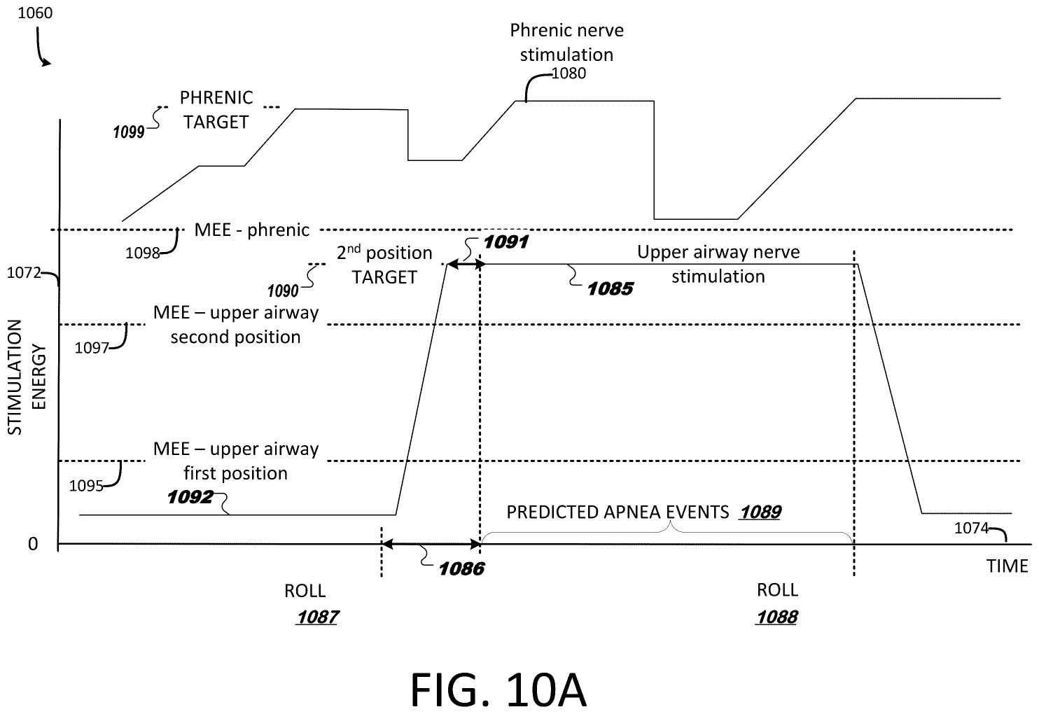

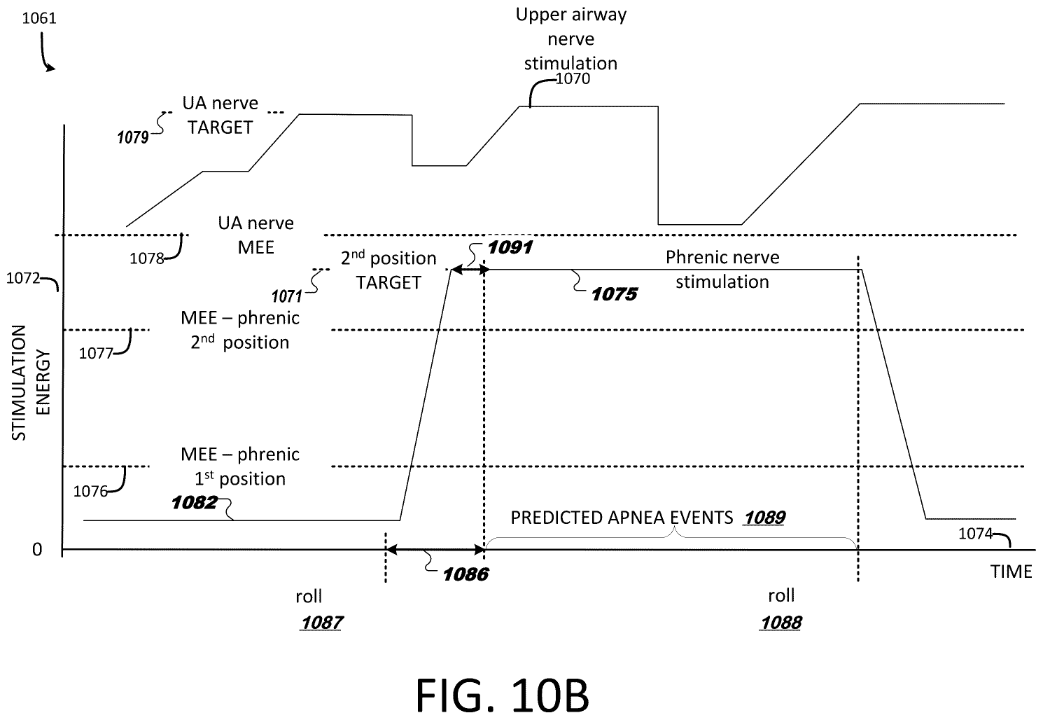

FIG. 10A illustrates a simplified graph of a stimulation energy titrated according to predicted patient apnea responses, also referred to as predicted patient responses, according to an example. The graph 1060 is simplified by showing the stimulation energy increasing and decreasing linearly. In various examples, however, the stimulation energy may be titrated to increase and/or decrease in a non-linear fashion to a target energy, which may change over time. The graph 1060 includes a y-axis 1072 and an x-axis 1074. The y-axis 1072 indicates a magnitude of energy provided by the IPG 12 to at least one phrenic nerve stimulation lead 14 and/or at least one upper-airway nerve stimulation lead 16. Although both of the phrenic and upper-airway nerve stimulation traces are shown in FIG. 10A, this is an example only and the IPG 12 may provide either one of these with or without the other. The x-axis 1074 indicates an elapsed time during a sleeping period of a patient. The patient being considered to be asleep is based on the satisfaction of pre-programmed sleep indication conditions as discussed above.

The phrenic nerve stimulation trace 1080 illustrates an example of titration by the IPG 12 between an MEE 1098 for the phrenic nerve and a target 1099 for the phrenic nerve. The titration of the phrenic nerve stimulation as illustrated in FIG. 10A may be as described in one or more of the examples shown in FIGS. 6-9.

The upper-airway nerve stimulation trace 1085 illustrates an example of titration by the IPG 12 based on a predicted sleep apnea response, also referred to as a predicted patient response, of a particular patient to a change in a degree of arousal based on sleeping position. In this example illustration, a base line analysis of a sleep apnea patient, in the absence of electrical stimulation entirely or in the absence of upper airway stimulation or phrenic nerve stimulation, may show that the patient does not experience sleep apnea events in a first sleep position but that sleep apnea events begin after an approximate time interval 1086 in a second sleep position of the patient. For example, the first sleep position may be a prone, left side, right side, or supine position and the second sleep position may also be one of prone, left side, right side, or supine but different from the first sleep position. Thus, a roll 1087 to the second position may be predictive of the onset of sleep apnea events 1089.

Based on this predictive knowledge, the IPG 12 may maintain the electrical stimulation energy to the upper-airway nerve at zero or at a low level (for example, as illustrated by the trace portion 1092 below any MEE for upper airway, including MEE 1095 for a first position and MEE 1097 for a second position) to preserve battery life and minimize the probability of disturbing the patient. However, when and if the IPG 12 detects the roll 1087 to the second position, the IPG 12 may increase the stimulation energy to the upper-airway nerve above the MEE 1092 for the first position and reach a target energy 1090 for the second position prior to the onset of the predicted sleep apnea events 1089. The IPG 12 may reach the target energy 1090 at a time interval 1091 prior to the onset of the predicted sleep apnea events 1089.

The IPG 12 may maintain the stimulation energy at the target and/or titrate based on detected arousals as described for example in regard to FIGS. 6-9 until the IPG 12 detects a roll 1088 to the first position or a third position different from the first or second. At this point the IPG 12 may reduce the stimulation energy to zero or to a minimal but non-zero level. The minimal but non-zero level may enable a reduction in the ramp time to a target energy while still preserving battery life and avoiding patient disruption. Data collection for a patient over time by the IPG 12 and/or the external computing device 210 may enable a machine learning analysis with which to refine and optimize the predictive performance of the IPG 12 for both the onset of sleep apnea and the various MEE and target stimulation energies needed to treat the sleep apnea of a particular patient. Although FIG. 10A illustrates an increase in MEE from the first position to the second position, this is an example only and a decrease in MEE or a lack of change in the MEE between two positions is within the scope of the disclosure.

FIG. 10B illustrates a simplified graph of a stimulation energy titrated according to predicted patient apnea responses, also referred to as predicted patient responses, according to an example. The graph 1061 is simplified by showing the stimulation energy increasing and decreasing linearly. In various examples, however, the stimulation energy may be titrated to increase and/or decrease in a non-linear fashion to a target energy, which may change over time. Similarly to FIG. 10A, the graph 1061 includes a y-axis 1072 and an x-axis 1074. The y-axis 1072 indicates a magnitude of energy provided by the IPG 12 to at least one phrenic nerve stimulation lead 14 and/or at least one upper-airway nerve stimulation lead 16. Although both of the phrenic and upper-airway nerve stimulation traces are shown in FIG. 10B, this is an example only and the IPG 12 may provide either one of these with or without the other. The x-axis 1074 indicates an elapsed time during a sleeping period of a patient. The patient being considered to be asleep is based on the satisfaction of pre-programmed sleep indication conditions as discussed above.

The upper-airway nerve stimulation trace 1070 illustrates an example of titration by the IPG 12 between a MEE 1078 for the upper-airway nerve and a target 1079 for the upper-airway nerve. The titration of the upper-airway nerve stimulation as illustrated in FIG. 10B may be as described in one or more of the examples shown in FIGS. 6-9.

The phrenic nerve stimulation trace 1075 illustrates an example of titration by the IPG 12 based on a predicted sleep apnea response, also referred to as a predicted patient response of a particular patient to a change in a degree of arousal based on sleeping position. In this example illustration, a base line analysis of a sleep apnea patient, in the absence of electrical stimulation entirely or in the absence of one of upper airway stimulation or phrenic nerve stimulation, may show that the patient does not experience sleep apnea events in a first sleep position but that sleep apnea events begin after an approximate time interval 1086 in a second sleep position of the patient. For example, the first sleep position may be a prone, left side, right side, or supine position and the second sleep position may also be one of prone, left side, right side, or supine but different from the first sleep position. Thus, a roll 1087 to the second position may be predictive of the onset of sleep apnea events 1089.

Based on this predictive knowledge, the IPG 12 may maintain the electrical stimulation energy to the phrenic nerve at zero or at a low level (for example, as illustrated by the trace portion 1082 below any MEE for the phrenic nerve, including MEE 1076 for a first position and MEE 1077 for a second position) to preserve battery life and minimize the probability of disturbing the patient. However, when and if the IPG 12 detects the roll 1087 to the second position, the IPG 12 may increase the stimulation energy to the phrenic nerve above the MEE 1076 for the first position and reach a target energy 1071 for the second position prior to the onset of the predicted sleep apnea events 1089. The IPG 12 may reach the target energy 1071 at a time interval 1091 prior to the onset of the predicted sleep apnea events 1089. The IPG 12 may maintain the stimulation energy at the target and/or titrate based on detected arousals as described for example in regard to FIGS. 6-9 until the IPG 12 detects a roll 1088 to the first position or a third position different from the first or second.

At this point, the IPG 12 may reduce the stimulation energy to zero or to a minimal but non-zero level. The minimal but non-zero level may enable a reduction in the ramp time to a target energy while still preserving battery life and avoiding patient disruption. Data collection for a patient over time by the IPG 12 and/or the external computing device 210 may enable a machine learning analysis with which to refine and optimize the predictive performance of the IPG 12 for both the onset of sleep apnea and the various MEE and target stimulation energies needed to treat the sleep apnea of a particular patient. Although FIG. 10B illustrates an increase in MEE from the first position to the second position, this is an example only and a decrease in MEE or a lack of change in the MEE between two positions is within the scope of the disclosure.

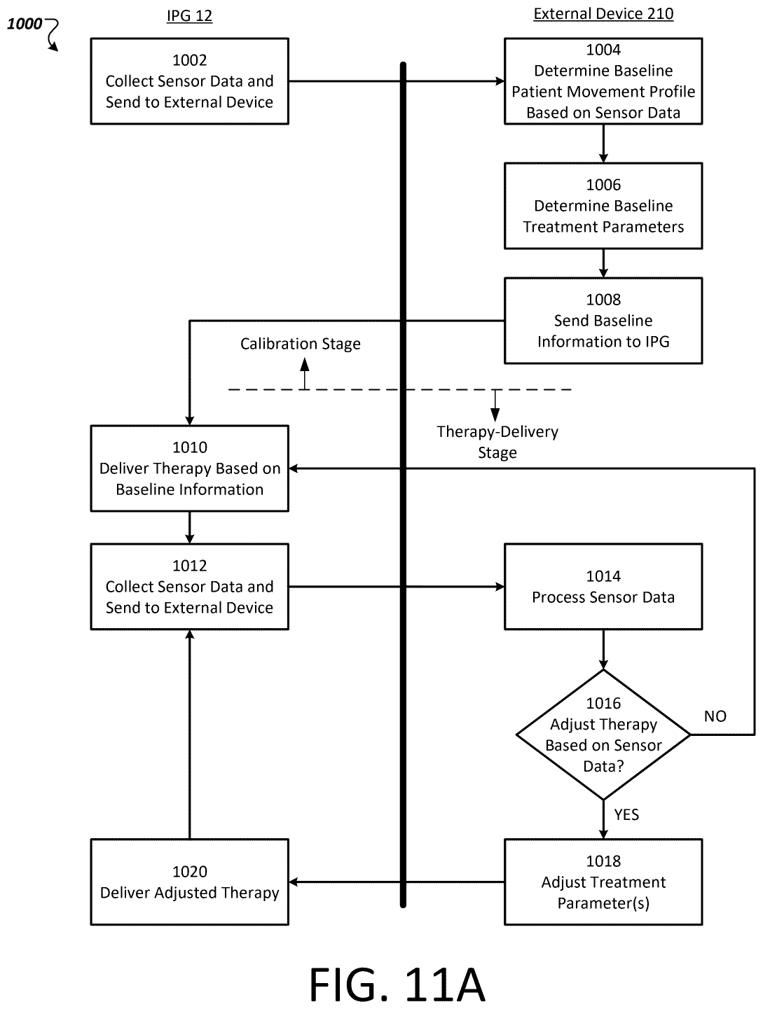

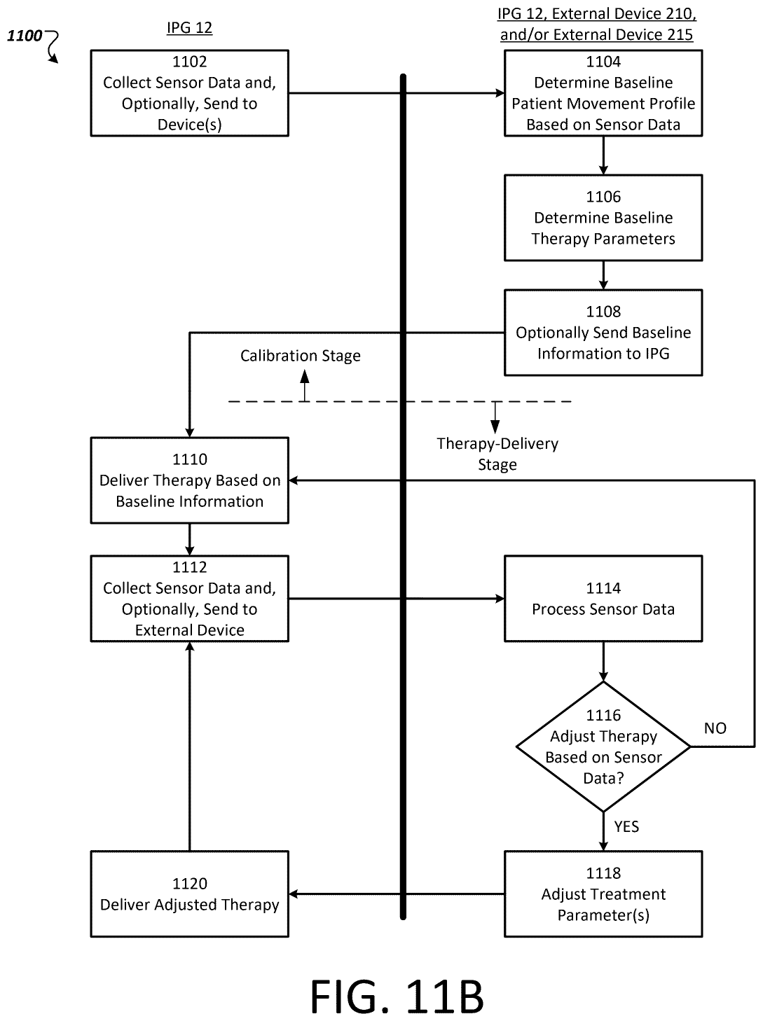

FIG. 11A illustrates a process 1000 of calibrating, delivering, and adjusting therapy to a patient according to an example. A lefthand side of the process 1000 may be executed by the IPG 12. A righthand side of the process 1000 may be executed by the external device 210. In some examples, the external device 210 may be a physician's computer. In some examples, one or more steps shown on the right-hand side of the process 1000 may be executed by the IPG 12 in lieu or in addition to the external device 210, as shown for example in FIG. 11B.

Implementation of the system 100 may occur over four stages. In an implantation stage, a patient is implanted with the implantable treatment system 10 via an implant procedure. In a recovery stage, the patient then heals and recovers for several weeks, such as 4-6 weeks. In a calibration stage, the patient returns to a physician's office to begin calibrating the system 10 once the patient has recovered appropriately. This calibration stage may occur over several weeks, such as one month. In a therapy-delivery stage, the system 10 begins delivering therapy. The therapy-delivery stage may last for an indefinite period of time, during which the patient may periodically return to the physician's office for regular check-ups. The physician may or may not adjust treatment parameters of the therapy during these check-ups.

The process 1000 is described as beginning at the calibration stage, where the system 10 has been implanted in the patient and the patient begins a calibration process. Acts 1002-1008 may comprise the calibration stage. Acts 1010-1020 may comprise the therapy-delivery stage.

At act 1002, the IPG 12 collects sensor data and sends the sensor data to the external device 210. Act 1002 may be executed over a calibration period of time to accumulate information indicative of a baseline state of the patient, that is, before therapy is applied. The calibration period of time may last several days or weeks (for example, one month) during which the IPG 12 is collecting sensor data. During the calibration period of time, the IPG 12 may not deliver any stimulation energy to the patient. Thus, the sensor data collected over the calibration period of time may be indicative of the patient's sleeping characteristics, such as arousals or position changes, in the absence of therapy delivery. In this manner, the IPG 12 and/or the external computing device 210 may identify sleeping characteristics, such as arousals or position changes, that are attributable to therapy delivery parameters.

Sensor data may include data sensed by the sensors 18, 19, and 20. The IPG 12 may use the sensor data to establish a baseline patient movement profile indicating a baseline amount of patient movement before therapy begins. For example, the sensor data may include data from the at least one accelerometer 36 indicating patient movement. The sensor data may also include breathing-rate information from the sensors 18, 19, and/or 20 (for example, including transthoracic-impedance sensors). The IPG 12 may send all or a portion of the collected sensor information to the external device 210 via a wireless-communication technology.

At act 1004, the external device 210 receives the collected sensor data and determines a baseline patient movement profile based on the collected sensor data.

The baseline patient movement profile may indicate baseline movement metrics for the patient in the absence of stimulation therapy and while sleeping where sleeping corresponds to a degree of arousal less than full arousal (for example, no arousal, mild arousal, and/or moderate arousal). For example, the baseline movement profile may indicate an average number of times that the patient rolls in a given sleeping time period, a frequency of rolls, an average cumulative number of times that a patient rolls in quick succession when the patient does roll, a magnitude of each roll (for example, expressed as a number of angular degrees rolled at once), an amount of time that the patient spends in each position, a range of pitch angles associated with the patient being asleep, a range of pitch angles associated with the patient being awake and sitting up, an average number of times that the patient wakes up during a sleeping time period (for example, between falling asleep for the night and waking up for the day), a number and degree of arousals, and/or other information related to a position and/or movement of the patient.

At act 1006, the external device 210 determines baseline treatment parameters for the patient. In some examples, the physician may provide the baseline treatment parameters. In other examples, the external device 210 may determine the baseline treatment parameters with or without input from the physician. For example, the external device 210 (and/or IPG 12) may determine the baseline treatment parameters based on the sensor data with or without input from the physician.

Baseline treatment parameters include baseline values for the treatment parameters that determine the electrical parameters of a stimulation waveform provided by the IPG 12. As discussed above, the IPG 12 may provide different stimulation energies to one or more nerves, such as a phrenic nerve, a hypoglossal nerve, an ansa cervicalis nerve, and so forth. Accordingly, while the following discussion of example treatment parameters may refer to a singular stimulation waveform or stimulation energy, it is to be appreciated that different treatment parameters may be established for different stimulation waveforms or stimulation energies. For example, a different minimum effective energy may be established for upper-airway nerve stimulation than for phrenic nerve stimulation.

Treatment parameters may include a minimum effective energy. As discussed above, the minimum effective energy may include a value of energy below which therapy is not effective. For example, in the context of providing phrenic nerve stimulation, the minimum effective energy may be an energy below which the diaphragm is not modulated. The physician may determine the minimum effective energy by observing a patient response to the IPG 12 providing a stimulation energy. For example, the physician may observe a response by the patient's diaphragm to the stimulation energy.

Similarly, in the context of providing upper-airway nerve stimulation, the minimum effective energy may be an energy below which airway patency is not improved. The physician may determine the minimum effective energy by observing (visually and/or via palpation) a patient response to the IPG 12 providing a stimulation energy. For example, the physician may observe a response by the patient's tongue and a degree of protrusion to the stimulation energy as a stimulation energy is provided to a hypoglossal nerve. When the hypoglossal nerve is stimulated the tongue lifts and protrudes. The degree of protrusion can be an indicator of minimum effectiveness of a stimulation energy. In an implementation, the physician may observe a physical response to the stimulation energy as a stimulation energy is provided to an ansa cervicalis nerve. The ansa cervicalis nerve stimulates strap muscles in the neck. The minimum effective energy may be the minimum energy that improves the patency of the patient's open or partially open airway. In various examples, the physician may determine a minimum effective energy in several sleeping positions, such as a right-side position, left-side position, prone position, and/or supine position.

Treatment parameters may also include minimum and maximum treatment energy values that together with the minimum effective energy define one or more titration ranges for the stimulation energy. The minimum and maximum treatment energy values may be greater than the minimum effective energy or the minimum treatment energy value may be equal to the minimum effective energy with the maximum treatment energy value greater than the minimum effective energy.

The external computing device 210 may program the IPG 12 with treatment parameters including the minimum and maximum treatment energy values. The external computing device 210 may receive physician input and base the treatment parameters on the physician input. In some examples, the external computing device 210 may be physically located in a physician's office and communicate with the IPG 12 during a visit by the patient to the physician's office. For example, communication may be achieved via electromagnetic telemetry. In some examples, the external computing device 210 may be physically located in a patient's home or other sleeping location. The IPG 12 and the external computing device 210 may include transceivers configured to communicate and exchange data via a Bluetooth® protocol. The physician or other care provider may provide input and/or receive data from the external computing device 210 via a remote communications link such as a telemedicine link based on an Internet Protocol (for example, a Wi-Fi protocol according to an IEEE 802.11 standard).

In an implementation, minimum and maximum treatment energy values may depend at least in part on patient feedback while the patient is awake. For example, the physician may adjust the treatment energy value over an energy range and observe a range of muscular responses (for example, response of the diaphragm and/or the patient's tongue). The physician and/or a sensor may observe and/or measure the strength and/or quality of muscle contractions based, for example, on the strength and/or quality of air flow and breathing of the patient. In an example, a sensor configured to measure air flow and breathing may be communicatively coupled to the external computing device 210 and/or the IPG 12. The patient may offer feedback as to how they feel in response to the stimulation energy being applied according to a degree of sensation (for example, a weak, medium, or strong sensation). The physician may correlate the muscular responses with the degree of sensation to determine minimum effective energy, minimum treatment energy, and/or maximum treatment energy based on an optimization of physical response with patient sensation. This optimization maximizes muscular response while minimizing patient sensation.

The physician may provide input to the external computing device 210 regarding this optimization such that the external computing device 210 can program the IPG 12 to deliver stimulation accordingly. Over time, these stimulation energy ranges may change. For example, as a patient may gradually adjust to the stimulation energy so that discomfort at a particular energy level may decrease over time enabling a higher stimulation energy that increases muscular response to provide more effective therapy. Additionally, as a patient adjusts to the provision of phrenic nerve stimulation and more readily entrains, the upper airway stimulation needs may change thus affecting the minimum and maximum treatment energies. As a further consideration, the minimum and maximum treatment energies may vary with changes in a patient's habitus, changes in cardiovascular condition, and/or changes in other medical conditions.

The external computing device 210 may program the IPG 12 to automatically increase the minimum or maximum treatment energies over time. For example, the external computing device 210 may program the stimulation energy parameter(s) such that the minimum or maximum treatment energies increase over the course of one night, over the course of a week, over the course of a month, and so forth. The external computing device 210 may also program the IPG 12 to decrease the minimum or maximum treatment energies in response to certain conditions. In one example, the external computing device 210 may program the IPG 12 to decrease the minimum and/or maximum treatment energies if, for example, the patient is entrained at or near the minimum stimulation energy, which may indicate that the stimulation energy is higher than necessary to achieve entrainment. In another example, the external computing device 210 may program the IPG 12 to decrease the minimum and/or maximum treatment energies if, for example, the patient is too aroused by the stimulation energy. For example, if the patient's movement deviates substantially from the baseline patient movement profile, the minimum and maximum treatment energies may be reduced. The external computing device 210 may set a different minimum and maximum treatment energy for different sleeping positions in some examples.

Treatment parameters may also include a magnitude, frequency, and/or duration of each energy step. As discussed above, the IPG 12 may ramp the stimulation energy up over a ramping-up period. The increase in energy over this ramping-up period may be referred to as a step. Increasing the energy over time may include ramping up the energy over several energy steps. A time between successive steps is the inverse of the step frequency. The external computing device 210 may select the number, frequency, and/or magnitude of energy steps for the IPG 12 to implement while titrating the therapy between a minimum and maximum value. Increasing the magnitude and/or frequency of the energy steps may increase the rate at which energy is ramped up, which may increase a chance of arousing the patient. The external computing device 210 and/or the IPG 12 may monitor patient response to the magnitude and/or frequency of the energy steps to determine a number, frequency, and/or magnitude over which to ramp up the stimulation energy that increases the stimulation energy in a sufficiently short amount of time without ramping the energy up so quickly that the patient is undesirably aroused. The external computing device 210 may program the IPG 12 to increase energy over energy steps with irregular parameters. For example, the physician may program the IPG 12 to step up energy with an incremental decrease in the magnitude of the steps as the stimulation energy reaches a maximum treatment energy value and/or an incremental increase in the quantity of steps as the stimulation energy approaches the maximum treatment energy.

Treatment parameters may also include a frequency at which a therapy-effectiveness metric is calculated, if at all. For example, in providing phrenic nerve stimulation, the IPG 12 and/or the external computing device 210 may calculate an entrainment index to compare against at least one entrainment-index threshold. The external computing device 210 may select a frequency for the entrainment index calculation. In providing upper-airway nerve stimulation, the IPG 12 may calculate an airway-patency metric to compare against at least one airway-patency-metric threshold.

Treatment parameters may also include one or more therapy-effectiveness thresholds or ranges. Therapy-effectiveness thresholds or ranges may include thresholds or ranges above, below, or within which some action is taken. For example, the IPG 12 may implement multiple entrainment-index thresholds. If the entrainment index is below a first threshold, the patient may be considered not entrained. If the entrainment index is above the first threshold, the patient may be considered entrained. However, even if the entrainment index is above the first threshold, the IPG 12 may take different actions depending on a value of the entrainment index. The external computing device 210 may establish various entrainment-index ranges and associated actions to take in response to the entrainment index falling within the entrainment-index ranges. For example, suppose that a patient is considered entrained with an entrainment index above 70%. The external computing device 210 may specify that if the entrainment index falls between 70% and 80%, the stimulation energy will be maintained at its present value. The external computing device 210 may further specify that if the entrainment index falls between 80% and 90%, the stimulation energy will be stepped down if the entrainment index remains between 80% and 90% for at least a threshold period of time, such as five minutes. The external computing device 210 may further specify that if the entrainment index is between 90% and 100%, the stimulation energy will be stepped down immediately and the minimum and maximum treatment energy values will be decreased. In other examples, other therapy-effectiveness metrics, numerical ranges, and/or responses to the therapy-effectiveness metric falling within a given range are within the scope of the disclosure.

Treatment parameters may also include responses to patient arousal. Patient arousal may be quantified by a number of angular degrees rolled and/or movement between positional quadrants. The IPG 12 may respond to patient arousal if, for example, the patient movement deviates from the baseline patient movement profile. The external computing device 210 may specify the IPG's 12 response to the patient movement deviating from the baseline patient movement profile. For example, the external computing device 210 may program the IPG 12 to reduce the stimulation energy and/or the minimum and maximum treatment energy values in response to patient arousal. The external computing device 210 may specify several tiered responses that the IPG 12 may implement in response to the patient movement deviating from the baseline patient movement profile. For example, the external computing device 210 may program the IPG 12 to reduce the stimulation energy if the patient is mildly aroused (such as by deviating slightly from a baseline patient movement profile), but may program the IPG 12 to reduce the stimulation energy and the minimum and maximum treatment energy values if the patient is moderately aroused (such as by deviating more significantly from the baseline patient movement profile). Deviating slightly from the baseline patient movement profile may include, for example, rolling one or two more times than normal in a given period of time (for example, five minutes). Deviating more significantly from the baseline patient movement profile may include, for example, rolling three or more times than normal in a given period of time. These are non-limiting examples; in other examples, the external computing device 210 may program the IPG 12 to take any desired response to patient arousal, and may or may not program the IPG 12 to scale up the IPG's 12 response with the amount of patient arousal.

At act 1008, the external computing device 210 sends the baseline information to the IPG 12. The baseline information includes the baseline patient movement profile and the baseline treatment parameters.

Acts 1002-1008 may be executed during a calibration phase. In some examples, the IPG 12 may not provide any electrical stimulation during the calibration phase. In other examples, the IPG 12 may provide electrical stimulation during the calibration phase, but only so that the IPG 12 and/or the external computing device 210 can collect data to monitor the patient's response to the electrical stimulation and calibrate treatment parameters accordingly. After act 1008, a therapy-delivery stage begins.

At act 1010, the IPG 12 begins to deliver therapy during regular sleep cycles of the patient based on the baseline information programmed as described above. For example, the processor 30 may cause the stimulation circuitry 24 to deliver and titrate stimulation energy generated by the power source 27. As discussed above, the baseline information may include baseline treatment parameters including minimum effective energies, minimum treatment energies, and maximum treatment energies for the patient either in any sleeping position or in multiple sleeping positions. The baseline treatment parameters may dictate how the IPG 12 titrates the stimulation energy within the minimum and maximum treatment energies. The baseline patient movement profile may indicate a baseline amount of patient movement such that movement in excess of the baseline amount of patient movement may be attributed to the stimulation energy and the patient may be considered aroused. The baseline treatment parameters may dictate how the IPG 12 responds to different degrees of arousal in titrating the stimulation energy.

At act 1012, the IPG 12 collects sensor data. The IPG 12 may collect sensor data as the IPG 12 provides therapy. The sensor data may include sensor data received from the sensors 18, 19, and/or 20. Sensor data may include any information relevant to the therapy, including patient-movement information, patient-position information, and/or information indicative of at least one therapy-effectiveness metric (such as breathing-rate information indicative of an entrainment index). The sensor data may be received from sensors such as motion sensors (for example, accelerometers), pressure sensors, acoustic sensors, transthoracic-impedance sensors, light sensors, airflow sensors, microphones, ultrasonic transducers, heart rate sensors, blood oxygenation sensors (for example, pulse oximeters), muscular electrical activity sensors (for example, electromyography sensors), peripheral arterial tone sensors, and/or other types of sensors.

The IPG 12 sends the sensor data to the external device 210 at act 1012. The external device 210 may collect the sensor data, such as via a wireless communication connection between the IPG 12 and the external device 210. Accordingly, the IPG 12 may execute acts 1010 and 1012 over the course of several days or weeks after the calibration stage.

At act 1014, the computing device 210 processes the sensor data. The computing device 210 may process the sensor data to determine information which may be used to evaluate and/or adjust the treatment parameters. For example, the sensor data may include patient-movement information and/or patient-position information which the computing device 210 may process to determine information such as an amount of time spent in each sleeping position, an amount of arousal compared to the baseline arousal, a frequency at which the patient is aroused by energies above a certain threshold, or any other information which may be determined based on the sensor data.

At act 1016, a determination is made as to whether to adjust the treatment parameters based on the processed sensor data. In some examples, the external computing device 210 and/or the IPG 12 may evaluate adjustments of the treatment parameters and re-program or re-configure the IPG 12 accordingly. In various examples, the computing device 210 automatically determines whether to adjust the treatment parameters with full, partial, or without input from the physician.

The external computing device 210 may adjust the treatment parameters if, for example, the patient is routinely being aroused by the stimulation energy. For example, if the patient is waking up several more times per night than during the baseline period, then the external computing device 210 may decrease the minimum and/or maximum treatment energy values. The external computing device 210 may also adjust the treatment parameters if, for example, the patient is still experiencing substantial symptoms of one or more breathing disorders. For example, if the patient is regularly experiencing CSA and/or OSA events that disturb the patient's sleep, then the external computing device 210 may increase the minimum and/or maximum treatment energy values.

In some examples, the external computing device 210 may analyze a number of rolls within various energy bins, or ranges. Bins with particularly high numbers of rolls may be causing a patient too much arousal. The external computing device 210 may adjust the treatment parameters of the IPG 12 to avoid these energy ranges, or to ramp up more slowly to these energy ranges. In other examples, the external computing device 210 may adjust treatment parameters for other reasons.

An external computing device 210 may also evaluate a likelihood that the stimulation is causing the patient to be aroused in some examples. For example, the IPG 12 may monitor an amount of time between adjusting (for example, increasing) a stimulation energy and when the patient next rolls. Longer times between the energy adjustment and the patient rolling may correlate with a lower likelihood that the patient rolling was caused by the energy adjustment. Shorter times between the energy adjustment and the patient rolling may correlate with a higher likelihood that the patient rolling was caused by the energy adjustment. For example, if the patient rolls immediately after the stimulation energy is increased, it may be more likely that the patient arousal was caused by the energy increase.

If a determination is made that the treatment parameters should not be adjusted (1016 NO), then the process 1000 returns to act 1010. The IPG 12 may continue delivering therapy based on the unmodified treatment parameters at act 1010.

Otherwise, if a determination is made that the treatment parameters should be adjusted (1016 YES), then the process continues at act 1018.

At act 1018, one or more treatment parameters are adjusted. As noted above, treatment parameters may be adjusted for various reasons. In some examples, a physician may utilize the computing device 210 to adjust one or more treatment parameters, and the computing device 210 may reprogram or reconfigure the IPG 12 to adjust the one or more treatment parameters.

At act 1020, the IPG 12 delivers adjusted therapy with the adjusted treatment parameters. For example, the IPG 12 may continue to deliver therapy when the patient is asleep, but pursuant to different treatment parameters, such as different minimum and/or maximum treatment energy values. Act 1012 may then be repeated, with the IPG 12 collecting sensor data indicative of the adjusted therapy. The IPG 12 may collect sensor data over the course of several days. Subsequently, the computing device 210 may again process the sensor data (act 1014) and the external computing device 210 may determine whether to re-adjust the treatment parameters (act 1016). The process 1000 may therefore be repeatedly executed as the patient receives therapy for some period of time.

In other examples, aspects of the process 1000 may be executed by other devices. In one example, all aspects of the process 1000 may be executed by the IPG 12. For example, the IPG 12 may determine baseline information, deliver therapy, and routinely re-evaluate therapy and potentially adjust the treatment parameters, all without any input from the external device 210. In another example, some aspects (including all aspects or fewer than all aspects) of the process 1000 described as being performed by the external device 210 may be performed by the IPG 12 and/or the patient-specific external device 215 in addition to, or in lieu of, the external device 210.

For example, the patient-specific external device 215 may include a patient's smartphone, laptop, tablet, or other device, which the patient may use to perform one or more aspects of the process 1000 described as being performed by the external device 210. For example, the patient may be able to use their smartphone to adjust certain treatment parameters, such as by decreasing a maximum treatment energy provided by the IPG 12. In this example, the patient may provide input to the patient-specific external device 215 to send wireless signals to the IPG 12 to adjust one or more treatment parameters.

In an example in which the patient-specific external device 215 is a patient's smartphone, it may also be advantageous for the patient's smartphone to execute some aspects of the process 1000 to leverage the processing power of the patient's smartphone. For example, because the IPG 12 may have limited processing power, the patient may use their smartphone to perform certain calculations or other processes in lieu of, or in addition to, the IPG 12. The IPG 12 may communicate with the patient's smartphone via a wireless connection to exchange any relevant information, and the patient's smartphone may perform applicable calculations.

FIG. 11B illustrates a process 1100 of calibrating, delivering, and adjusting therapy to a patient according to an example. The process 1100 is substantially similar to the process 1000, except that certain aspects of the process 1000 which are executed by the external device 210 are instead executed by one or more of the IPG 12, the external device 210, or the patient-specific external device 215 in the process 1100. A lefthand side of the process 1000 may still be executed by the IPG 12. A righthand side of the process 1000 may be executed by the IPG 12, the external device 210, the patient-specific external device 215, or combinations thereof.

Act 1102 may be similar to act 1002 and includes the IPG 12 collecting sensor data during a calibration stage. The calibration period of time may last several days or weeks (for example, one month) during which the IPG 12 is collecting sensor data. During the calibration period of time, the IPG 12 may not deliver any stimulation energy to the patient. Thus, the sensor data collected over the calibration period of time may be indicative of the patient's condition without any therapy. In some examples, the IPG 12 may deliver stimulation energy to the patient during the calibration period for the purpose of evaluating a baseline condition of the patient.

Act 1102 may differ from act 1002 at least in that act 1102 may or may not include sending the sensor data to another device. In some examples of act 1102, the IPG 12 sends the sensor data to the external device 210 and/or the patient-specific external device 215. In other examples of act 1102, the IPG 12 does not send the sensor data to any other devices. For example, if the IPG 12 executes acts 1104, 1106, and 1108 (or if 1108 is not executed, as discussed below), it may not be necessary or desirable for the IPG 12 to send the sensor data to any other device. In some examples, the IPG 12 may execute all aspects of the righthand side of the process 1100, in which case the IPG 12 may or may not send information to other devices.

Act 1104 may be similar to act 1004 and includes one or more of the IPG 12, the external device 210, or the patient-specific external device 215 determining a baseline patient movement profile based on the sensor data. If act 1104 is executed by the IPG 12, then the IPG 12 may not send the sensor data collected at act 1102 to any other device, and may use the sensor data to determine the baseline patient movement profile. If act 1104 is executed by the external device 210 and/or the patient-specific external device 215, act 1102 may include the IPG 12 sending the sensor data to the external device 210 and/or the patient-specific external device 215 such that the external device 210 and/or the patient-specific external device 215 may determine a baseline patient movement profile based on the sensor data.

Act 1106 may be similar to act 1006 and includes one or more of the IPG 12, the external device 210, or the patient-specific external device 215 determining baseline treatment parameters. In some examples, one or more of the IPG 12, the external device 210, or the patient-specific external device 215 may determine the baseline treatment parameters based on pre-determined parameter values. In various examples, one or more of the IPG 12, the external device 210, or the patient-specific external device 215 may determine the baseline treatment parameters based on the sensor data, which may be provided by the IPG 12 at act 1102. If the external device 210 and/or the patient-specific external device 215 determines the baseline treatment parameters at act 1106 based on sensor data, act 1102 may include the IPG 12 sending the sensor data to the devices 210, 215.

At optional act 1108, the external device 210 and/or the patient-specific external device 215 may send baseline information including one or more of the baseline patient movement profile and the baseline treatment parameters to the IPG 12. Act 1108 may be executed if either of the devices 210, 215 determines the baseline treatment parameters at act 1106. Otherwise, if only the IPG 12 executes both acts 1104 and 1106, then optional act 1108 may not be executed because the IPG 12 may already have all baseline information.

Act 1110 may be similar to act 1010, and includes the IPG 12 delivering therapy based on baseline treatment parameters. Act 1110 may be substantially identical to act 1010, although the baseline treatment parameters may have been generated by either the IPG 12 or the patient-specific external device 215.

Act 1112 may be similar to act 1012, and includes the IPG 12 collecting sensor data as the IPG 12 delivers therapy. Act 1112 may optionally include the IPG 12 sending the sensor data to the external device 210 and/or the patient-specific external device 215. For example, act 1112 may include the patient going to a physician's office for a check-up during which the IPG 12 may provide the sensor data to the external computing device 210. In another example, act 1112 may include the patient using the patient-specific external device 215 (for example, the patient's smartphone) to receive the collected sensor data from the IPG 12. In still other examples, act 1112 may only include the IPG 12 collecting the sensor data and may not include the IPG 12 sending the sensor data to any other device.

Act 1114 may be similar to act 1014, and includes a device processing the sensor data. Whereas act 1014 may be executed by the external device 210, act 1114 may include one or more of the IPG 12, the external device 210, or the patient-specific external device 215 processing the sensor data. If the external device 210 or the patient-specific external device 215 executes act 1114, act 1112 may include the IPG 12 sending the sensor data to the external device 210 and/or the patient-specific external device 215.

Act 1116 may be similar to act 1016, and includes determining whether to adjust treatment parameters. In some examples, one or more of the IPG 12, the external device 210, and/or the patient-specific external device 215 may automatically determine whether to adjust the treatment parameters. In various examples, a physician may provide inputs to the external device 210 indicating whether and in what way to change the treatment parameters. In at least one example, a patient may provide inputs to the patient-specific external device 215 indicating patient feedback which may be used to determine whether and in what way to change the treatment parameters. If the treatment parameters are not adjusted (1116 NO), then the process 1100 returns to act 1110. If the treatment parameters are adjusted (1116 YES), the process 1100 continues to act 1118.

Act 1118 may be similar to act 1018, and one or more of the IPG 12, the external device 210, and/or the patient-specific external device 215 may adjust one or more treatment parameters as decided at act 1116. In some examples, the same device determines that the treatment parameters should be adjusted (act 1116 YES) and adjusts the treatment parameters (act 1118). In other examples, one device determines that the treatment parameters should be adjusted and another device adjusts the treatment parameters.

Act 1120 may be substantially similar to act 1020, and includes the IPG 12 delivering adjusted therapy to a patient. The process 1100 then returns to act 1112.

Accordingly, the IPG 12 may execute certain acts of the process 1100, such as collecting data from the sensors 18, 19, and/or 20 during calibration (act 1102), delivering therapy (act 1110) and adjusted therapy (act 1120), and collecting data from the sensors 18, 19, and/or 20 during therapy delivery (act 1112). Other acts of the process 1100, which may be more primarily concerned with processing data, may be executed by any of the IPG 12, the external device 210, and/or the patient-specific external device 215.

FIG. 12 illustrates a process 1200 of providing sleep disordered breathing treatment for a patient according to an example. The process 1200 may be executed at least in part by the IPG 12. Accordingly, examples may be provided according to an implementation in which the IPG 12 executes aspects of the process 1200.

At act 1202, at least one of the leads 13 receives an electrical stimulation energy. In some examples, the phrenic nerve stimulation lead 14 receives a stimulation energy to deliver to a phrenic nerve, and/or the airway nerve stimulation lead 16 receives another stimulation energy to deliver to an airway nerve, such as a hypoglossal nerve or ansa cervicalis nerve. The leads 13 may receive stimulation energy from the stimulation circuitry 24. For example, the processor 30 may cause the stimulation circuitry 24 to provide a stimulation energy generated using power derived from the power source 27. The processor 30 may cause the stimulation circuitry 24 to provide the stimulation energy having parameters indicated by treatment parameters stored in a location accessible to the processor 30, such as the memory 28. For example, the memory 28 may store a patient profile accessible to the processor 30.

At act 1204, at least one of the leads 13 delivers the stimulation energy received at act 1202 to at least one target nerve. For example, the phrenic nerve stimulation lead 14 may deliver a first stimulation energy to a phrenic nerve (that is, the phrenic nerve may be a target nerve) and/or the airway nerve stimulation lead 16 may deliver a second stimulation energy to an airway nerve, such as a hypoglossal nerve (that is, the hypoglossal nerve may be a target nerve) and/or an ansa cervicalis nerve (that is, the ansa cervicalis nerve may be a target nerve). The leads 13 may include or be coupled to one or more nerve cuffs and/or transvenous leads to deliver the electrical stimulation energy to at least one target nerve.

At act 1206, the IPG 12 receives at least one signal indicative of a change in at least one sleep parameter from at least one of the sensors 18, 19, and/or 20. For example, the processor 30 may receive at least one signal. A sleep parameter may include patient-movement, patient-position, and/or degree of arousal information. The at least one accelerometer 36 may provide at least one signal with accelerometer data which is indicative of a change in the patient's position, movement, and/or degree of arousal. At least one signal may therefore include an acceleration signal from the at least one accelerometer 36. In other examples, at least one sleep parameter may include additional or different information in addition to, or in lieu of, the patient-movement and/or patient-position information. For example, at least one sleep parameter may include transthoracic-impedance information received from the external sensors 18 (for example, from a transthoracic-impedance sensor) which the processor 30 may analyze to determine a change in at least one sleep parameter.

In examples in which at least one signal includes at least one acceleration signal, at least one signal may be indicative of patient-movement, patient-position, and/or degree of arousal information, which may include patient rolling information. Patient rolling information may include a number of rolls, a frequency of rolls (for example, a number of rolls within a certain period of time), a magnitude of rolls (for example, movement of the patient relative to positional quadrants, such as from the prone position to a side position or from the prone position to a supine position), a cumulative number of rolls in a respective time period, or combinations thereof.

At act 1208, the IPG 12 determines the change in at least one sleep parameter based on at least one signal. For example, the processor 30 may process at least one signal to determine the change in at least one sleep parameter. For example, determining the change in at least one of patient position or patient movement may include determining a degree of arousal or a change of degree of arousal of the patient based on the change in at least one of patient movement or position.

Determining the degree of arousal may include comparing the change in at least one sleep parameter, or information derived therefrom, to one or more respective thresholds. For example, if the change in at least one sleep parameter is indicative of patient-movement information, act 1208 may include comparing a number of rolls, a frequency of rolls, a magnitude of rolls, a cumulative number of rolls in a respective time period, or combinations thereof, to one or more respective thresholds (for example, a threshold number of rolls, a threshold frequency of rolls, and so forth). Thus, the IPG 12 may determine that the patient is exhibiting a first degree of arousal if one or more of a number of rolls, a frequency of rolls, a magnitude of rolls, a cumulative number of rolls in a respective time period, or combinations thereof subceed (that is, are below) respective thresholds, and may determine that the patient is exhibiting a second degree of arousal if one or more of a number of rolls, a frequency of rolls, a magnitude of rolls, a cumulative number of rolls in a respective time period (which may differ from the respective time period during which the cumulative number of rolls are monitored for the first degree of arousal), or combinations thereof exceed respective thresholds.

As an example, the IPG 12 may compare a magnitude of the patient's rolls relative to the positional quadrants 402-408. A first degree of arousal may correspond to patient motion within a single one of the quadrants 402-408. For example, if the patient rolls within the supine quadrant 402 but does not roll out of the supine quadrant 402, the IPG 12 may determine that the patient is exhibiting a first degree of arousal. The first degree of arousal may be relatively weak, because the patient is not rolling a substantial amount.

A second degree of arousal may correspond to patient motion between the positional quadrants 402-408. For example, if the patient rolls from the supine quadrant 402 to the left-side quadrant 408, the IPG 12 may determine that the patient is exhibiting a second degree of arousal. The second degree of arousal may be relatively stronger, because the patient is rolling a more substantial amount than in the first degree of arousal. In some examples, the IPG 12 may determine that the patient is experiencing more arousal if the patient moves between more than two positional quadrants. For example, if the patient rolls approximately 180° from the left-side quadrant 408 to the supine quadrant 402 and finally to the right-side quadrant 406, the IPG 12 may determine that the patient is experiencing a higher degree of arousal than if the patient simply rolled approximately 90° from the left-side quadrant 408 to the supine quadrant 402. In other examples, the IPG 12 may determine that the patient is equally aroused if the patient rolls between two positional quadrants or three or more positional quadrants.

In other examples, the IPG 12 may determine a degree of arousal without consideration of movement between or within positional quadrants. For example, the IPG 12 may evaluate a number of angular degrees rolled in a given period of time regardless of whether or not the patient moves between positional quadrants. A third degree of arousal may correspond to a number of angular degrees rolled in a given period of time (for example, beginning when the patient begins rolling and ending when the patient stops rolling) irrespective of any positional quadrants. In some examples, the IPG 12 may determine the third degree of arousal by comparing the patient motion (for example, a number of angular degrees rolled) to a threshold (for example, a threshold number of angular degrees) and determining that the patient is exhibiting a third degree of arousal if the patient motion exceeds the threshold amount.

In some examples, the IPG 12 may determine a degree of arousal by determining a deviation from a baseline patient movement profile. As discussed above, the baseline patient movement profile may be determined during a calibration phase (for example, at acts 1004 and/or 1104) before therapy is applied. Once therapy is applied, deviations between the patient's present state of arousal (that is, arousal while therapy is applied) and the patient's baseline state of arousal (that is, arousal observed during the calibration phase before therapy is applied) may be attributed to the therapy.

In one example, the deviation from the baseline patient movement profile may include a present number of rolls over a present rolling window of time indicated by at least one sleep parameter exceeding or subceeding a baseline number of rolls over a baseline rolling window of time by at least a threshold number of rolls. Rolls may be defined as movement between the quadrants 402-408. The baseline rolling window of time may be equal to the present rolling window of time. For example, suppose that the rolling windows of time are equal to one hour. The baseline number of rolls may include an average or maximum number of rolls over a one-hour rolling window of time observed during the calibration stage. Act 1208 may therefore include the IPG 12 repeatedly comparing a presently observed number of rolls over the last hour to the average or maximum number of rolls observed over a one-hour period during calibration. For example, if the maximum number of rolls observed in a one-hour time window during calibration is six rolls, but the IPG 12 determines during a present time that the patient has rolled seven or more times in the past hour, then the IPG 12 may determine that the patient is deviating from the baseline patient movement profile.

In another example, the deviation from the baseline patient movement profile may include a present frequency of rolls indicated by at least one sleep parameter exceeding or subceeding a baseline frequency of rolls by at least a threshold frequency of rolls. Each frequency may be evaluated over the same period of time, such as since the patient fell asleep. The baseline frequency of rolls may include an average or maximum frequency of rolls observed during the calibration stage. Act 1208 may therefore include the IPG 12 repeatedly comparing a presently observed frequency of rolls to the average or maximum frequency of rolls observed during calibration. For example, if the maximum frequency of rolls observed during calibration is four rolls per hour, but the IPG 12 determines during a present time that the patient is rolling at a frequency of five or more rolls per hour, then the IPG 12 may determine that the patient is deviating from the baseline patient movement profile.

In another example, the deviation from the baseline patient movement profile may include a present magnitude of rolls indicated by at least one sleep parameter exceeding or subceeding a baseline magnitude of rolls by at least a threshold magnitude. The baseline magnitude of rolls may include an average or maximum magnitude of rolls (for example, expressed as a number of angular degrees rolled between when the patient first starts rolling and finally stops rolling) observed during the calibration stage. Act 1208 may therefore include the IPG 12 repeatedly comparing a presently observed magnitude of rolls to the average or maximum magnitude of rolls observed during calibration. For example, if the maximum magnitude of rolls observed during calibration is 200°, but the IPG 12 determines during a present time that the patient rolls 201° or more, then the IPG 12 may determine that the patient is deviating from the baseline patient movement profile.

In one example, the deviation from the baseline patient movement profile may include a present number of rolls (and/or a present cumulative number of rolls) over a present reference window of time (also referred to as a present reference period of time) indicated by at least one sleep parameter exceeding or subceeding a baseline number of rolls (and/or a baseline cumulative number of rolls) over a baseline reference window of time (also referred to as a baseline reference period of time) by at least a threshold number of rolls (and/or a threshold cumulative number of rolls). Rolls may be defined as movement between the quadrants 402-408. The baseline reference window of time may be equal to the present reference window of time. Reference windows of time may include specific times of day (for example 12 AM to 2 AM), specific days (for example, Monday through Sunday), or relative windows of time, such as an amount of time since the patient first fell asleep for a sleeping time period (for example, when the patient goes to bed for a full night's sleep). For example, suppose that the reference windows of time span from the patient falling asleep for the night and waking up for the day. The baseline number of rolls may include an average or maximum number of rolls observed during a single night of sleep. Act 1208 may therefore include the IPG 12 repeatedly comparing a presently observed number of rolls since the patient fell asleep to the average or maximum number of rolls observed while the patient was asleep during calibration. For example, if the maximum number of rolls observed in a single night during calibration is 30 rolls, but the IPG 12 determines during a present time that the patient has rolled 31 or more times since falling asleep, then the IPG 12 may determine that the patient is deviating from the baseline patient movement profile.

In some examples, the IPG 12 may adjust the thresholds over time or may apply different thresholds over time. For example, thresholds may start as the minimum, average, maximum, or some other quality of what is observed during calibration, but may be modified over time to a different value. In some examples, the thresholds may be reduced. In other examples, the thresholds may be increased.

In various examples, a degree of arousal may indicate that the patient is awake. For example, if the IPG 12 determines, based on information from the at least one accelerometer 36, that the patient is pitching forward (for example, sitting up), the IPG 12 may determine that the patient is awake. The pitch angle may be determined relative to when the patient is lying down and sleeping. Determining that the patient is awake may be a maximum degree of arousal.

At act 1210, the IPG 12 causes the stimulation circuitry 24 to titrate the stimulation energy based on the change in at least one sleep parameter and/or based on the target nerve. Titrating the stimulation energy may include one or more adjustments of the electrical stimulation energy between a minimum effective energy and a target maximum treatment energy. The value of the minimum effective energy may depend at least in part on which target nerve is being targeted with the stimulation energy. As discussed above, a different minimum effective energy may be associated with each nerve, and in some examples a different minimum effective energy may be associated with each sleeping position. The IPG 12 may titrate the stimulation energy based on the presence or absence of patient arousal. As discussed above, determining at act 1208 that the patient is aroused may include the IPG 12 detecting a deviation from the baseline patient movement profile. Detection of a deviation of the rolling information from the baseline patient movement profile may therefore trigger the titration of the stimulation energy.

As discussed above with respect to FIGS. 6-9, in some examples, the IPG 12 may decrease the stimulation energy to a value equal to or greater than a minimum effective energy. For example, if the IPG 12 determines at act 1208 that the patient is aroused, the IPG 12 may cause the stimulation circuitry to decrease the stimulation energy to a value equal to or greater than the minimum effective energy. The one or more adjustments to the stimulation energy may therefore include a decrease from a first stimulation energy to a second stimulation energy in response to an increase in the degree of arousal, where the second energy is non-zero and greater than or equal to the minimum effective energy.

In various examples, the IPG 12 may increase the stimulation energy. For example, if the IPG 12 determines at act 1208 that the patient is not aroused, the IPG 12 may cause the stimulation circuitry to increase the stimulation energy to a value equal to or less than a maximum target energy. Determining that the patient is not aroused may include detecting an absence of a change in the degree of arousal; that is, determining that the patient is not aroused includes not determining that the patient is aroused. The one or more adjustments to the stimulation energy may therefore include an increase from a first stimulation energy to a second stimulation energy in response to a lack of increase in the degree of arousal.

As discussed above, energy may be increased (and, in some examples, decreased) over a plurality of steps. Each step comprises a change in energy. The one or more adjustments to the stimulation energy may include adjusting a quantity and/or magnitude of each step of the plurality of steps. The quantity and/or magnitude of the steps may vary as the energy is titrated. The one or more adjustments to the stimulation energy may also include adjusting a hold time between steps, that is, a time between steps during which energy is maintained at a given value.

In some examples, energy steps may be equal and/or constant. For example, suppose that the IPG 12 titrates the stimulation current to increase from 3 mA to 4 mA. In one example, the IPG 12 may increase the stimulation current in a single 1 mA step from 3 mA to 4 mA. In another example, the IPG 12 may increase the stimulation current in two 0.5 mA steps from 3 mA to 3.5 mA, and from 3.5 mA to 4 mA.

In another example, the energy steps may be irregular. In one example, the one or more adjustments to the stimulation energy may include an incremental decrease in the magnitude of the energy steps as the stimulation energy approaches a target maximum treatment energy. For example, suppose that the IPG 12 titrates the stimulation current to increase from 3 mA to a target maximum treatment current of 4 mA. In one example, the IPG 12 may increase the stimulation current in three irregular and incrementally decreasing steps, from 3 mA to 3.6 mA, and from 3.6 mA to 3.9 mA, and from 3.9 mA to 4 mA. In another example, the one or more adjustments to the stimulation current may include an incremental increase in the magnitude of the current steps as the stimulation current approaches a target maximum treatment current. For example, suppose that the IPG 12 titrates the stimulation current to increase from 3 mA to a target maximum treatment current of 4 mA. In one example, the IPG 12 may increase the stimulation current in three irregular and incrementally increasing steps, from 3 mA to 3.1 mA, and from 3.1 mA to 3.7 mA, and from 3.7 mA to 4 mA.

The IPG 12 may titrate the stimulation energy according to various orders of magnitude. In some examples, the IPG 12 may increase and/or decrease the stimulation current by tenth(s) of milliamps steps (that is, 0.1 mA step, 0.2 mA step, and so forth up to 0.9 mA step). In other examples, the IPG 12 may increase and/or decrease the stimulation current by hundredth(s) of milliamps steps (that is, 0.01 mA step, 0.02 mA step, and so forth up to 0.09 mA step). The order of magnitude of the titration steps may depend on a target nerve and/or a type of stimulation electrode (for example, transvenous lead electrode or nerve cuff electrode).

Accordingly, act 1110 includes titrating the stimulation energy based at least on at least one sleep parameter, which may be indicative of patient arousal (or an absence of patient arousal). In some examples, the IPG 12 may receive at least one feedback signal indicative of respiration of the patient and titrate the therapy based on at least one feedback signal. For example, the IPG 12 may receive at least one feedback signal from the external sensor 18, which may include a transthoracic-impedance sensor to measure breathing-rate information. In other example, the IPG 12 may receive breathing information from one or more other sensors of the sensors 18, 19, and/or 20.

In some examples, the IPG 12 may use the respiration information to determine airway patency of the patient. Determining the airway patency of the patient may enable the IPG 12 to determine whether and to what degree OSA or combination apnea is being treated in the patient. In other examples, the IPG 12 may use the respiration information to determine respiratory synchronization of the patient. Determining the respiratory synchronization of the patient may include the IPG 12 determining an entrainment index, which may enable the IPG 12 to determine whether and to what degree cSA or combination apnea is being treated in the patient.

As discussed above, the IPG 12 may use the entrainment index by comparing the entrainment index to at least one threshold value or range. In some examples, multiple entrainment-index thresholds may be implemented. For example, a first range may correspond to the patient being un-entrained, a second range may correspond to the patient being entrained to a desired level, and at least one third range may correspond to the patient being more entrained than necessary. The first and second range may be separated by a first threshold, and the second and at least one third range may be separated by a second threshold.

If the entrainment index falls within the first range (that is, the entrainment index is less than the first threshold), then the IPG 12 may increase the stimulation energy to increase entrainment. If the entrainment index falls within the second range (that is, the entrainment index is greater than the first threshold but less than the second threshold), then the IPG 12 may maintain the stimulation energy to continue producing entrainment at a desired level. If the entrainment index falls within at least one third range (that is, the entrainment index is greater than the second threshold), then the IPG 12 may decrease the stimulation energy to decrease patient arousal without causing the patient to become un-entrained.

Accordingly, the one or more adjustments to the stimulation energy may include decreasing the stimulation energy responsive to the entrainment index being greater than the second threshold, and may include increasing the stimulation energy responsive to the entrainment index being less than the first threshold, and may include maintaining the stimulation energy if the entrainment index is within the second range. Thus, in titrating the stimulation energy to a target maximum treatment energy, the IPG 12 may increase and/or decrease the stimulation energy to a value at which the entrainment index is within the second range. The target maximum treatment energy may change over time as the entrainment index changes.

As discussed above, including with respect to act 1210, the IPG 12 may, in response to detecting a change in at least one sleep parameter, cause the stimulation circuitry 24 to titrate a stimulation energy. However, in other examples, the IPG 12 may cause the stimulation circuitry 24 to adjust other electrical parameters of a stimulation waveform provided to the leads 13. The stimulation waveform may be characterized by a stimulation voltage signal (and a stimulation energy). The voltage signal may include a pulse train including a plurality of pulses. Adjusting the electrical parameters may include adjusting one or more of a stimulation voltage, a stimulation pulse duration (that is, a pulse duration of each pulse of a stimulation pulse train), an electrical stimulation energy duty cycle (that is, a duty cycle of each pulse of the stimulation pulse train), an electrical stimulation energy frequency content, a stimulation pulse leading ramp slope (for example, if the pulses of the pulse train increase or decrease in a sloping ramp), a pulse amplitude, a number of pulses in a pulse train, a time between pulse trains, and/or a waveform of each pulse. In various examples, adjusting the electrical parameters other than stimulation energy may require or lead to adjusting parameters of the stimulation energy.

Various controllers and/or processors, such as the processor 30, may execute various operations discussed above. The processor 30 may also execute one or more instructions stored on one or more non-transitory computer-readable media, which the processor 30 may include and/or be coupled to, which may result in manipulated data. The non-transitory computer-readable media may include memory and/or storage. In some examples, the processor 30 may include one or more processors or other types of controllers. In one example, the processor 30 is or includes at least one processor. In another example, the processor 30 performs at least a portion of the operations discussed above using an application-specific integrated circuit tailored to perform particular operations in addition to, or in lieu of, a processor. As illustrated by these examples, examples in accordance with the present disclosure may perform the operations described herein using many specific combinations of hardware and software and the disclosure is not limited to any particular combination of hardware and software components. Examples of the disclosure may include a computer-program product configured to execute methods, processes, and/or operations discussed above. The computer-program product may be, or include, one or more controllers and/or processors configured to execute instructions to perform methods, processes, and/or operations discussed above.

Returning to FIG. 1, each of the leads 13 may include one or more transvenous leads and/or nerve cuffs having one or more electrodes in order to deliver electrical stimulation to a target nerve. For example, the one or more nerve stimulation leads 13 may include one or more phrenic nerve stimulation leads 14 and one or more airway nerve stimulation leads 16. The phrenic nerve stimulation leads 14 may deliver electrical stimulation to a phrenic nerve, the airway nerve stimulation leads 16 may deliver electrical stimulation to a hypoglossal nerve, and/or the airway nerve stimulation leads 16 may deliver electrical stimulation to an ansa cervicalis nerve In some examples, the implantable treatment system 10 may include one or more distributed sensors 20 (for example, distributed throughout a patient's body and/or on or within the phrenic nerve stimulation leads 14, and/or the airway nerve stimulation leads 16).

Electrical stimulation may be delivered to a target nerve through devices such as transvenous lead electrodes and nerve cuffs. A transvenous lead is an electrical lead that is implanted within a lumen, such as a vein, near a target nerve. The transvenous lead includes electrodes and serves to position those electrodes in the vein to deliver electrical stimulation across the wall of the vein (that is, transvenously) to the target nerve.

Alternatively or additionally, a nerve cuff may be implanted around a target nerve. A nerve cuff includes electrodes and is surgically implanted so that the electrodes are in direct physical contact with the target nerve when the nerve cuff is positioned around the target nerve. The nerve cuff may also be coupled to an electrical lead. Both the electrodes on the transvenous leads and the electrodes on the nerve cuff receive an electrical stimulation signal from a pulse generator. The pulse generator may be implanted and physically coupled to the electrodes via the electrical leads. For example, the transvenous lead may couple to an implanted pulse generator and/or the nerve cuff may couple to the implanted pulse generator via an electrical lead. Alternatively or additionally, the pulse generator may be external and may wirelessly provide the generated stimulation pulse to the electrodes (for example, via one or more antennas, a magnetic field, and so forth) on the transvenous lead and/or the nerve cuff.

Electrical stimulation of various nerves may also treat conditions other than disordered breathing due to sleep apneas. The exemplary devices described herein are not limited to treatment of disordered breathing due to sleep apneas as other implementations are contemplated. For example, these other implementations may include nerve stimulation to treat loss of diaphragm control due to paralysis or a neurodegenerative disease (for example, stimulation of the phrenic nerve), and nerve stimulation to wean patients from a ventilator (for example, stimulation of the phrenic nerve), to name a few examples not limiting of the disclosure

In various examples, the implantable device 12 generates a stimulation signal (for example, a pulse train) and delivers the electrical energy to one or more implanted leads (for example, the leads 13) to electrically stimulate the one or more nerves in the patient's body. In various examples, the treatment system 10 includes one or more controllers and/or processors, such as the processor 30, configured to process information and control operation of the treatment system 10, such as by delivering and modifying the treatment.

Where the electrical stimulation is to be applied may depend on a condition that the treatment is intended to address. For example, if the treatment is to address decreased airway patency, then electrical stimulation may be applied to a hypoglossal nerve, an ansa cervicalis nerve, a C12 group nerve, a C10 group nerve, a combination thereof, and/or another nerve or group of nerves that modulate airway tone. If the treatment is to address decreased respiratory drive, then electrical stimulation may be applied to a phrenic nerve of the patient. In other examples, electrical stimulation may be applied to additional and/or different nerves or other tissues to address decreased respiratory drive and/or decreased airway patency.

In an implementation, the phrenic nerve stimulation leads 14 may be configured to stimulate nerves related to diaphragm control for treatment of decreased respiratory drive. The airway nerve stimulation leads 16 may be configured to stimulate nerves related to airway tone for treatment of decreased airway patency. For example, the phrenic nerve stimulation leads 14 may be proximate to and/or operatively coupled to one or more phrenic nerves of the patient such that electrical energy provided by the implantable device 12 conveys a stimulation, via the phrenic nerve stimulation leads 14, to the one or more phrenic nerves.

The airway nerve stimulation leads 16 may be proximate to and/or operatively coupled to one or more hypoglossal nerves of the patient such that electrical energy provided by the implantable device 12 conveys a stimulation, via the airway nerve stimulation leads 16, to the one or more hypoglossal nerves. In some examples, the airway nerve stimulation leads 16 may be proximate to and/or operatively coupled to one or more ansa cervicalis nerves in addition to, or in lieu of, the hypoglossal nerves of the patient such that electrical energy provided by the implantable device 12 conveys a stimulation, via the airway nerve stimulation leads 16, to the one or more ansa cervicalis nerves. In some examples, the airway nerve stimulation leads 16 may be proximate to and/or operatively couple to a C12 group nerve, a C10 group nerve, a combination thereof, and/or another nerve or group of nerves that modulate airway tone.

Electrical stimulation of the hypoglossal nerve and/or the ansa cervicalis nerve, which stimulate tongue and soft palate movement respectively, provide therapy for reduced airway patency due to obstruction by the tongue and/or soft palate. Stimulating the aforementioned nerves may cause the tongue and/or soft palate to move, clearing or stiffening the patient's airway. For example, stimulating a hypoglossal nerve may modify the position and/or shape of the tongue, such as by causing the tongue to protrude, retract, curve, widen, or narrow. An electrical stimulation may be applied to the hypoglossal nerve to activate cause a change the position and/or shape of the tongue to avoid a situation in which the tongue is obstructing a patient's airway.

In various examples, the leads 14 and 16 may be thin conductors, such as wires, coupled to and/or including one or more electrodes. A lead including an electrode may be implanted in the patient via a physiological lumen (that is, a cavity within a tubular organ or part) to a position proximate to a target nerve. For example, the lumen may be a blood vessel, a vein, an artery, a lymphatic vessel, and so forth.

A lead may include one electrode or multiple electrodes. A position of the electrode(s) may be selected to correspond to a position of a target tissue relative to the lead when the lead is implanted such that an electrical energy provided to the electrode delivers a stimulation to the target tissue. Target tissue may include one or more nerves and/or one or more muscles depending on the implementation. The electrodes in this configuration deliver a trans-lumen stimulation. In other words, the stimulation is delivered through the wall of the physiological lumen.

For example, a lead including an electrode may be implanted within a blood vessel of the patient and electrical energy provided to the electrode may result in electrical stimulation being applied to the target tissue transvenously. As such, this electrode may be referred to as a transvenous electrode in contrast to a nerve cuff which includes electrodes delivering a direct stimulation to the nerve without passing through a vessel wall.

In some examples, a lead includes at least one terminal pin that connects to the IPG 12, a conductor that extends along the length of the lead (that is, the lead body) from at least one terminal pin to one or more electrodes, and the one or more electrodes. The one or more electrodes are disposed along the lead and may be at the distal end of the lead. The lead may further include a central lumen (for example, a lead lumen or a bore within the physical structure of the lead and/or lead system).

In operation, an energy pulse train generated by the IPG 12 travels along the lead via the conductor to the one or more electrodes. The one or more electrodes may be in contact with the target tissue (for example, in contact with a nerve via a nerve cuff) and/or located proximately to the target tissue (for example, proximate to a nerve due to a position in a vein that is proximate to the nerve). When in contact with the target tissue, the energy pulse train stimulates the target tissue via direct contact. When proximate to the target tissue due to a position in a blood vessel, the energy pulse train stimulates the target tissue transvenously.

In some examples, multiple electrodes may be positioned close together in a location corresponding to a position of the target tissue. For example, when viewing a radial cross-section of a spiral or coiled portion of a lead, all of the electrodes of the lead may be located within a single quadrant of the radial cross-section. Transvenous electrodes may include at least one of various electrodes, such as ring electrodes (for example, electrodes spanning around at least a portion of the circumference of the lead), tip electrodes (for example, electrode spanning at least a portion of a distal cross-sectional area of the lead), paddle electrodes, electrode arrays, and/or other types of electrodes.

In various examples, one or more of the leads 13 may include one or more nerve cuffs in lieu of transvenous electrodes. A lead may include at least one terminal pin that connects to the IPG 12, a conductor that extends along the length of the lead (that is, the lead body) from at least one terminal pin to one or more nerve cuffs, and the one or more nerve cuffs.

A nerve cuff may be implemented around a target nerve or nerves. The nerve cuff includes one or more electrodes positioned in a cuff structure. Once implanted, the nerve cuff may be disposed circumferentially around at least a portion of a target nerve. The nerve cuff may be considered to be operatively coupled to the target nerve. In some examples, being operatively coupled may describe a relationship between a nerve cuff and a target nerve in which electrical stimulation provided by the nerve cuff stimulates the target nerve, optionally by at least a threshold amount of stimulation. The threshold amount of stimulation may correspond to a level at which an electrical stimulation output by the nerve cuff modulates one or more muscles innervated by the nerve. For example, a nerve cuff disposed around the phrenic nerve may be operatively coupled to the phrenic nerve where an electrical stimulation provided by the nerve cuff to the phrenic nerve causes the diaphragm to contract.

The leads 13 may be implanted in accordance with various configurations, such as by including only transvenous electrodes, only nerve cuffs, and/or combinations thereof. Where there are multiple leads 13, the various leads may provide transvenous electrodes on some leads and nerve cuffs on other leads. With transvenous electrodes, leads 13 may be disposed within various blood vessels in a patient. For example, these vessels may include one or more of the brachiocephalic vein, the superior vena cava, the internal jugular vein, the subclavian vein, the lingual vein, the ranine vein, the pericardiophrenic vein, a combination of the foregoing, and/or other blood vessels. Where any of the listed veins include a right and a left occurrence, the vessels exemplified include both the right and left occurrences. For example, the aforementioned reference to the internal jugular vein may refer to either or both of the right and left internal jugular vein, and the one or more of the leads 13 may be disposed within either or both of the right and left internal jugular vein.

With nerve cuff leads, the leads may be routed through the internal structure of a patient so as to connect the implantable pulse generator 12 with the target nerve. During implantation, an implanter (for example, a medical practitioner) may expose a nerve, preferably a nerve close to the exterior surface of a patient's body, and route the lead with the nerve cuff to this exposed nerve.

Accordingly, the IPG 12 may generate an electrical stimulation energy (for example, based on control signals provided by the processor 30) and deliver the electrical stimulation energy to the leads 13 to provide electrical stimulation to the target nerve. In some examples, the electrical stimulation circuitry 24 may generate at least one energy pulse train to be delivered to the leads 13. The stimulation circuitry 24 may draw power from the power source 27 to generate at least one energy pulse train. The processor 30 may control the stimulation circuitry 24 to generate at least one energy pulse train with one or more desired parameters. Such parameters may include, for example, a pulse amplitude, a number of pulses in a pulse train, a pulse width of the pulses, a time between pulse trains, a waveform of each pulse, a maximum pulse amplitude, a pulse period (or frequency), a stimulation current, a stimulation voltage, and a stimulation polarity (for example, monophasic or biphasic), stimulation energy ramps, and so forth.

In various examples, the implantable system 10 may provide stimulation therapy in an asynchronous mode or a synchronous mode. The implantable system 10 may be configured to select one of the asynchronous mode or the synchronous mode to operate in at a given time. In either mode, the IPG 12 may modulate or titrate the stimulation energy according to the various examples provided herein. The asynchronous mode is a mode of therapy delivery in which the stimulation rate is pre-determined and stimulation is not triggered by sensed respiration. Therefore, the stimulation is considered to be asynchronous with the patient's intrinsic breathing rate but, rather, synchronized with the stimulation rate. The synchronous mode is a respiration triggered mode where the stimulations are triggered by sensed respiration and thus stimulation may be synchronized to a patient's intrinsic breathing rate.

In the asynchronous mode, the stimulation rate (as determined by the pulse train period 1466) of the electrical stimulation applied to the phrenic nerve (and/or other nerves, such as the hypoglossal and/or ansa cervicalis nerve(s)) may be programmed to a predetermined value and/or vary within a predetermined range such that the trigger for delivery of each pulse train envelope is the programmed time interval. For example, predetermined values and/or ranges may be set by a physician based on information acquired during a monitoring period. Importantly, this pulse-train-delivery trigger excludes a sensed inspiration or other sensed respiratory parameter by the patient. In other words, the implanted system may not deliver the stimulation as a response triggered by a respiratory parameter detected by a respiration sensor such as a flow sensor, a transthoracic impedance sensor, a blood oxygen sensor, and/or other physiological sensor. In the asynchronous mode, the implantable system 10 may provide the phrenic nerve stimulation at an entrainment frequency.

FIG. 13 illustrates an entrainment index calculation according to an example. This figure illustrates a histogram 40 of a number of breath periods 42 versus breath periods 44. A range of breath periods may be predetermined as a synchronized range 46 that includes the stimulation rate 48. Breaths inside this range may be designated as synchronized and breaths outside of this range may be designated as unsynchronized. The total number of breaths within the synchronized range 46 provides the number of target breath periods. The entrainment index may be calculated as a fraction or percentage using the number of target breath periods divided by a total number of breaths. Thus, in the example shown in FIG. 1B, the total number of breaths is 25, the number of breaths inside the synchronized range is 15, and the number of breaths in the unsynchronized range is 10. Therefore, the entrainment index for this example is 0.6 or 60%. A target entrainment index may be between 0.6 and 0.8 A (or 60%-80%). The value of the entrainment index is expected to increase proportionally with the entrainment of respiration by stimulation. Thus, a higher entrainment index indicates a higher degree of entrainment by stimulation.

A degree of entrainment for a patient may be indicated by an entrainment index. The entrainment index may quantify or otherwise provide a metric characterizing a patient's response to entrainment therapy. This entrainment index may indicate the extent to which the entrainment therapy has entrained the patient's respiration and synchronized the patient's respiration to an entrainment stimulation frequency. The entrainment index may be an index of a degree to which the patient's respiration rate is captured within a target range for breathing rate as determined by the entrainment therapy (for example, a capture index).

An example of an entrainment index, not limiting of the disclosure, is a quantitative metric of how well a patient's breathing is synchronized with a stimulation pulse frequency. In an implementation, the entrainment index may be determined based on breathing-rate parameters. For example, a transthoracic-impedance sensor of at least one of the sensors 18, 19, and/or 20 may acquire a breathing rate.

As one example, the entrainment index may be calculated by dividing the spectral power in the stimulation frequency band (SFB) by the spectral power in the intrinsic respiratory frequency band (RFB). For example, the RDS diagnostics engine 26 of the external computing device 210 may determine an entrainment index based on spectral power analysis. The entrainment index represents the fraction of a total spectral plot of respiration that falls into a frequency band proximate and/or including the stimulation frequency. The value of the entrainment index is expected to increase proportionally with the entrainment of respiration by stimulation. Thus, a higher entrainment index indicates a higher degree of entrainment by stimulation. The stimulation frequency thus becomes the dominant frequency of the respiration signal as entrainment increases. When stimulation entrains respiration, the spectral power of the respiratory signal in the immediate range of the driving frequency will be higher than in other frequency bands. In an implementation, a target entrainment index may be 0.6-0.8 A (or 60%-80%).

As another example, the entrainment index may be calculated based on a quantity of measured breaths occurring at a rate that is close to the stimulation. For example, the RDS engine 34 of the implantable treatment system 10 may determine an entrainment index based on measured breaths. Calculation of spectral power may be computationally heavy and therefore may not be well-suited for an implanted device powered by an internal power source (for example, a battery) where power conservation is an important factor. The measured breaths procedure may be less computationally heavy and therefore preserve battery life for the power source 27.

As an example of a measured breath calculation, the implantable treatment system 10 may measure peak-to-peak timings in the respiratory signal to determine a length of each breath. The system 10 may collect data for a series of breath lengths over a set amount of time (for example, one to five minutes) and classify each breath as entrained or unentrained depending on whether the breath length is within a predetermined tolerance range of the stimulation rate. At the end of the collection time, the system 10 may calculate a number of entrained breaths divided by a total number of breaths. The system 10 may compare this ratio to a pre-determined threshold defining successful synchronization, or entrainment. For example, the threshold may be between 0.6-0.8 A. If the ratio meets or exceeds the threshold or falls within a target range, the system 10 may determine the patient to be entrained.

FIG. 14 illustrates an example of an electrical stimulation trace 1490. The electrical stimulation includes a series of stimulation pulses 1480. Each stimulation pulse 1480 may be provided as a pulse train 1491 where the stimulation pulse 1480 is a pulse envelope for the individual pulses (for example, a number n of individual pulses 1485a, 1485b, . . . , 1485n) of the pulse train 1491. Each stimulation pulse 1480 may be referred to as a stimulation burst 1480 as the pulse train 1491 provides a burst of individual pulses. The leads 13 may each include electrodes to deliver electrical stimulation to a target nerve according to the electrical stimulation profile that specifies the various parameters or characteristics illustrated for the electrical stimulation trace 1490. For example, the processor 50 may control the stimulation circuitry 24 to provide the electrical stimulation pulses to the leads 13. The stimulation pulses 1480 are delivered at a stimulation pulse period corresponding to a stimulation rate. The stimulation rate (for example, the rate of the stimulation pulses 1480 or stimulation bursts 1480) may be about 0.1-0.5 Hz in correspondence with a desired stimulated breathing rate of 10-18 breaths per minute. In some examples, a stimulation rate indicated by a stimulation profile may be determined based on a predetermined unstimulated respiration rate, that is, a predetermined respiration rate when stimulation is not applied.

For example, the stimulation rate may be equal to the predetermined unstimulated respiration rate, or may be greater or less than the predetermined unstimulated respiration rate. In one example, the stimulation rate may be a predetermined number of cycles-per-minute fewer than the predetermined unstimulated respiration rate (for example, one cycle-per-minute fewer, two cycles-per-minute fewer, three cycles-per-minute fewer, or some other value). In other examples, the stimulation rate is a predetermined number of cycles-per-minute greater than the predetermined unstimulated respiration rate (for example, one cycle-per-minute greater, two cycles-per-minute greater, three cycles-per-minute greater, or some other value). A stimulation pulse 1480 may be 1-4 seconds in total duration. In the example of FIG. 14, the total duration of the stimulation pulse 1480 is the pulse train duration 1450. For example, the stimulation pulse may be about 2 seconds in total duration. Each stimulation pulse 1480 may be provided as a pulse train of individual pulses, each individual pulse having a pulse train period 1466 (also referred to as an individual pulse train period 1466) corresponding to a pulse train frequency (also referred to as an individual pulse frequency). The pulse train frequency (that is, the frequency of the individual pulses within the stimulation pulse 1480 or stimulation burst 1480) may be 10-40 Hz.

A selected electrical stimulation profile may specify parameters including one or more of the stimulation rate (corresponding to the stimulation pulse period 1465), the pulse train duration 1450, and the pulse train frequency (corresponding to the pulse train period 1466). Additionally, the parameters may include a time between stimulation pulses 1430. The pulse train envelope forming each stimulation pulse 1480 defines the maximum pulse amplitude 1440 as the maximum amplitude of one or more individual pulses within the pulse train; accordingly, the maximum pulse amplitude 1440 may also be referred to as a maximum individual pulse amplitude. Each individual pulse may be further characterized by a pulse width 1460, also referred to as an individual pulse width 1460.

As illustrated in FIG. 14, the individual pulses of the pulse train may not all have the same pulse amplitude 1445 (also referred to as an individual pulse amplitude 1445) and may vary in amplitude so as to ramp up 1498 the energy of the stimulation pulse 1480 and/or ramp down 1499 the energy of the stimulation pulse 1480. The ramp up may occur over a ramping-up time, for example, the time interval for the ramping up pulses 1498. The ramp down may occur over a ramping-down time, for example, the time interval for the ramping down pulses 1499. In some examples, the stimulation pulse 1480 may not include a ramp up and/or a ramp down. The IPG 12 configuration with regard to operational parameters and/or with regard to stimulation circuitry 24 may exclude a square wave capability for an electrical stimulation profile in which the pulse trains exclude both the ramping-up period and the ramping-down period.

The electrical stimulation delivered by the IPG 12 may be characterized by one or more electrical stimulation parameters as specified by the electrical stimulation profile. The IPG 12 may adjust one or more of these parameters to titrate the electrical stimulation energy delivered to a target nerve. The electrical stimulation parameters may include pulse train parameters. The pulse train parameters, as discussed above, may include one or more of a pulse amplitude 1445 (also referred to as an individual pulse amplitude 1145), a maximum pulse amplitude 1440 (also referred to as a maximum individual pulse amplitude 1440), a pulse train period (or frequency) 1466 (also referred to as an individual pulse train period or individual pulse frequency 1466), a pulse width 1460 (also referred to as an individual pulse width 1460), the number n of individual pulses 1485a, 1485b, . . . 1485n in each pulse train envelope forming each stimulation pulse 1480, and/or a pulse train duration 1450. The pulse amplitude 1445 may include an amplitude of at least one of the ramping-up pulses, an amplitude of the constant-amplitude pulses, and/or an amplitude of at least one of the ramping-down pulses and may thus include multiple different pulse amplitudes. The pulse train parameters may further include one or more of a time over which an amplitude of the pulses is ramped up, a time over which the amplitude of the pulses is ramped down, a number of ramping-up pulses, and/or a number of ramping-down pulses.

The stimulation parameters may further include one or more of the stimulation rate corresponding to the stimulation pulse period 1465, the time between stimulation pulses 1430, a stimulation current, a stimulation voltage, and a stimulation polarity (for example, monophasic or biphasic), stimulation energy ramps, and so forth. The stimulation energy ramps may include changes in energy delivered by a stimulation pulse 1480 over a series of stimulation pulses 1480. One or more electrical stimulation parameters may be adjusted to change the energy delivered by stimulation pulses 1480 over time to titrate the stimulation energy delivered to the target nerve. Such titration may achieve a desirable balance between patient comfort and stimulation efficacy. For example, more intense electrical stimulation of a patient's phrenic nerve may yield higher activation of a patient's diaphragm, which may increase the efficacy of nerve stimulation therapy in some examples. The stimulation profile may also include titration parameters for titration of stimulation energy over a period of minutes, hours, days, months, and so forth.

However, more intense electrical stimulation may also yield a greater sensory response from a patient. For example, a patient may feel more discomfort as the stimulation intensity is increased. Experiencing discomfort may adversely impact a patient's sleep. For example, even if a particular set of stimulation parameters effectively activate the patient's diaphragm, treatment according to that particular set of stimulation parameters may be contraindicated if the particular set of stimulation parameters causes the patient to wake up and lose sleeping time, for example due to discomfort produced by the particular set of stimulation parameters. It is therefore beneficial to the efficacy of stimulation therapy to identify stimulation parameters that produce a desired muscle response (for example, diaphragm contraction and expansion) while minimizing or avoiding patient discomfort.

In an implementation, the IPG 12 may ramp up the stimulation amplitude to increase the intensity of the stimulation, or may ramp down the stimulation amplitude to decrease the intensity of the stimulation. In general, the stimulation amplitude may vary over a range from a minimum amplitude for a pulse train to a maximum amplitude for the pulse train. The individual pulses within the pulse train may vary in amplitude but the maximum amplitude defines the maximum amplitude reached during a stimulation pulse train. In at least one example, individual pulse train pulses that constitute a stimulation pulse may ramp up for a period of time and may ramp down for a period of time. In various examples, a start time of a pulse train may refer to a time at which a pulse train begins to ramp up, and an end time of a pulse train may refer to a time at which a pulse train finishes ramping down.

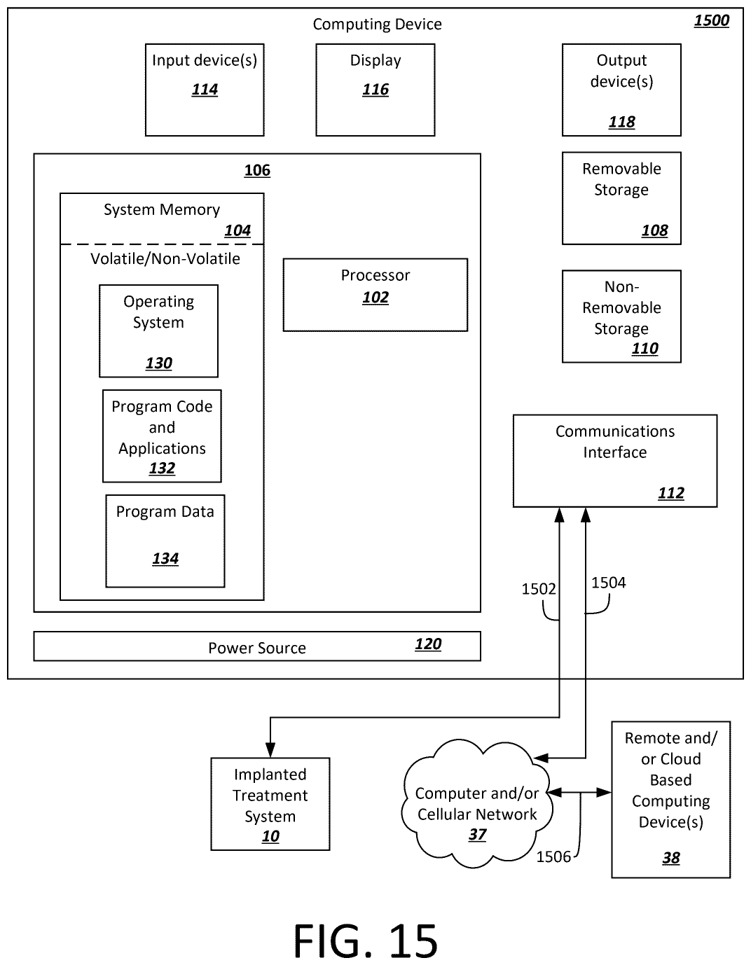

FIG. 15 illustrates a block diagram of the computing device 1500 according to an example. The computing device 1500 may illustrate an example of the computing device 210 and/or the computing device 216. While in some examples the computing device 1500 is a tablet type of computer or a mobile phone such as a smartphone, the computing device 1500 can include other types of computers and is therefore described in the context of a general computing device. In its most basic configuration, the computing device 1500 includes at least a processing unit 102 and a memory 104. Depending on the exact configuration and type of computing device, the memory 104 may be volatile (such as RAM), non-volatile (such as ROM, flash memory, and so forth) or some combination of the two. This most basic configuration is illustrated in FIG. 15 within box 106.

Additionally, the computing device 1500 may also have additional features and/or functionality. For example, the computing device 1500 may also include additional storage (removable and/or non-removable) including, but not limited to, magnetic or optical disks or tapes, USB flash drives, memory cards, and so forth. Such additional storage is illustrated in FIG. 15 by a removable storage 108 and a non-removable storage 110. Computer-storage media may include volatile and/or nonvolatile media, removable and/or non-removable media, and so forth, implemented in any method or technology for storage of information such as computer-readable instructions, data structures, program modules or other data. The memory 104, the removable storage 108, and the non-removable storage 110 are all examples of computer-storage media, also referred to as non-transitory computer-readable media. Computer-storage media includes, but is not limited to, RAM, ROM, EEPROM, flash memory or other memory technology, CDROM, digital versatile disks (DVD) or other optical storage, magnetic cassettes, magnetic tape, magnetic disk storage or other magnetic storage devices, or any other medium that can be used to store the desired information and which can be accessed by the computing device 1500. Any such computer-storage media may be part of computing device 1500.

System memory 104 may include operating system 130, one or more programming modules or applications 132, and program data 134. Operating system 130, for example, may be suitable for controlling the operation of the computing device 1500. As stated above, a number of program modules 132 and program data files 134 may be stored in system memory 104, including operating system 130. While executing on processing unit 102, programming modules or applications 132 may perform processes including, for example, one or more methods described herein, using one or more of the GUI screens or windows shown and described herein. For example, where the computing device 1500 is an example of the external computing device 210, the processing unit 102 may include and/or execute the RDS diagnostics engine 26.

Program modules or applications may include routines, programs, components, data structures, and other types of structures that may perform particular tasks or that may implement particular abstract data types. Moreover, disclosed examples may be practiced with other computer-system configurations, including multiprocessor systems, microprocessor-based or programmable consumer electronics, minicomputers, mainframe computers, and the like. Disclosed examples may also be practiced in distributed computing environments where tasks are performed by remote processing devices linked through a communications network. In a distributed computing environment, program modules may be located in both local and remote memory storage devices.

Furthermore, some disclosed examples may be practiced in an electrical circuit including discrete electronic elements, packaged or integrated electronic chips containing logic gates, a circuit utilizing a microprocessor, or on a single chip containing electronic elements or microprocessors. Some disclosed examples may also be practiced using other technologies capable of performing logical operations such as, for example, AND, OR, and NOT, including but not limited to mechanical, optical, fluidic, and quantum technologies.

The computing device 1500 may also contain a communications interface 112 that allow the device to communicate with other devices. The communications interface 112 can include, for example, wired media connections such as a wired network or direct-wired connection, and wireless media connections such as acoustic, RF, infrared, and other wireless media connections. In some examples, communications interface 112 is configured to provide communication (for example, wireless communication) via a connection 1502 between the computing device 1500 and the IPG 12 of treatment system 10. For example, such communications may include processor instructions provided by a programmer of the IPG 12 to select particular anode-cathode pairs corresponding to a desired current vector (that is, to implement current steering). Thus, in some examples, the communication interface 112 may include circuitry configured to provide communication connection as a wireless communication connection as described above.

In still other examples, such as in which the computing device 1500 is coupled with or replaced by remote or cloud-based computing device(s) 38 for some or all of the processing functions described herein, communication interface 112 can communicate via a connection 1504 to an internet connection or other network 37, to the computing device(s) 38, which is communicatively coupled to the network 37 via a connection 1506. In still other examples, the processing functions described herein are performed without the use of a local computing device 1500, and are instead incorporated into the treatment system 10 and/or the computing device(s) 38. In some examples, the treatment system 10 performs at least a portion of the processing functions described herein, and a remaining portion, if any, of the processing functions may be performed by the computing device 1500 and/or one or more alternative or additional computing devices, such as the remote or cloud-based computing devices 38. The description herein is provided in the context of processing functions being provided at least partially by computing device 1500, but those of skill in the art will understand that such functions can be implemented outside of computing device 1500.

In some examples the computing device 1500 has, or can be coupled to, a touch screen display device 116 which provides a touch-based GUI. The computing device 1500 may also have, or be coupled to, one or more input devices 114, such as a keyboard, mouse, pen, voice input device, and so forth, for providing other input (for example, user feedback) to the computing device 1500. The computing device 1500 may be coupled to one or more other output devices 118 such as speakers, a printer, a vibration generator, and so forth. Further, display device 116, input devices 114 and output devices 118 can all be considered to be separate from, or alternatively part of, the computing device 1500. The computing device 1500 can be provided with a portable or non-portable power source 120, such as a battery pack, a transformer, a power supply, or the like. The power source 120 provides power for computations, communications and so forth by the computing device 1500.

Having thus described several aspects of at least one embodiment, it is to be appreciated various alterations, modifications, and improvements will readily occur to those skilled in the art. Such alterations, modifications, and improvements are intended to be part of, and within the spirit and scope of, this disclosure. Accordingly, the foregoing description and drawings are by way of example only.

1.-170. (canceled)

171. A system for providing sleep disordered breathing treatment for a patient, the system comprising:
one or more stimulation leads, each stimulation lead being implantable in the patient and configured to receive an electrical stimulation energy, wherein at least one stimulation lead is configured to deliver electrical stimulation to at least one phrenic nerve based on the received electrical stimulation energy; and
at least one implantable electrical pulse generator configured to be coupled to the one or more stimulation leads and comprising:
wireless communication circuitry,
at least one power source configured to generate the electrical stimulation energy,
stimulation circuitry coupled to the at least one power source and configured to provide the electrical stimulation energy to the one or more stimulation leads,
at least one sensor configured to generate at least one signal indicative of a degree of arousal of the patient,
memory, and
at least one processor coupled to the wireless communication circuitry, the at least one power source, the stimulation circuitry, the at least one sensor, and the memory, the at least one processor being configured to:
receive the at least one signal indicative of the degree of arousal of the patient,
process the at least one signal to detect a change in the degree of arousal, and
in response to the detected change in the degree of arousal, cause the stimulation circuitry to titrate the electrical stimulation energy, wherein the titration comprises one or more adjustments of the electrical stimulation energy between a minimum effective energy and a target maximum treatment energy.

172. The system of claim 171, wherein the at least one sensor comprises at least one accelerometer and wherein the at least one signal indicative of the change in the degree of arousal comprises an acceleration signal indicative of patient movement information.

173. (canceled)

174. (canceled)

175. The system of claim 172, wherein the patient movement information comprises rolling information comprising at least one of a number of rolls, a frequency of rolls, a magnitude of rolls, or a cumulative number of rolls in a respective time period.

176. (canceled)

177. The system of claim 17_5, wherein:
a first degree of arousal corresponds to the at least one of the number of rolls, the frequency of rolls, the magnitude of rolls, or the cumulative number of rolls in a first respective time period subceeding a respective threshold, and
a second degree of arousal corresponds to the at least one of the number of rolls, the frequency of rolls, the magnitude of rolls, or the cumulative number of rolls in a second respective time period exceeding the respective threshold.

178. The system of claim 175, wherein the magnitude of rolls is indicative of movement of the patient relative to positional quadrants, and wherein the positional quadrants comprise a prone position, a supine position, a right-side position, and a left-side position.

179. (canceled)

180. The system of claim 178, wherein:
a first degree of arousal corresponds to patient motion within a first positional quadrant;
a second degree of arousal corresponds to patient motion between the positional quadrants; and
a third degree of arousal corresponds to patient motion above a threshold amount irrespective of positional quadrant.

181. The system of claim 175, wherein the at least one processor is configured to detect a deviation of the rolling information from a baseline movement profile for the patient while sleeping and titrate the electrical stimulation energy based on the deviation of the rolling information.

182. The system of claim 181, wherein the detection of the deviation of the rolling information triggers the titration of the electrical stimulation energy.

183. (canceled)

184. The system of claim 181, wherein the deviation from the baseline movement profile comprises one or more of:
a) a present number of rolls over a present rolling window of time exceeding or subceeding a baseline number of rolls indicated by the baseline movement profile over a baseline rolling window of time by at least a threshold number of rolls, wherein the present rolling window of time is equal to the baseline rolling window of time;
b) a present frequency of rolls exceeding or subceeding a baseline frequency of rolls indicated by the baseline movement profile by at least a threshold frequency of rolls;
c) a present magnitude of one or more rolls exceeding or subceeding a baseline magnitude of one or more rolls indicated by the baseline movement profile by at least a threshold magnitude, wherein the present magnitude of the one or more rolls and the baseline magnitude of one or more rolls are each measured by a number of angular degrees rolled in a respective roll; or
d) a present cumulative number of rolls over a present reference period of time exceeding or subceeding a baseline cumulative number of rolls indicated by the baseline movement profile over a baseline reference period of time by at least a threshold cumulative number of rolls,
wherein the present reference period of time and the baseline reference period of time each comprise a sleeping time period between the patient falling asleep and waking up.

185. (canceled)

186. (canceled)

187. The system of claim 184, wherein the at least one processor is configured to adjust over time one or more of the threshold number of rolls, the threshold frequency of rolls, the threshold magnitude, or the threshold cumulative number of rolls.

188. The system of claim 172 wherein the patient movement information comprises translation information.

189. The system of claim 171, wherein the one or more adjustments comprise a decrease in the electrical stimulation energy from a first energy to a second energy in response to an increase in the degree of arousal, the second energy being non-zero and being equal to or greater than the minimum effective energy.

190. The system of claim 171, wherein the one or more adjustments comprise an increase in the electrical stimulation energy from a first energy to a second energy over a plurality of steps, the first energy being equal to or greater than the minimum effective energy and the second energy being equal to or less than the target maximum treatment energy, wherein each step of the plurality of steps comprises a change in energy.

191. The system of claim 171, wherein the one or more adjustments comprise a change in the electrical stimulation energy from a first energy to a second energy over a plurality of steps, and wherein the one or more adjustments comprise at least one of (a) an adjustment of a quantity of steps included in the plurality of steps or (b) a magnitude of at least one step of the plurality of steps.

192. The system of claim 191, wherein the one or more adjustments comprise
(a) an incremental decrease in a magnitude of the at least one step of the plurality of steps as the electrical stimulation energy approaches the target maximum treatment energy, and
(b) an incremental increase in a quantity of steps included in the plurality of steps as the electrical stimulation energy approaches the target maximum treatment energy.

193. The system of claim 171, wherein the at least one processor is further configured to:
process the at least one signal to detect an absence of change in the degree of arousal, and
in response to the detected absence of change in the degree of arousal, cause the stimulation circuitry to increase the electrical stimulation energy between the minimum effective energy and the target maximum treatment energy.

194. The system of claim 171, wherein the titration of the electrical stimulation energy comprises an adjustment of at least one hold time duration at a particular electrical stimulation energy.

195. The system of claim 171, wherein the minimum effective energy comprises a minimum energy that, when applied to the at least one phrenic nerve, modulates the diaphragm of the patient, and wherein the minimum effective energy comprises a plurality of minimum effective energies, and wherein each minimum effective energy of the plurality of minimum effective energies is associated with a respective one of a prone position of the patient, a supine position of the patient, a right-side position of the patient, and a left-side position of the patient.

196. (canceled)

197. (canceled)

198. The system of claim 171, wherein the at least one sensor comprises at least one transthoracic impedance sensor configured to generate at least one feedback signal indicative of respiration of the patient, and wherein the at least one processor is coupled to the at least one sensor, and wherein the titration comprises at least one adjustment of the electrical stimulation energy based on the at least one feedback signal.

199. (canceled)

200. (canceled)

201. The system of claim 198, wherein the at least one processor is further configured to:
determine an entrainment index based on the at least one feedback signal; and
compare the entrainment index to a threshold value,
wherein the one or more adjustments comprise a decrease in the electrical stimulation energy responsive to the entrainment index exceeding the threshold value.

202. (canceled)

203. (canceled)

204. The system of claim 198,
determine an entrainment index based on the at least one feedback signal; and
compare the entrainment index to a threshold value,
wherein the one or more adjustments comprise an increase in the electrical stimulation energy responsive to the entrainment index subceeding the threshold value.

205. The system of claim 198,
determine an entrainment index based on the at least one feedback signal; and
compare the entrainment index to a threshold value,
wherein the titration comprises maintaining a magnitude of the electrical stimulation energy responsive to the entrainment index being within a range of values around the threshold value.

206. The system of claim 205, wherein the target maximum treatment energy is an energy at which the entrainment index is within the range of values around the threshold value.

207. The system of claim 171, wherein the stimulation circuitry is configured to provide the electrical stimulation energy to the one or more stimulation leads in an asynchronous mode.

208. The system of claim 171, wherein the at least one stimulation lead is at least one first lead and wherein the system further comprises at least one second lead configured to deliver electrical stimulation to an upper airway nerve.

209. The system of claim 208, wherein the upper airway nerve comprises a hypoglossal nerve.

210. The system of claim 208, wherein the at least one implantable electrical pulse generator is configured to support dual channel operations, wherein a first channel is configured to control stimulation via the at least one first lead and a second channel is configured to control stimulation via the at least one second lead.

211. The system of claim 210, wherein the at least one processor is configured to cause the stimulation circuitry to titrate the electrical stimulation energy for each channel of the first channel and the second channel based at least in part on a target nerve, wherein the target nerve for the first channel is the at least one phrenic nerve and the target nerve for the second channel is the upper airway nerve.

212. The system of claim 171, wherein the titration of the electrical stimulation energy comprises an adjustment of at least one of the minimum effective energy or the target maximum treatment energy over time.

213. The system of claim 171, wherein the at least one processor is configured to cause the stimulation circuitry to titrate the electrical stimulation energy based on a predicted patient response to the change in the degree of arousal.
